(19) United States
(12) Patent Application Publication
(10) Pub. No.: US 2024/0374310 A1
(43) Pub. Date: Nov. 14, 2024

(54) SYSTEMS, APPARATUS AND METHODS FOR FORMING INCISIONS IN TISSUE USING LASER PULSES

(71) Applicant: LIGHT MATTER INTERACTION INC., ETOBICOKE (CA)

(72) Inventors: DARREN KRAEMER, TORONTO (CA); R.J. DWAYNE MILLER, PORT CREDIT (CA); HOLLY XU, TORONTO (CA)

(21) Appl. No.: 18/686,525

(22) PCT Filed: Aug. 26, 2022
(86) PCT No.: PCT/CA2022/051294

§ 371 Date: Feb. 26, 2024

Related U.S. Application Data
(60) Provisional application No. 63/237,740, filed on Aug. 27, 2021.

(51) Int. Cl. A61B 18/20

(52) U.S. Cl. CPC A61B 18/203

(57) ABSTRACT

Systems, devices and methods are provided that facilitate the formation of incisions in tissue while reducing, minimizing or avoiding the generation of scar tissue. Devices are provided that facilitate the generation of an incision during multiple passes of a laser beam, such as a picosecond infrared laser (PIRL) beam. Some implementations employ the use of guidelines or guide structures to facilitate alignment of a laser beam delivery tool during the formation of an incision, optionally based on feedback provided by one or more sensors. Optical waveguide structures are disclosed for the efficient and controlled generation of laser incisions. Devices and methods are disclosed for applying tension, via manual or autonomous means, during and/or after the formation of an incision. The tension may be applied, optionally based on feedback from one or more sensors, to avoid the deformation of tissue within the incision beyond the elastic deformation limit.

### CROSS-REFERENCE TO RELATED APPLICATION

This application claims priority to U.S. Provisional Patent Application No. 63/237,740, titled “SYSTEMS, APPARATUS AND METHODS FOR FORMING INCISIONS IN TISSUE USING LASER PULSES” and filed on Aug. 27, 2021, the entire contents of which is incorporated herein by reference.

### BACKGROUND

This present disclosure relates generally to surgical methods and apparatus, and more particularly, to surgical apparatus and methods for creating incisions in skin.

Surgical incisions are essential and are often the first intervention applied to patient's physical body during surgery. The selected method of incision determines the quality of postsurgical healing, both aesthetically and functionally, as the healing outcome is largely affected by how the wound was initially introduced. Current incision techniques include energetic mechanisms such as electro-cautery and thermal laser ablation, and ‘cold’ techniques such as conventional scalpel and non-thermal laser ablation.

Regardless of the mechanism chosen, a major problem of current incision instruments such as the scalpel or electrosurgery devices is the microscopic damage caused at the edges of the incision, which ultimately lead to scar formation. Surface incisions, which form scars, are undesirable aesthetically and sometimes functionally, especially in cosmetic surgery procedures. Incisions which are internal and form scars can lead to adhesions.

### SUMMARY

Systems, devices and methods are provided that facilitate the formation of incisions in tissue while reducing, minimizing or avoiding the generation of scar tissue. Devices are provided that facilitate the generation of an incision during multiple passes of a laser beam, such as a picosecond infrared laser (PIRL) beam. Some implementations employ the use of guidelines or guide structures to facilitate alignment of a laser beam delivery tool during the formation of an incision, optionally based on feedback provided by one or more sensors. Optical waveguide structures are disclosed for the efficient and controlled generation of laser incisions. Devices and methods are disclosed for applying tension, via manual or autonomous means, during and/or after the formation of an incision. The tension may be applied, optionally based on feedback from one or more sensors, to avoid the deformation of tissue within the incision beyond the elastic deformation limit.

Accordingly, in one aspect, there is provided a system for forming an incision, the system comprising:
a laser system configured to generate and deliver laser pulses, the laser system comprising a laser pulse delivery tool having a functional distal region, the functional distal region comprising an optical waveguide for delivering the laser pulses, wherein the laser system is configured such that the laser pulses delivered by the optical waveguide are capable of ablating skin tissue when a distal tip of the optical waveguide is contacted with the skin tissue;
a sensor configured to provide signals suitable for detecting misalignment of the laser pulse delivery tool with a prescribed incision path during translation of the laser pulse delivery tool and formation of the incision; and
control and processing circuitry operably coupled to the sensor and the laser system, the control and processing circuitry comprising at least one processor and associated memory, the memory comprising instructions executable by the at least one processor for performing operations comprising:
   processing the signals in real-time to detect misalignment of the laser pulse delivery tool relative to the prescribed incision path; and
   controlling the laser system such that delivery of the laser pulses is prevented during misalignment of the laser pulse delivery tool relative to the prescribed incision path.

In some example implementations of the system, the control and processing circuitry is configured to detect a state of misalignment such that a width of the incision is maintained within two times a beam width of the laser pulses delivered onto the skin tissue when the distal tip of the optical waveguide is contacted with the skin tissue.

In some example implementations of the system, the control and processing circuitry is configured to detect a state of misalignment, during at least a first pass of the laser pulse delivery tool, when the distal tip of the optical waveguide resides beyond of a spatial extent of a preoperative incision marking defining the prescribed incision path.

The control and processing circuitry may be configured to detect the state of misalignment, during at least the first pass of the laser pulse delivery tool, when the distal tip of the optical waveguide resides beyond of a prescribed spatial offset relative to a midpoint of the preoperative incision marking defining the prescribed incision path. The prescribed spatial offset may be equal to one half of a beam width of the laser pulses delivered onto the skin tissue when the distal tip of the optical waveguide is contacted with the skin tissue.

The control and processing circuitry may be configured such that after a plurality of passes of the laser pulse delivery tool, the prescribed incision path is defined by processing the signals to determine a real-time spatial extent of the incision; defining the prescribed incision path by a central region of the incision, wherein a state of misalignment is detected when the distal tip of the optical waveguide is determined to reside beyond the central region.

In some example implementations of the system, the prescribed incision path is defined dynamically during formation of the incision.

In some example implementations of the system, the control and processing circuitry is configured such that during at least a subset of a total number of passes of the laser pulse delivery tool, the prescribed incision path is defined by processing the signals to determine a real-time spatial extent of the incision.

In some example implementations, the system further comprises an incision guide for guiding translation of the laser pulse delivery tool, wherein the incision guide is configured to be secured to the skin, the incision guide comprising an incision aperture defining the prescribed incision path, wherein the control and processing circuitry is configured detect a state of misalignment when the distal tip of the optical waveguide resides beyond of a spatial extent of the incision aperture.

In some example implementations of the system, the sensor is an imaging sensor configured to obtain a plurality of images of a skin region comprising the prescribed incision path during translation of the laser pulse delivery tool and formation of the incision, and wherein the control and processing circuitry is configured to process the plurality of images to detect misalignment of the laser pulse delivery tool relative to the prescribed incision path. The imaging sensor may be secured to the laser pulse delivery tool. The sensor may be a non-imaging sensor secured to the laser pulse delivery tool. The non-imaging sensor may be an inertial sensor. The imaging sensor may be remote from the laser pulse delivery tool, and wherein a position and orientation of the laser pulse delivery tool is trackable by a tracking system, and the signals from the imaging sensor and tracking data from the tracking system are employed to detect misalignment of the laser pulse delivery tool relative to the prescribed incision path.

In some example implementations of the system, the control and processing circuitry is configured to generate an alert when misalignment of the laser pulse delivery tool relative to the prescribed incision path is detected.

In some example implementations of the system, the control and processing circuitry is configured to control the laser pulse delivery tool to generate haptic feedback when misalignment of the laser pulse delivery tool relative to the prescribed incision path is detected.

In some example implementations of the system, the laser system is configured to generate the laser pulses with a wavelength such that absorption of a given laser pulse by the skin tissue is predominantly due to excitation of vibrational modes of one or more constituents of the skin tissue;
the laser system being configured to respectively generate and deliver the laser pulses such that when a given volume of skin tissue is irradiated by contact of the distal tip of the optical waveguide with the skin tissue:
   a pulse duration is shorter than a first time duration required for thermal diffusion out of the laser-irradiated volume of skin tissue and shorter than a second time duration required for a thermally driven expansion of the laser-irradiated volume of skin tissue;
   the pulse duration and a pulse fluence result in a peak pulse intensity below a threshold for ionization-driven ablation to occur within the laser-irradiated volume of skin tissue; and
   the pulse fluence is sufficiently high such that the laser-irradiated volume of skin tissue is ablated and such that any residual energy is insufficient to induce substantial laser-induced scar tissue formation.

In another aspect, there is provided a system for forming an incision, the system comprising:
a laser pulse delivery tool comprising:
   a moveable support; and
   an optical waveguide extending from the moveable support, the optical waveguide comprising a distal tip;
a laser source in optical communication with the laser pulse delivery tool for delivering laser pulses to the optical waveguide, such that the laser pulses are directed through the optical waveguide to the distal tip, the laser pulses being suitable for ablating skin tissue when the distal tip is contacted with the skin tissue and the laser pulses are delivered to the skin tissue through the distal tip;
the distal tip being configured such that when the distal tip contacts the skin tissue at a contact location, laser pulses emerging from the distal tip are directed at an ablation location that resides laterally adjacent to the contact location for ablating skin tissue adjacent to the contact location, the ablation location thereby residing, laterally relative to the distal tip, along an ablation direction, such that when the laser pulse delivery tool is translated in the ablation direction during formation of the incision, skin tissue residing in front of the distal tip is ablated prior to encountering the distal tip; and
alignment means for aligning the laser pulse delivery tool, relative to a skin surface of a subject, such that during formation of the incision by translation of the laser pulse delivery tool, the ablation direction is aligned with a direction of travel of the laser pulse delivery tool.

In some example implementations of the system, the alignment means comprises a guide structure that is removably attachable to the skin surface, wherein the guide structure is configured to receive and guide translation of the laser pulse delivery tool along a prescribed incision path for forming the incision, while enforcing alignment of the laser pulse delivery tool relative to the prescribed incision path such that the ablation direction is aligned with the direction of travel of the laser pulse delivery tool.

In some example implementations of the system, the alignment means comprises:
a robotic assembly configured to support the laser pulse delivery tool; and
control and processing circuitry operably coupled to the robotic assembly, the control and processing circuitry comprising at least one processor and associated memory, the memory comprising instructions executable by the at least one processor for performing operations comprising:
   controlling the robotic assembly to translate the laser pulse delivery tool relative to the skin surface for formation of the incision along a prescribed incision path while maintaining alignment of the laser pulse delivery tool relative to the prescribed incision path such that the ablation direction is aligned with the direction of travel of the laser pulse delivery tool.

In some example implementations of the system, the laser source is configured to generate the laser pulses with a wavelength such that absorption of a given laser pulse by the skin tissue is predominantly due to excitation of vibrational modes of one or more constituents of the skin tissue;
the laser source and the laser pulse delivery tool being configured to respectively generate and deliver the laser pulses such that when a given volume of skin tissue is irradiated by placement of the distal tip of the optical waveguide adjacent to the given volume of skin tissue:
   a pulse duration is shorter than a first time duration required for thermal diffusion out of the laser-irradiated volume of skin tissue and shorter than a second time duration required for a thermally driven expansion of the laser-irradiated volume of skin tissue;
   the pulse duration and a pulse fluence result in a peak pulse intensity below a threshold for ionization-driven ablation to occur within the laser-irradiated volume of skin tissue; and
   the pulse fluence is sufficiently high such that the laser-irradiated volume of skin tissue is ablated and such that any residual energy is insufficient to induce substantial laser-induced scar tissue formation.

In another aspect, there is provided a system for forming an incision, the system comprising:
a laser pulse delivery tool comprising:
   a moveable support; and
   an optical waveguide extending from the moveable support, the optical waveguide comprising a distal tip;
a laser source in optical communication with the laser pulse delivery tool for delivering laser pulses to the optical waveguide, such that the laser pulses are directed through the optical waveguide to the distal tip, the laser pulses being suitable for ablating skin tissue when the distal tip is contacted with the skin tissue and the laser pulses are delivered to the skin tissue through the distal tip;
the distal tip being configured such that when the distal tip contacts the skin tissue at a contact location, laser pulses emerging from the distal tip are directed at an ablation location that resides laterally adjacent to the contact location for ablating skin tissue adjacent to the contact location, the ablation location thereby residing, laterally relative to the distal tip, along an ablation direction, such that when the laser pulse delivery tool is translated in the ablation direction during formation of the incision, skin tissue residing in front of the distal tip is ablated prior to encountering the distal tip; and
control and processing circuitry operably coupled to the laser source, the control and processing circuitry comprising at least one processor and associated memory, the memory comprising instructions executable by the at least one processor for performing operations comprising:
   controlling the laser source such that during formation of the incision by translation of the laser pulse delivery tool, the laser pulses are prevented from being delivered to the distal tip when the ablation direction is not aligned with a direction of travel of the laser pulse delivery tool.

In some example implementations of the system, the optical waveguide is a first optical waveguide, the distal tip is a first distal tip, the contact location is a first contact location, and the ablation location is a first ablation location, and the ablation direction is a first ablation direction;
the laser pulse delivery tool further comprising a second optical waveguide extending from the moveable support, the second optical waveguide comprising a second distal tip that resides adjacent to the first distal tip;
the second optical waveguide being in optical communication with the laser source;
the first distal tip being configured such that the first ablation location that resides laterally adjacent to the contact location resides on an opposing side of the first optical waveguide relative to the second optical waveguide;
the second distal tip being configured such that when the second distal tip contacts the skin tissue at a second contact location, laser pulses emerging from the second distal tip are directed at a second ablation location that resides laterally adjacent to the second contact location, on an opposing side of the second optical waveguide relative to the first optical waveguide, for ablating skin tissue adjacent to the second contact location, the second ablation location thereby residing, laterally relative to the second distal tip, along a second ablation direction, such that when the laser pulse delivery tool is translated in the second ablation direction during formation of the incision, skin tissue residing in front of the second distal tip is ablated prior to encountering the second distal tip;
wherein the control and processing circuitry is further configured to control the laser source such that when the laser pulse delivery tool is translated along the first ablation direction, the laser pulses are delivered to the first optical waveguide and prevented from being delivered to the second optical waveguide, and such that when the laser pulse delivery tool is translated along the second ablation direction, the laser pulses are delivered to the second optical waveguide and prevented from being delivered to the first optical waveguide;
such that when the laser pulse delivery tool is translated in the first ablation direction during formation of the incision, skin tissue residing in front of the first distal tip is ablated prior to encountering the first distal tip, and such that when the laser pulse delivery tool is translated in the second ablation direction during formation of the incision, skin tissue residing in front of the second distal tip is ablated prior to encountering the second distal tip.

In another aspect, there is provided a system for forming an incision, the system comprising:
a laser system configured to generate and deliver laser pulses, the laser system comprising a laser pulse delivery tool having a functional distal region, the functional distal region comprising an optical waveguide for delivering the laser pulses, wherein the laser system is configured such that the laser pulses delivered by the optical waveguide are capable of ablating skin tissue when a distal tip of the optical waveguide is contacted with the skin tissue;
a sensor configured to generate signals dependent on a force applied by the skin tissue to the distal tip during translation of the laser pulse delivery tool and formation of the incision; and
control and processing circuitry operably coupled to the sensor and the laser system, the control and processing circuitry comprising at least one processor and associated memory, the memory comprising instructions executable by the at least one processor for performing operations comprising:
   employing the signals to generate a feedback signal for controlling the laser system such that a rate of ablation of the skin tissue is varied according to the feedback signal to limit the force applied by the skin tissue to the distal tip, to prevent substantial force-induced scar tissue formation.

In some example implementations of the system, the sensor is configured such that the signals are dependent on an amount to deflection of the optical waveguide.

In some example implementations of the system, the sensor is configured such that the signals are dependent on an amount of pressure applied to the distal tip by the skin tissue.

In some example implementations of the system, the control and processing circuitry is configured generate alert when a threshold associated with the signals is exceeded.

In another aspect, there is provided a system for forming an incision, the system comprising:
a laser system configured to generate and deliver laser pulses, the laser system comprising a laser pulse delivery tool having a functional distal region, the functional distal region comprising an optical waveguide for delivering the laser pulses, wherein the laser system is configured such that the laser pulses delivered by the optical waveguide are capable of ablating skin tissue when a distal tip of the optical waveguide is contacted with the skin tissue;
a thermal sensor configured to generate signals dependent on a local temperature of the skin tissue proximal to the distal tip of the optical waveguide during translation of the laser pulse delivery tool and formation of the incision; and
control and processing circuitry operably coupled to the thermal sensor and the laser system, the control and processing circuitry comprising at least one processor and associated memory, the memory comprising instructions executable by the at least one processor for performing operations comprising:
   employing the signals to control a pulse repetition rate of the laser system in real-time to prevent or reduce thermally-induced scarring of residual non-ablated skin tissue.

In some example implementations of the system, the thermal sensor is remote from the laser pulse delivery tool.

In some example implementations of the system, the thermal sensor is secured to the laser pulse delivery tool. The thermal sensor may be configured such that when the laser pulse delivery tool is translated to form the incision, the thermal sensor interrogates an adjacent tissue region that is spatially adjacent, along an incision path, relative to an irradiated region that is irradiated by the laser pulses.

In some example implementations of the system, the laser system is configured to generate the laser pulses with a wavelength such that absorption of a given laser pulse by the skin tissue is predominantly due to excitation of vibrational modes of one or more constituents of the skin tissue;
the laser system being configured to respectively generate and deliver the laser pulses such that when a given volume of skin tissue is irradiated by contact of the distal tip of the optical waveguide with the skin tissue:
   a pulse duration is shorter than a first time duration required for thermal diffusion out of the laser-irradiated volume of skin tissue and shorter than a second time duration required for a thermally driven expansion of the laser-irradiated volume of skin tissue;
   the pulse duration and a pulse fluence result in a peak pulse intensity below a threshold for ionization-driven ablation to occur within the laser-irradiated volume of skin tissue; and
   the pulse fluence is sufficiently high such that the laser-irradiated volume of skin tissue is ablated and such that any residual energy is insufficient to induce substantial laser-induced scar tissue formation.

In another aspect, there is provided a system for forming an incision, the system comprising:
a laser system configured to generate and deliver laser pulses, the laser system comprising a laser pulse delivery tool having a functional distal region, the functional distal region comprising an optical waveguide for delivering the laser pulses, wherein the laser system is configured such that the laser pulses delivered by the optical waveguide are capable of ablating skin tissue when a distal tip of the optical waveguide is contacted with the skin tissue;
a sensor configured to generate signals suitable for determining a translation speed of the laser pulse delivery tool relative to the incision during translation of the laser pulse delivery tool and formation of the incision; and
control and processing circuitry operably coupled to the sensor and the laser system, the control and processing circuitry comprising at least one processor and associated memory, the memory comprising instructions executable by the at least one processor for performing operations comprising:
   employing the signals to control a pulse repetition rate of the laser system in real-time to prevent or reduce thermally-induced scarring of residual non-ablated skin tissue.

In some example implementations of the system, the sensor is remote from the laser pulse delivery tool.

In some example implementations of the system, the sensor is secured to the laser pulse delivery tool. The sensor may be an inertial sensor or a position sensing sensor.

In some example implementations, the system further comprises an incision guide for guiding translation of the laser pulse delivery tool, wherein the incision guide is configured to be removably secured to the skin and to guide translation of the laser pulse delivery tool along a prescribed incision path, the incision guide comprising one or more features suitable for generating a change in the signals detected by the sensor when the laser pulse delivery tool is translated relative to the incision guide and passes proximal to each features.

In some example implementations of the system, the laser system is configured to generate the laser pulses with a wavelength such that absorption of a given laser pulse by the skin tissue is predominantly due to excitation of vibrational modes of one or more constituents of the skin tissue;
the laser system being configured to respectively generate and deliver the laser pulses such that when a given volume of skin tissue is irradiated by contact of the distal tip of the optical waveguide with the skin tissue:
   a pulse duration is shorter than a first time duration required for thermal diffusion out of the laser-irradiated volume of skin tissue and shorter than a second time duration required for a thermally driven expansion of the laser-irradiated volume of skin tissue;
   the pulse duration and a pulse fluence result in a peak pulse intensity below a threshold for ionization-driven ablation to occur within the laser-irradiated volume of skin tissue; and
   the pulse fluence is sufficiently high such that the laser-irradiated volume of skin tissue is ablated and such that any residual energy is insufficient to induce substantial laser-induced scar tissue formation.

In another aspect, there is provided a system for forming an incision, the system comprising:
a laser pulse delivery tool comprising:
   a support; and
   an optical waveguide extending from the support, the optical waveguide comprising a distal tip;
a laser source in optical communication with the laser pulse delivery tool for delivering laser pulses to the optical waveguide, such that the laser pulses are directed through the optical waveguide to the distal tip, the laser pulses being suitable for ablating skin tissue when the distal tip is contacted with the skin tissue and the laser pulses are delivered to the skin tissue through the distal tip;
a support substrate comprising a first elongate substrate portion and a second elongate substrate portion, the support substrate being configured to be removably attachable to a skin surface such that the first elongate substrate portion and the second elongate substrate portion are provided in a spaced relationship, and such that a skin region is accessible between the first elongate substrate portion and the second elongate substrate portion; and
a rigid guide structure configured to be removably and rigidly attachable to the support substrate such that a separation between the first elongate substrate portion and the second elongate substrate portion is fixed by the rigid guide structure, the rigid guide structure being configured to guide translation of the laser pulse delivery tool along a prescribed incision path residing within the skin region between the first elongate substrate portion and the second elongate substrate portion for forming the incision while remaining fixed to the support substrate.

In some example implementations of the system, the laser pulse delivery tool comprises first guiding features that engage with corresponding second guide features of the rigid guide structure to permit and guide translation of the laser pulse delivery tool relative to the rigid guide structure for forming the incision.

In some example implementations, the system further comprises a tool carriage configured to support the laser pulse delivery tool relative to the rigid guide structure and facilitate translation of the laser pulse delivery tool relative to the rigid guide structure, the tool carriage comprising first guiding features that engage with corresponding second guiding features of the rigid guide structure to permit and guide translation of the tool carriage relative to the rigid guide structure.

In some example implementations of the system, the system further comprises an alignment member for defining an initial spacing between the first elongate substrate portion and the second elongate substrate portion during attachment of the first elongate substrate portion and the second elongate substrate portion to the skin surface, the alignment member being removable after attachment of the first elongate substrate portion and the second elongate substrate portion to the skin surface. The alignment member may be configured such that the initial spacing is selectable prior to attachment of the first elongate substrate portion and the second elongate substrate portion to the skin surface.

In some example implementations of the system, the support substrate and the rigid guide structure comprise respective engagement features such that an intraoperative separation between the first elongate substrate portion and the second elongate substrate portion is enforced by the rigid guide structure. The rigid guide structure may be configured such that the intraoperative separation between the first elongate substrate portion and the second elongate substrate portion is controllable after attachment of the rigid guide structure to the support substrate, thereby permitting intraoperative tuning of an amount of tension applied to the skin region.

The system may further comprise an alignment member configured to engage with the support substrate for defining an initial separation between the first elongate substrate portion and the second elongate substrate portion during attachment of the first elongate substrate portion and the second elongate substrate portion to the skin surface, the alignment member being removable after attachment of the first elongate substrate portion and the second elongate substrate portion to the skin surface. The initial separation may be different from the intraoperative separation, such a prescribed amount of tension is applied across the skin region after attachment of the rigid guide structure to the support substrate and during formation of the incision. The alignment member and the rigid guide structure may be configured such that a difference between the initial separation and the intraoperative separation results in an applied tension that is less than an elastic deformation limit of skin. The alignment member and the rigid guide structure may be configured such that a difference between the initial separation and the intraoperative separation results in an applied tension, within the incision, during formation of the incision, that remains within an elastic deformation limit skin tissue.

The alignment member may be configured such that the initial separation is selectable prior to attachment of the first elongate substrate portion and the second elongate substrate portion to the skin surface for controlling an amount of tension applied within the skin region during formation of the incision.

In another aspect, there is provided a system for forming an incision, the system comprising:
a laser pulse delivery tool comprising:
   a support; and
   an optical waveguide extending from the support, the optical waveguide comprising a distal tip;
a laser source in optical communication with the laser pulse delivery tool for delivering laser pulses to the optical waveguide, such that the laser pulses are directed through the optical waveguide to the distal tip, the laser pulses being suitable for ablating skin tissue when the distal tip is contacted with the skin tissue and the laser pulses are delivered to the skin tissue through the distal tip;
a support substrate comprising a first elongate substrate portion and a second elongate substrate portion, the support substrate being configured to be removably attachable to a skin surface such that the first elongate substrate portion and the second elongate substrate portion are provided in a spaced relationship, such that the skin surface is accessible between the first elongate substrate portion and the second elongate substrate portion;
a guide structure configured to be removably attachable to the support substrate, the guide structure being further configured to receive and guide translation of the laser pulse delivery tool along a prescribed incision path residing between the first elongate substrate portion and the second elongate substrate portion for forming the incision;
the laser pulse delivery tool comprising a depth control mechanism for controlling a depth of the distal tip relative to the skin surface when the laser pulse delivery tool is engaged with the guide structure; and
control and processing circuitry operably coupled to the laser pulse delivery tool, the control and processing circuitry comprising at least one processor and associated memory, the memory comprising instructions executable by the at least one processor for performing operations comprising:
   controlling the depth control mechanism such that contact of the distal tip with skin tissue within the incision is maintained as a depth of the incision increases during formation of the incision.

In some example implementations of the system, the depth control mechanism is controlled in an open-loop configuration according to control criteria dependent on a number of passes of the laser pulse delivery tool along the prescribed incision path.

In some example implementations of the system, at least one of the laser pulse delivery tool and the guide structure comprises a sensor for detecting a number of passes of the laser pulse delivery tool during formation of the incision, wherein the control and processing circuitry is operably coupled to the sensor.

In some example implementations, the system further comprises a sensor for sensing signals dependent on the depth of the incision, and wherein the depth control mechanism is controlled in a closed-loop configuration according to control criteria that employs the signals from the sensor.

In some example implementations, the system further comprises a sensor for sensing signals dependent on a force applied to the distal tip by the skin tissue during translation of the laser pulse delivery tool, and wherein the depth control mechanism is controlled, based on the signals, to maintain contact between the distal tip and the skin tissue. The sensor may be configured such that the signals are dependent on an amount to deflection of the optical waveguide. The sensor may be configured such that the signals are dependent on an amount of pressure applied to the distal tip by the skin tissue. The depth control mechanism may be controlled, based on the signals, to limit the force within a range that is suitable for maintaining contact between the distal tip and the skin tissue while preventing substantial force-induced scar tissue formation.

The depth control mechanism may be controlled, based on the signals, to limit the force within an elastic deformation limit of the skin tissue.

In some example implementations of the system, the laser pulse delivery tool further comprises a sensor for sensing signals dependent on a spatial offset, in a depth direction, between the distal tip and a bottom of the incision, and wherein the depth control mechanism is controlled in a closed-loop configuration according to control criteria that employs the signals from the sensor.

In another aspect, there is provided a system for forming an incision, the system comprising:
a laser system configured to generate laser pulses;
a scanning system configured to direct the laser pulses onto a skin surface of a subject and to scan the laser pulses relative to the skin surface for forming the incision;
a tensioning mechanism configured to apply tension across the incision during formation of the incision;
the laser system being configured to generate the laser pulses with a wavelength such that absorption of the laser pulses by skin tissue is predominantly due to excitation of vibrational modes of water within the skin tissue;
the laser system and the scanning system being configured to respectively generate and deliver the laser pulses such that:
   a pulse duration is shorter than a first time duration required for thermal diffusion out of a laser irradiated volume of skin tissue and shorter than a second time duration required for a thermally driven expansion of the laser irradiated volume of skin tissue;
   the pulse duration and a pulse fluence result in a peak pulse intensity below a threshold for ionization-driven ablation to occur within the laser irradiated volume of skin tissue; and
   the pulse fluence is sufficiently high such that the laser irradiated volume of skin tissue is ablated and such that any residual energy is insufficient to induce substantial laser-induced scar tissue formation;
wherein the tensioning mechanism is configured to apply to sufficient tension to maintain a line-of-sight between an output aperture of the scanning system and a distal region of the incision during formation of the incision while preventing substantial tension-induced scar tissue formation.

In another aspect, there is provided a system for controlling formation of an incision, the system comprising:
a tensioning device contactable with a skin surface for controllably applying tension across a skin region;
an imaging device positionable relative to the tensioning device such that when the incision is formed in the skin region and a distal trough of the incision is exposed under application of tension by the tensioning device, the distal trough resides within a field of view of the imaging device;
control and processing circuitry comprising at least one processor and associated memory, the memory comprising instructions executable by the at least one processor for performing operations comprising:
   while controlling the tensioning device to vary the tension applied across the skin region:
      controlling the imaging device to acquire a plurality of images corresponding to different amounts of applied tension;
      processing the plurality of images to generate incision measures comprising at least (i) a first measure characterizing a presence or absence of the distal trough of the incision and (ii) a second measure characterizing elasticity of deformation of an exposed internal cutaneous tissue surface to the tension; and
   employing the incision measures to control the tensioning device to apply tension such that the distal trough of the incision is exposed and such that deformation of the exposed internal cutaneous surface by the tension is elastic, thereby preventing or reducing formation deformation-induced generation of scar tissue.

In another aspect, there is provided a device for retraction of an incision, the device comprising:
a first elongate member and a second elongate member, the first elongate member and the second elongate member being removably securable relative to a skin surface such that the first elongate member and the second elongate member reside adjacent one another, and such that the incision resides between the first elongate member and the second elongate member;
each of the first elongate member and the second elongate member comprising a respective incision edge protection feature located such that when the first elongate member and the second elongate member reside adjacent one another, each incision edge protection feature is insertable into the incision, such that each incision edge protection feature resides adjacent to a respective incision edge; and
the device further comprising a retraction mechanism connecting the first elongate member and the second elongate member, the retraction mechanism being operable to increase a separation between the first elongate member and the second elongate member, thereby retracting the incision through application of tension across the skin surface while avoiding substantial deformation of the incision edges by the incision edge protection features.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1A plots the threshold fluence, as a function of wavelength, for ionization and for ablation via the PIRL impulsive heat deposition mechanism.

FIG. 1B illustrates an example system for forming an incision in tissue using a laser pulse delivery tool having a distal optical waveguide for beam delivery.

FIGS. 2 and 3 illustrate the use of a guideline for the formation of an incision in tissue using a laser pulse delivery tool having a distal optical waveguide for beam delivery.

FIGS. 4A-4D illustrate various example optical waveguides for the delivery of laser pulses to form an incision.

FIGS. 5 and 6 illustrate example optical fiber arrays for the ablation of tissue in a forward direction along the path of travel during the formation of an incision.

FIGS. 7A and 7B illustrate the deflection of an optical waveguide due to applied pressure while forming an incision.

FIGS. 8A-8C illustrate an example support substrate for use in supporting one or more structures during or after the formation of an incision.

FIGS. 9A-9F illustrate an example tension control structure for applying tension during the formation of an incision.

FIG. 10 illustrates an example system that employs a tension control structure to applying controlled tension during the formation of an incision.

FIGS. 11A-11B illustrate an example guide structure for guiding translation of a laser pulse delivery tool during formation of an incision.

FIGS. 12A-12B illustrate the use of an example guide structure to guide translation of a laser pulse delivery tool during formation of an incision.

FIGS. 13A-13H illustrate an example guide structure for guiding translation of a laser pulse delivery tool during formation of an incision while applying tension across the incision.

FIGS. 14A-14B illustrate an example robotic system for forming an incision, the robotic system including a distal head having an optical waveguide for laser beam delivery.

FIGS. 15A-15B illustrate an example robotic system for forming an incision, the robotic system including a distal head having one or more optical components for free-space laser beam delivery.

FIGS. 16A-16C illustrate an example robotic system for forming an incision, the robotic system including a tensioning mechanism for the controlled application of tension during the formation of the incision.

FIGS. 17-21 illustrate an example implementation of an incision retraction structure 600 that can be employed to protect the edges of an incision when opening and closing an incision.

FIGS. 22A-22D illustrate an example implementation of a retraction mechanism.

FIGS. 22E-22H illustrate an example implementation of a motorized retraction mechanism.

FIGS. 23-27 illustrate an example incision retraction structure suitable for forming an incision opening facilitating the insertion of a cylindrical tool.

FIGS. 28 and 29 illustrate the effect of incision length on the generation of inelastic deformation at the incision edges.

FIGS. 30A and 30B present results from finite element method (FEM) analysis illustrating the effect of incision widening on the spatial stress distribution within the incision corner regions.

FIGS. 31A and 31B presents example parameters of bilinear elastic tissue deformation model.

FIGS. 32A and 32B present results from two finite element simulations with different incision lengths.

FIG. 33A plots the simulated dependence of incision corner stress on incision opening width for an example incision length of 99 mm.

FIG. 33B plots the dependence of incision corner stress on incision width for several different incision lengths.

FIG. 33C plots a relationship between maximum incision width and incision length.

FIGS. 34 and 35A-35C illustrate an example incision closure device.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

As noted above, when conventional methods are employed to form incisions, microscopic damage caused at the edges of the incision lead to scar tissue formation. Indeed, incisions formed mechanically via the use of a conventional scalpel result in tissue necrosis and subsequent scar tissue formation due to inelastic deformation of tissue. Specifically, forces applied during the formation of the incision damage tissue by distorting cell membranes, which triggers a healing response cascade. This response involves signaling proteins for fibroblast formation, which occurs throughout the depth of the tissue that has undergone deformation. Indeed, the inelastic tissue deformation caused by the use of a scalpel induces proteins/factors that trigger fibroblast formation and associated cell division, resulting in collateral damage that can typically occurs over a region extending 400 microns to 1 mm from the edge of the incision. Scar tissue that is generated within this region arises from the spatially uncorrelated formation of fibroblast bundles/fibrils that create a tissue mesh to close the wound. While some researchers and clinicians have turned to surgical lasers in an attempt to avoid the scar formation associated with conventional scalpels, most surgical laser systems, such as those that employ CO₂ and Er laser systems, rely on the local deposition of heat and result in scar formation due to thermal damage and damage from shock waves.

In contrast, some cold laser ablative methods that do not rely on heat have been shown to hold promise for scar-free incisions. Such cold laser ablative methods employ laser pulses that result in the deposition of laser energy into the desired ablation process for free boundary conditions or into targeted tissue disruption for direct contact, with negligible transfer of energy in the form of heat to the adjacent tissue. The cutting or tissue disruption process is fully contained in the desired target volume for a cold cutting process. In the case of a free boundary or surface cutting, the deposited energy is transduced into translational energy of the constituent molecules and particulates in which the tissue disruption leads to mass removal or ablation. The energy goes into translational motions rather than transfer of heat to surrounding tissue. In the case of direct contact where the tip of optical delivery system contacts skin, the material removal does not physically remove the energy from the tissue, the energy is consumed in tissue disruption. In both cases, the energy derived to remove tissue or result in tissue disruption does not resulting in heating of the adjacent tissue and therefore is referred to as a cold cutting process.

For example, picosecond infrared laser (PIRL) systems have been shown to facilitate the formation of incisions with reduced scar tissue formation in experiments involving in small animals. Unlike conventional surgical laser methods that rely on thermal ablation as in electrosurgery, the PIRL ablation mechanism involves the delivery of short infrared laser pulses having wavelengths specifically tuned to vibrational modes of water. The pulse duration of PIRL laser pulses is sufficiently short to drive ablation faster than the timescales associated with thermal and acoustic transport to remove or disrupt tissue in a so-called cold ablation process with respect to surrounding tissue. The energy above the ablation threshold is fully confined to the targeted tissue, thus avoiding collateral damage due to heat and shock wave formation to surrounding tissue, while also being sufficiently long to avoid the ionizing radiation effects of plasma formation.

The absorption of PIRL pulses by tissue results in superheating on picosecond timescales, with nanometer-sized nucleation sites that drive the ablative phase transition and avoids cavitation and associated shock wave induced damage. The strong acoustic attenuation at the 100 GHz frequency range associated with PIRL laser pulses further assists in ensuring that the absorbed energy results in tissue ablation on timescales much faster than conventional heat transfer can damage adjacent tissue of the targeted area. The cold ablative mechanism of PIRL is also referred to as desorption by impulsive vibrational excitation (DIVE).

Despite the promise of PIRL, the present inventors encountered numerous problems when attempting to implement PIRL for scar-free incisions in human skin. In particular, the present inventors found that despite the inherent cold ablative capability of PIRL, the ability to employ PIRL for the formation of substantially scar-free incisions in human skin was often prohibited by the absence of suitable temporal and spatial control of the delivery of laser pulses. For example, as a consequence of the higher thickness of human skin compared to that of small animals (in which initial PIRL experiments had been performed), it is typically necessary to perform numerous passes of a PIRL laser beam in order to generate a sufficiently deep incision, and this requirement can introduce several problems in when attempting to form incisions in human skin using PIRL.

One problem associated with multi-pass PIRL-based incisions in skin is the challenge in maintaining a sufficiently high degree of spatial alignment among multiple passes of the laser beam in order to form a narrow incision. In principle, PIRL is capable of performing tissue ablation at the single-cell level, with ablation occurring with a lateral extent of approximately 10 microns or less, depending on the beam size on the target (e.g., a lower limit is determined by the Rayleigh criteria) with little or no residual collateral damage and scar tissue formation. Typically spot sizes of 100 micron are used for more convenient for efficient cutting at practical pulse energies, while still maintaining tight enough focus for reaching ablation or cutting thresholds. Another advantage of larger spot sizes is greater depth of field, i.e. longer working distance for the beam. However, the present inventors found that in practice, it can be very difficult to maintain the requisite level of positional accuracy over multiple passes of the PIRL laser beam, especially when using a manual laser delivery tool, with the consequence that multi-pass PIRL-based incisions, when formed without suitable feedback or controls, can often be significantly wider (and more visible post-operatively) than the initial incision that is formed from the first pass of the PIRL beam. Accordingly, some example embodiments of the present disclosure provide methods and associated apparatus that facilitate lateral alignment of a PIRL-based laser-beam during multiple scans during the formation of an incision.

Another problem that was encountered by the inventors during their attempts to implement multi-pass PIRL-based ablation for the formation of incisions in human skin was the need to control the temporal and spatial delivery of the laser pulses along the incision path. For example, when a handheld laser delivery tool was used to deliver the laser pulses to the skin, with the handheld laser delivery tool being moved by an operator along the incision path, variations in the rate of translation of the tool were found to lead to the local accumulation of residual heat, thereby risking heat-induced tissue necrosis and scar tissue formation. Moreover, variations in the rate of the translation of the laser delivery tool also generated local variations in the depth of the incision along the incision path due to different dwell times of the tool along the incision path. Such effects can be pronounced, for example, at the ends of the incision, where the laser delivery tool is typically slowed to a stop before being moved in the opposite direction along the incision path to initiate the next pass of the laser beam. These variations in incision depth can be problematic, for example, in the case of a fiber-optic-based delivery tool, as the depth variations can locally “catch” the distal end of the optical fiber during translation of the tool along the incision path and can result in scar tissue formation due to inelastic deformation of the tissue.

The present inventors also found that even though the individual ablation events associated with the delivery of PIRL pulses could facilitate the direct ablation of skin tissue without causing collateral damage to the residual tissue, the need to maintain open line-of-sight to the bottom (trough) of the incision during the multiple passes of the PIRL beam could result in scar tissue formation. Indeed, the present inventors found that the need to maintain a line-of-sight path to the bottom of the incision during the multiple passes of the PIRL beam, as the incision deepened, often required the application of tension across the incision, and that the application of such tension, if not controlled, could in some cases result in the application of an amount of strain that exceeded the elastic deformation limit of the tissue within the incision. The resulting inelastic strain deforms the cells within the tissue and by definition constitutes tissue damage that leads to excessive scar tissue formation beyond the edge defining the physical dimensions of the cut. It is beneficial to eliminate or minimize as much as possible this transverse inelastic deformation in the cutting process to avoid scar tissue formation.

It was also found that high levels of tension are typically applied within the incision when opening the incision after its formation to permit the passage of surgical tools and/or to permit the observation of underlying tissue structures. In particular, as explained in further detail below, the present inventors found that the as the incision was opened after its formation, the strain could be amplified at or near the incision vertices, potentially leading to a local strain the exceeds the elastic deformation limit of the tissue and the associated generation of scar tissue.

Furthermore, the present inventors found that even after forming a precise and clean incision within skin using a scanned PIRL laser beam and ensuring the that the tension applied to the incision was sufficiently low to maintain the intra-incision strain below the elastic deformation limit, it could be challenging to rejoin the incision edges (e.g. using sutures) after a surgical procedure without applying strain to the incision edges that exceeds the elastic deformation limit of the tissue. In such cases, even though care was taken during the incision formation process, the process of rejoining the incision edges to close the wound could nonetheless induce tissue necrosis and scar tissue formation.

The present inventors thus set out to solve the aforementioned problems and develop solutions that would facilitate the practical and clinical application of PIRL for the formation of incisions with little or no visible scar tissue. Accordingly, various example embodiments of the present disclosure provide solutions that enable the use of PIRL for the formation of incisions within skin tissue, using multiple passes of a PIRL laser beam, while facilitating precise spatial alignment of successive passes of the PIRL laser beam along the incision, and without exceeding the elastic deformation limit of tissue residing within the incision. The present inventors have found that when the present example systems, methods and apparatus are employed, incisions can be formed within human skin with little or no scarring visible to the unaided eye.

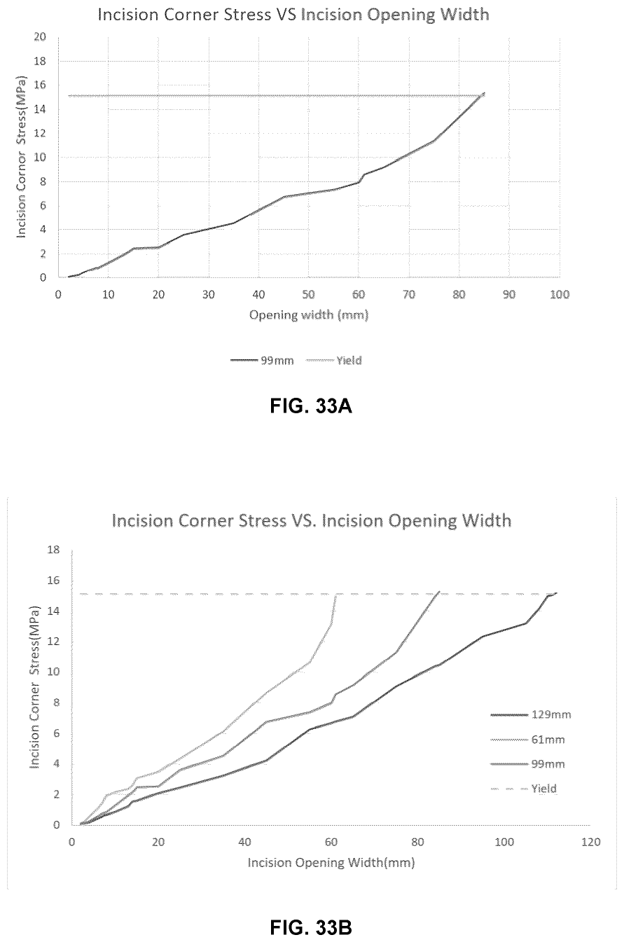

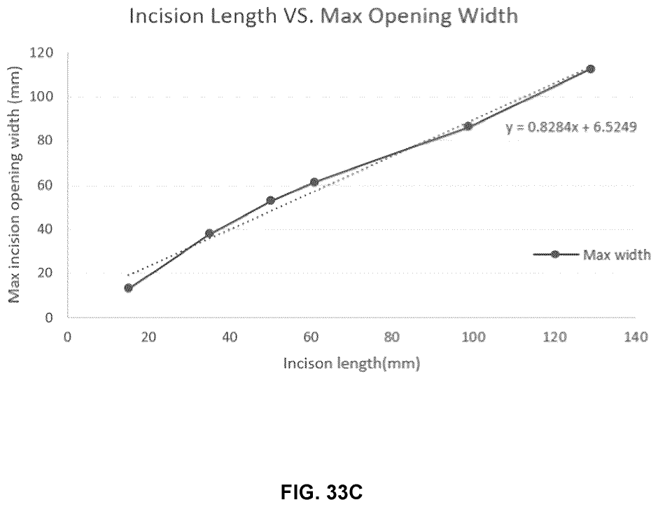

The elastic deformation limit of the tissue is approximately 18-20 MPa in the direction parallel to the incision and approximately 13-15 MPa in the direction perpendicular to the incision. Incision edges will experience tension parallel to the incision and the incision corner will experience tension perpendicular to the incision direction. If tension stress that is applied to the skin incision edges or corner exceeds the corresponding ultimate stress limit, plastic deformation will occur to the skin tissue. Quantitative estimates of the maximum width of an incision that does not exceed the elastic deformation limit are provided in the results simulation results shown in FIG. 33A. For example, in the case of an incision with a length of 50 mm, the incision can widen to approximately 45 mm before exceeding the elastic deformation limit.

In various example implementations of the present disclosure, systems, methods and apparatus are provided for employing PIRL to provide laser pulses that facilitate the ablation of tissue while reducing or preventing substantial generation of scar tissue within residual tissue surrounding the ablation site. In the following disclosure, the phrase “scar-free”, when employed to refer to the formation and/or closure of an incision, means absent of visible scarring when viewed by the unaided eye. While some example implementations of the present disclosure may result in scar-free incision formation and healing, in other example implementations, some visible scarring may result, albeit in an amount that is significantly less than that which would have formed using conventional approaches to incision formation, closure and/or healing. Accordingly, many example embodiments of the present disclosure facilitate control over parameters that can influence scar tissue formation, thereby enabling the reduction, minimization, or prevention of the triggering of scarring processes during incision formation, closure and/or healing, resulting in the reduction, minimization, or prevention (elimination) of visible scars, or, for example, internal adhesions due to scar tissue. For example, in the case of keloid scar sufferers, example embodiments of the present disclosure may be beneficial in facilitating the formation, closure and/or healing a keloid scar with reduced scar tissue formation as compared to conventional approaches.

In the following example embodiments, PIRL laser pulses are employed that are sufficiently short to drive ablation faster than the timescales associated with thermal and acoustic transport, thus avoiding damage due to heat and shock wave formation, while also being sufficiently long to avoid the ionizing radiation effects of plasma formation. PIRL pulses are provided with a wavelength selected such that absorption of the laser pulses by tissue is predominantly due to excitation of vibrational modes of one or more constituents of the tissue, such as water. Suitable wavelength ranges for PIRL laser pulses therefore include 2.7-3.3 μm, 5.9-6.1 μm and 1.8-2.0 μm. Future developments in high energy and short pulsed laser sources will enable PIRL cutting by targeting vibrational absorption in target molecules between 2-20 um.

For example, the PIRL laser pulse wavelength may be selected to overlap with, or reside proximal to, a strong peak in the vibrational spectrum of a constituent of the tissue, such as the OH-stretch region of H₂O. Such vibrational modes quickly absorb the electromagnetic radiation and may effectively localize optical energy to micron scale deep sections of the exposed tissue. In the case of water, maximum absorption for vibrational modes occurs between about 2.7-3.33 μm, where broad peak in the absorption spectrum corresponds to the OH-stretching vibrational modes of liquid water molecules. The spectrum also shows the resonance conditions between the OH-stretch and other vibrational modes such as the OH bend and Intermolecular modes. Other absorption peaks, for example, at approximately 1.9 μm or approximately 6 μm, may alternatively be employed.

In various example embodiments, PIRL pulses are generated and delivered such that when a given volume of skin tissue is irradiated, the pulse duration is shorter than (i) the time duration required for thermal diffusion out of the laser-irradiated volume of skin tissue, and (i) the time duration required for a thermally driven expansion of the laser-irradiated volume of skin tissue. The skilled artisan will be able to determine a suitable pulse duration for PIRL pulses for a given pulse wavelength and absorption depth in tissue. In general, for a given PIRL laser pulse wavelength that is selected according to the aforementioned criterion (absorption of the laser pulses by tissue is predominantly due to excitation of vibrational modes of one or more constituents of the tissue), the known properties of the tissue, such as the absorption depth of the laser pulses, thermal diffusion constant, and the speed of sound, may be employed to calculate a suitable PIRL pulse duration that satisfies criteria (i) and (ii) above. Alternatively or additionally, experiments may be performed to determine a suitable laser pulse duration that satisfies criteria (i) and (ii).

For example, in the case of ablating tissue with a laser wavelength of 3 μm, for which the absorption depth is approximately 1 μm, the maximum pulse duration can be calculated based on the ratio of absorption depth to speed of sound, 1730 m/sec. Or t=a/v=10⁻⁶ m/1.730×10³ m/s=5.78×10⁻¹⁰ sec, approximately 600 ps (e.g. see Duck, F. A., Physical Properties of Tissue, Academic Press, London, 1990, and Duck, F. A., Propagation of Sound Through Tissue, in “The Safe Use of Ultrasound in Medical Diagnosis”, ter Haar G and Duck, F. A, Eds., British Institute of Radiology, London, 2000, pp. 4-15).

Different tissue will have different absorption depth at a given wavelength, example bone, brain and skin. Around the OH-stretching band, the absorption of the tissue is dominated by the water content. For skin, the water content can vary between the surface and deeper layers. In general, the absorption depth will be longer than pure water. At a wavelength of 2.95 μm, the absorption depth of pure water is close to 0.7 μm, and given the variance in the high concentration of water in the skin, along with other OH-stretching modes in the tissue, the absorption depth of skin is thus approximately 1-2 μm at this wavelength. If the wavelength of the laser is shifted, e.g. to a wavelength of 2.75 μm, then the absorption depth of the light increases by a factor of about 3 according to the change in the absorption spectrum of the OH-stretch. (see, for example, Diaci, J., J. Laser and Health Acad. 2012, 1-13 (2012).

In another example in which skin tissue is ablated using a laser wavelength of 6 m, for which the absorption depth is approximately 100 m, the pulse duration should be chosen as shorter than 100 μm/1.753×10³=57 ns.

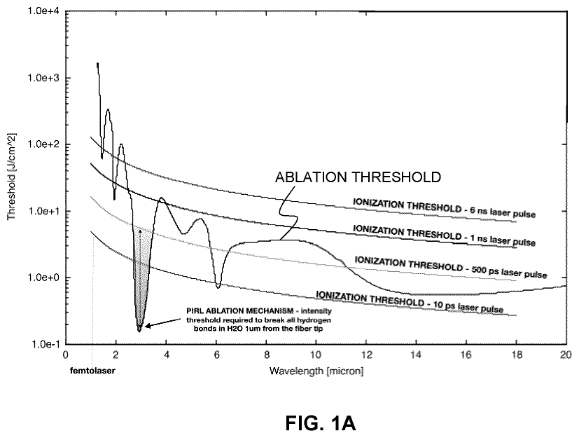

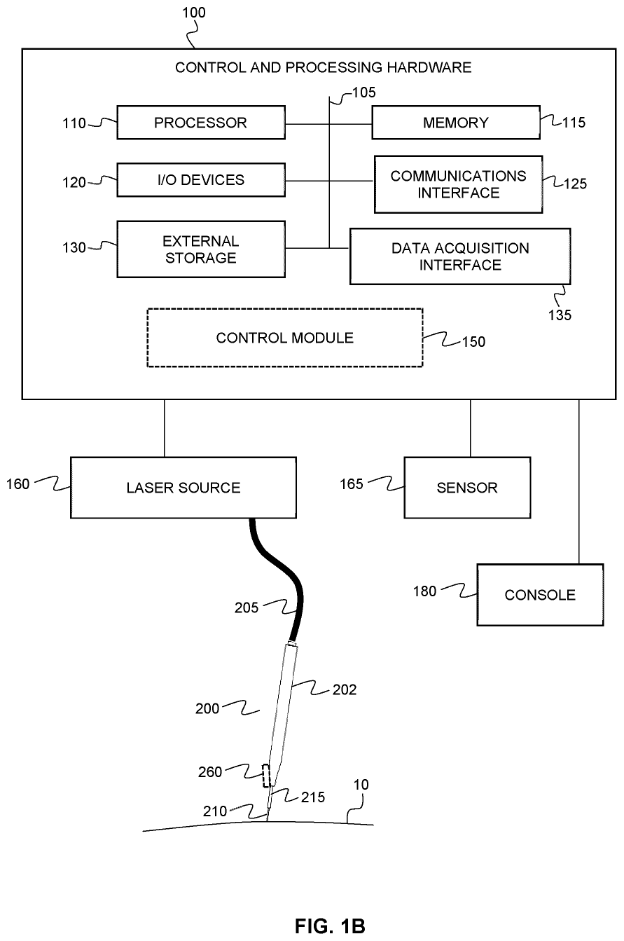

The pulse duration and pulse fluence are also selected such that a peak pulse intensity is bel ow a threshold for ionization-driven ablation to occur within the laser-irradiated volume of tissue. For example, for a given pulse duration, a suitable upper limit of the pulse fluence may be determined to avoid the threshold for ionization-driven ablation. In the example case of human skin tissue, at a laser wavelength 3 μm, the maximum fluence values for avoiding ionization-driven ablation, for pulse durations of 10 ps, 500 ps, and 1 ns, are approximately 1.5 J/cm², 5.5 J/cm², and 17 J/cm², respectively, as shown in the FIG. 1A.

Furthermore, in order to achieve PIRL-based ablation of tissue for laser pulses that satisfy the preceding criteria involving wavelength, pulse duration and pulse fluence, the laser pulses should be provided with a sufficient pulse fluence to achieve a threshold energy density for PIRL ablation, as shown, for example, by the ablation threshold identified in FIG. 1A. For example, the pulse fluence that is delivered to the tissue should be sufficiently high to that the energy deposited in the irradiated volume is sufficient to heat the contents of the volume up to its vaporization temperature including the enthalpy of vaporization.

For example, if the beam is focused to 200 μm (or a 200 μm fiber is used in contact) and ablates a volume of ˜1 μm deep×π(100 μm)² the mass of the ablated volume is 3.1×10⁻⁸ g in the case of water and 3.4×10⁻⁸ g in the case of skin (which has a density of 1.15 g/cm³). The energy required to raise the temperature of this volume of water from 20 to 100° C. and then vaporize the volume is approximately 80 μJ of energy, which corresponds to a fluence of 0.25 J/cm² for a 200 μm spot. This fluence defines the threshold for impulsive heat deposition to drive the phase transition without loss due to acoustic transport or thermal diffusion out of the excited zone. Higher degrees of superheating above this threshold leads to faster rates of vaporization with the excess energy going into translation energy or high exit velocity of the plume. To ensure the ensuing ablation process occurs in this limit, for highly scattering medium such as tissue which effectively decreases the incident intensity, typical excitation conditions used are 1 J/cm². The determination of a sufficient fluence for PIRL ablation can be made experimentally by varying the applied fluence, examining the resulting tissue ablation, and selecting an applied fluence value that provides a sufficient amount or degree of ablation.

Finally, during the process of ablating a tissue volume by a PIRL laser pulse, non-ablated tissue surrounding the ablated tissue volume may be heated due to the absorption of residual laser pulse energy. For example, a portion of the pulse energy that is absorbed beyond the absorption depth may be insufficient to cause ablation and will instead contribute to heating of this surrounding non-ablated tissue region. Accordingly, at least one of the pulse repetition rate and the scanning of the laser pulses may be controlled (e.g. via a shutter, laser modulator or other suitable means of rate of adjustment of the repetition rate of the laser system) such that residual laser pulse energy from multiple laser pulses absorbed at the same location, or from multiple laser pulses absorbed at spatially adjacent locations, does not lead to heat accumulation below the ablation threshold to a degree that would result in tissue necrosis and scar tissue formation.

For example, the pulse repetition rate and/or scanning of the pulses may be controlled such that the time interval between successive laser pulses directed to the same location, and/or to spatially adjacent locations (neighbouring) locations, exceeds the thermal diffusion time. For example, in the case of the formation an incision in human skin using PIRL laser pulses with a wavelength of 3 μm, the laser repetition rate and/or scanning of the pulses may be controlled such that this time interval between pulses being delivered to the same or to spatially adjacent regions is greater than the thermal diffusion time of skin under impulsive excitation, for example, 100-1000 μs, depending on the layer and amount of moisture present.

The preceding conditions for generating and delivering PIRL laser pulses to form incisions with reduced or minimized scar tissue formation, such as, for example, scar-free incisions, can be achieved using a wide variety of different laser systems. Non-limiting examples of suitable laser systems include a near-IR pumped optical parametric amplifier (e.g. emitting pulses with a duration in the hundreds of ps, such as 500 ps) tuned to a wavelength of approximately 2.95 μm, operating, for example, between 1-10 kHz with a pulse fluence greater than 0.5 mJ/pulse, delivered on target, for example, through a sapphire fiber optic (e.g. having a core diameter of 200 μm); and a Cr:ZnSe gain-switched laser, emitting ns pulses (e.g. 1.5 ns), tuned to a wavelength of approximately 2.7 μm, operating, for example, between 1-10 kHz, with a pulse fluence greater than >1 mJ/pulse, e.g. focused onto the surface by an imaging system.

Moreover, it will be understood that the laser pulses that are delivered to the tissue surface to form the incision may be delivered via an optical waveguide, such as an optical fiber tip, or via free space (e.g. focused by a focusing element through a free-space path onto the tissue).

In some example embodiments of the present disclosure, a handheld laser delivery tool (laser pulse delivery tool) is employed to deliver PIRL laser pulses for the formation of incisions in skin without generating substantial scar tissue within residual tissue surrounding the incision.

An example schematic of such a system is illustrated in FIG. 1B. Laser pulses are generated by a laser source 160, such as a PIRL laser source, and the laser pulses are delivered through an optical fiber 205 to a laser pulse delivery tool 200 (handpiece). An optical waveguide 210 forms a distal functional region of the laser pulse delivery tool 200, with the optical waveguide extending from a distal end of a main body portion 202 of the laser pulse delivery tool 200. As shown in the figure, a portion of the optical waveguide 210 may be cladded with a protective cladding or sheath 215. The optical waveguide 210 is employed to deliver the laser pulses to the tissue surface 10 to form the incision. The optical waveguide 210 may be a distal portion of the optical fiber 205. Alternatively, the optical waveguide 210 may be a separate structure to which the optical fiber 205 is optically coupled (e.g. within the main body 202 of the laser pulse delivery tool 200) for delivery of the PIRL pulses thereto. Various example structural forms of the optical waveguide 210 are described in detail below.

The laser source 160 is operatively coupled or connectable to control and processing hardware 100 for control thereof. The example control and processing hardware 100 may include a processor 110, a memory 115, a system bus 105, one or more input/output devices 120, and a plurality of optional additional devices such as communications interface 125, external storage 130, and a data acquisition interface 135. In one example implementation, a display (not shown) may be employed to provide a user interface for facilitating input to control the operation of the system 100. The display may be directly integrated into a control and processing device (for example, as an embedded display), or may be provided as an external device (for example, an external monitor).

The control and processing system 100 may include or be connectable to a console 180 that provides an interface for facilitating an operator to control the laser source 160. The console may include, for example, one or more input devices, such, but not limited to, a keypad, mouse, joystick, touchscreen, and may optionally include a display device.

The methods described herein, such as methods for controlling the pulse repetition rate of the laser source 160 or other example methods described below, can be implemented via processor 110 and/or memory 115. As shown in FIG. 1B, executable instructions represented as control module 150 are processed by control and processing hardware 100. Such executable instructions may be stored, for example, in the memory 115 and/or other internal storage.

The methods described herein can be partially implemented via hardware logic in processor 110 and partially using the instructions stored in memory 115. Some embodiments may be implemented using processor 110 without additional instructions stored in memory 115. Some embodiments are implemented using the instructions stored in memory 115 for execution by one or more microprocessors. Thus, the disclosure is not limited to a specific configuration of hardware and/or software.

It is to be understood that the example system shown in the figure is not intended to be limited to the components that may be employed in a given implementation. For example, the system may include one or more additional processors. Furthermore, one or more components of control and processing hardware 100 may be provided as an external component that is interfaced to a processing device. Furthermore, although the bus 105 is depicted as a single connection between all of the components, it will be appreciated that the bus 105 may represent one or more circuits, devices or communication channels which link two or more of the components. For example, the bus 105 may include a motherboard. The control and processing hardware 100 may include many more or less components than those shown.

Some aspects of the present disclosure can be embodied, at least in part, in software, which, when executed on a computing system, transforms an otherwise generic computing system into a specialty-purpose computing system that is capable of performing the methods disclosed herein, or variations thereof. That is, the techniques can be carried out in a computer system or other data processing system in response to its processor, such as a microprocessor, executing sequences of instructions contained in a memory, such as ROM, volatile RAM, non-volatile memory, cache, magnetic and optical disks, or a remote storage device. Further, the instructions can be downloaded into a computing device over a data network in a form of compiled and linked version. Alternatively, the logic to perform the processes as discussed above could be implemented in additional computer and/or machine-readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices. As used herein, the phrases “computer readable material” and “computer readable storage medium” refers to all computer-readable media, except for a transitory propagating signal per se.

The example system shown in FIG. 1B may be employed by an operator to form an incision in skin. As noted above, the formation of an incision in various tissue types (including human skin) via PIRL laser pulses will typically require multiple passes of a scanned beam of laser pulses. Accordingly, in order to form a skin incision with the laser pulse delivery tool 200 of the example system shown in FIG. 1B, it will be necessary to translate the laser pulse delivery tool over multiple passes relative to the skin surface 10.

This requirement for multiple passes of the laser pulse delivery tool presents numerous challenges for the formation of a narrow incision that is substantially free of visible scar tissue. As explained above, one of these challenges is the need to maintain positional accuracy of the distal tip of the optical waveguide relative to the incision. Indeed, since PIRL pulses are capable for forming incisions with very narrow cross-sectional widths, it can be very challenging to achieve the necessary positional accuracy over multiple passes.

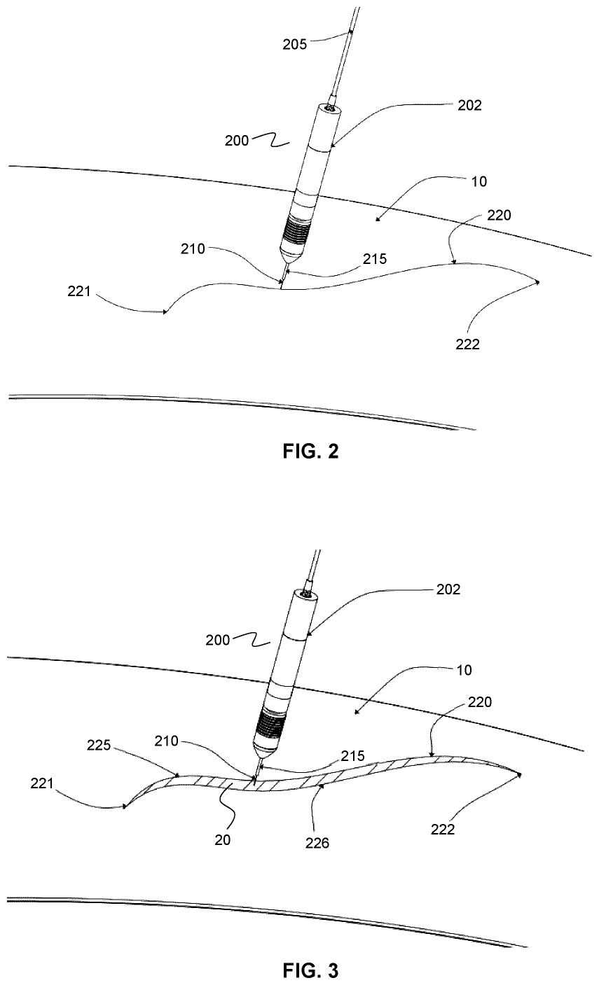

One example embodiment of the present disclosure provides a solution to this problem by providing positional feedback for positioning the laser pulse delivery tool as the laser pulse delivery tool is translated over multiple passes while forming the incision. For example, as shown in FIG. 2, an incision guideline 220 may be defined on the skin surface 10 before initiating the incision. The example guideline 220 is shown extending from a first guideline end 221 to a second guideline end 222. During operation, the operator translates the laser pulse delivery tool 200 over the skin surface, contacting the distal tip of the optical waveguide 210 within the guideline 220.

The guideline 220 may be a physical marking formed on the skin surface 220, such as a contour drawn on the skin surface using an ink marker. Alternatively, the guideline 220 may be an ablative guideline formed via the first pass (or dynamically determined according based on one or more previous passes) of the laser pulse delivery tool 200 relative to the skin surface 10. In such as case the incision guide may be defined by the two edges 225, 226 of the incision 20, as shown in FIG. 3.

In some example implementations, an alignment sensor may be employed to detect a signal indicative of the alignment between the distal tip of the optical waveguide 210 with the incision. The alignment sensor may be located on the laser pulse delivery tool 200, such as sensor 260 shown in FIG. 1B, or may reside remote from the laser pulse delivery tool, as shown by the location of the example alignment sensor 165 in FIG. 1B.

In some example embodiments, an alignment sensor 260 that is secured to the laser pulse delivery tool 200 may be a non-imaging sensor. For example, an inertial sensor may be employed to infer when the distal tip of the optical waveguide 210 has shifted laterally beyond the lateral spatial extent of an incision guide. In another example embodiment, the laser pulse delivery tool 200 may support a light source and an optical sensor that is configured to detect the intensity of the reflection of light (generated by the light source) from a skin region proximal to the distal tip of the optical waveguide 220. A change in the intensity of the reflected light from regions beyond the incision guide (e.g. beyond a marked incision guide or beyond incision edges) may be employed to detect a state of misalignment.

In another example embodiment, an alignment camera having a narrow field of view that is aligned with the distal tip of the optical waveguide 210 may be supported by the laser pulse delivery tool 200. Images recorded by the alignment camera may be processed to determine when the distal tip of the optical waveguide has moved beyond the spatial extent of the incision guide 220 (e.g. beyond a marked incision guide or beyond incision edges), and feedback can be provided to the operator to indicate the state of misalignment.

In other example embodiments, sensors may also be secured to the laser pulse delivery tool 200 to provide feedback information on incision alignment status. Non-limiting examples of sensors include a laser positioning sensor, an Optical Coherence Tomography (OCT) sensor (optical fiber and OCT system), Bragg grating fiber strain sensors, and a piezoelectric sensor, such as a piezoelectric proximity, pressure or temperature sensor.

In some example embodiments, the sensor may be an imaging sensor (e.g. a camera) that is positioned remotely from the laser pulse delivery tool 200 such that the incision guideline 220 resides within the field of view of the camera. Images from the imaging sensor may be recorded and processed during translation of the laser pulse delivery tool 200 and real-time feedback may be provided to the operator for correcting positioning errors. The images recorded by the imaging sensor may be processed, for example, using an object localization algorithm, to detect a location of the incision guideline 220 and a location of the distal tip of the optical waveguide 210 (or a location of another portion of the laser pulse delivery tool 200, from which the location of the distal tip can be inferred). The images recorded by the camera may be processed to identify the incision guide. For example, the incision guide may be intraoperatively determined by processing signals from an imaging camera to determine a real-time spatial extent of the incision, defining the prescribed incision path by a central region of the incision, and determining a state of misalignment when the distal tip of the optical waveguide is determined to reside beyond the central region. Non-limiting examples of suitable image processing algorithms include convolutional neural network algorithms adapted for localization (e.g. region-based convolutional neural networks) and classifier/localization algorithms (e.g. a Harr cascade classification algorithm).

In other example implementations, the laser pulse delivery tool 200 may include trackable fiducial markers that provide position location with a tracking system. For example, the laser pulse delivery tool 200 may include at least three trackable passive or active fiducial markers that are detectable with an optical tracking system having a pair of tracking cameras, where images obtained from the tracking cameras can be processed to determine a real-time position and orientation of the laser pulse delivery tool 200. In such a case, the images from the tracking camera may also be processed to determine the real-time location of the incision. Positional (and optionally orientation) feedback for maintaining the position (and optionally orientation) of the laser pulse delivery tool relative to the incision may be generated based on the real-time knowledge of the position and orientation of the laser pulse delivery tool 200 and the incision guide 220.

In some examples implementations, a surface detection modality, such as, but not limited to, laser radar, structured light, and stereographic imaging, may be employed to detect surface data characterizing a surface profile of the incision. The incision surface data may be processed (e.g. segmented) to detect one or more incision features, such as, for example, the incision trough (bottom), the incision edges, and the incision width. The surface data and/or one or more features computed therefrom may be processed to determine the misalignment of the laser pulse delivery tool relative to the incision and/or to provide feedback for correct alignment of the laser pulse delivery tool relative to the incision.

In some example implementations, the position of an incision guide 220 may initially be determined and represented in a common coordinate system with the tracked position and/or orientation of the laser pulse delivery tool 200. After one or more passes of the laser pulse delivery tool, the incision edges 225 and 226 will separate due to skin tension (and optionally applied tension) and it may no longer be possible to track the initial incision guideline 220. The positions of the incision edges 225 and 226 may then be identified and tracked, with a virtual incision line being determined based on the tracked incision edges. For example, the incision trough (a contour representing the bottom vertex of an elongate incision) may be assumed to reside centrally between the two incision edges 225 and 226. The position of incision line may thus be intraoperatively estimated and employed to detect misalignments of the laser pulse delivery tool 200, and to optionally control the laser source to prevent the delivery of laser pulses to the laser pulse delivery tool (i.e. to the optical waveguide) when the distal tip of optical waveguide is deemed to be misaligned.

As explained in further detail below, in some example embodiments, the tracked separation between the incision edges (or the tracked separation of artificial fiducial markers or inherent anatomical fiducial features on the skin or within the incision) may be employed to infer an estimate of the tension applied across the incision (or within the trough of the incision) and employed to prevent the application of tension that exceeds the elastic deformation of tissue within the incision.

Non-limiting examples of different forms of feedback generated in response to detected misalignment include audible warnings, warnings displayed on a display device, and haptic feedback provided via a haptic actuator (e.g. a piezoelectric device) residing on the laser pulse delivery tool 200. The feedback that is provided when a state of misalignment is detected may optionally further include instruction for how to correct the misalignment.

The feedback may, in some cases, be provided when the distal tip of the optical waveguide 210 resides beyond a prescribed spatial offset relative to a midpoint of the incision guide 220, such as a midpoint of a pre-operatively marked incision guide, or a midpoint that is intraoperatively determined that resides between incision edges. The prescribed spatial offset may be, for example, equal to one half of the width of the beam of laser pulses on the skin tissue when the distal tip of the optical waveguide is contacted with the skin tissue.

In other example implementations, the feedback can additionally or alternatively be implemented as control signals that are sent to the laser system to interrupt the delivery of laser pulses when a state of misalignment is detected. Such an implementation can be effective in preventing the ablation of tissue beyond the region identified by the incision guide, thereby maintaining a narrow incision even when misalignments occur.

It will be understood that many different types of optical waveguides and configurations of optical waveguides may be employed to deliver the laser pulses to the skin surface. In the case of PIRL ablation using laser pulses with a wavelength in the mid-infrared, such as, for example, 2.7-3.3 μm, suitable materials for the optical waveguide (and for the optical fiber delivering the laser pulses from the laser source to the laser pulse delivery tool) include, but are not limited to, sapphire, Y₃Al₅O (YAG), GeO₂ (Germanium Oxid), TeO₂ (Telluriumoxid), ZrF₄, InF₃, AlF₃, endcapped photonic crystal fiber, endcapped hollow core fibers, or other infrared waveguiding fibers.

In some example embodiments, the distal portion of the laser pulse delivery tool that includes the optical waveguide may be removable (e.g. interchangeable). For example, in one example implementation, a plurality of different distal portions may be available and removably attachable to the laser pulse delivery tool, where each different distal portion has a different optical waveguide configuration (e.g. providing a different beam delivery arrangement). The different optical waveguide configurations may include, for example, differently shaped distal tips, different cross-sectional waveguide shapes (e.g. cylindrical fibers and planar waveguides), and the inclusion of one or more additional optical components to facilitate free-space optical coupling between a distal end of the optical waveguide and the tissue through at least one free-space region.

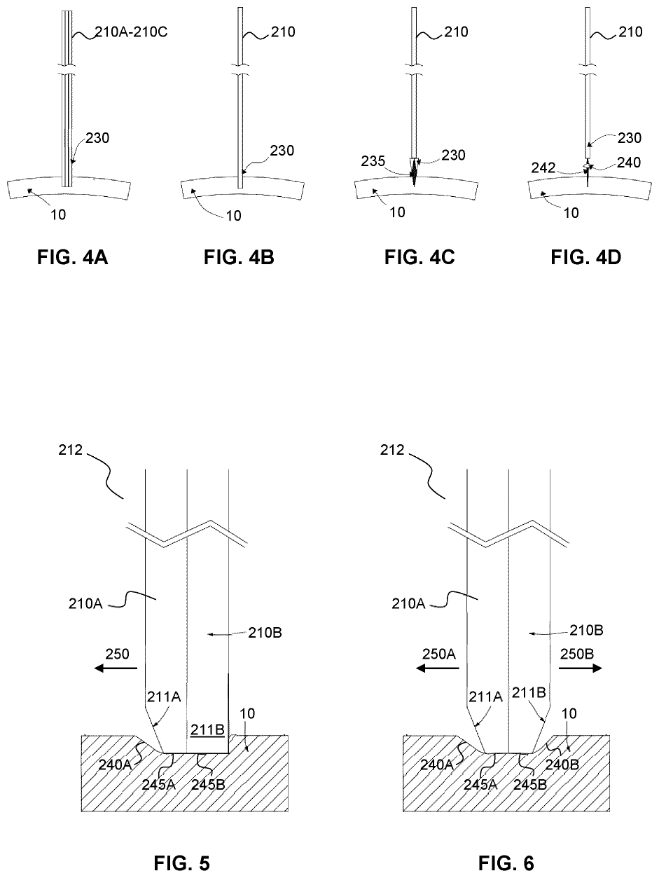

FIG. 4A illustrates an example implementation in which an array of optical waveguides (e.g. optical fibers) 210A-210C are employed for delivering the laser pulses to the skin surface 10. Each of the optical waveguides 210A-210C may be connectable to the optical source such that pulses are simultaneously delivered to each fiber, with the result that an elongate tissue volume residing beneath each of the optical waveguides of the array is ablated in a single ablation event via the parallel delivery of the laser pulses with the distal tip 230 of the array contacting the skin surface 10. Alternatively, the laser pulses may be delivered to the different optical waveguides of the array in a serial, temporally-delayed manner, such that per-waveguide ablation events occur serially. Such an approach may be preferable in order to reduce the amount of residual heat deposition and reduce the likelihood of thermally-induced tissue necrosis and scar tissue formation. In some example embodiments, the laser pulses are delivered serially such that a time delay between laser pulses delivered to adjacent optical waveguides of the array exceeds the thermal diffusion time associated with the diffusion of heat between adjacent residual tissue volumes residing below adjacent optical waveguides of the array.

FIG. 4B illustrates an alternative example implementation in which the optical waveguide 210 is a single optical fiber having a conventional cylindrical distal tip 230.

In some example embodiments, the distal tip of the optical waveguide (or the distal tip of one or more optical waveguides of an optical waveguide array) may be shaped. FIG. 4C illustrates an example embodiment in which a single optical waveguide 210 having a shaped distal tip 230 is employed to deliver the laser pulses to the skin surface 10. As shown in the figure, the shaped distal tip 230 generates a shaped beam 235 that directs the laser beam onto the skin surface 10. Non-limiting examples of shapes of the distal tip include elliptically-shaped distal tip regions and non-symmetric conical distal tip regions.

While many of the example embodiments of the present disclosure involve the contact of the distal tip of the optical waveguide with the skin surface during ablation, it will be understood that in other example implementations, such as those described below in which a guide structure is employed to guide the multi-pass translation of the laser pulse delivery tool, or in which a robotic system is employed to translate the laser pulse delivery tool relative to the skin surface, free-space coupling may be employed to deliver the optical pulses to the skin surface 10. In such a case, one or more optical components may reside between the distal end of the optical waveguide and the skin layer 10 (e.g. for projection, focusing, and/or scanning of the laser beam). An example of such an embodiment is illustrated in FIG. 4D in which a lens 240 is employed to focus the laser beam 242 onto the skin surface 10.

In order to form an incision in human skin according to present example embodiments involving the multi-pass scanning of a beam of PIRL laser pulses relative to the skin surface, it may be necessary to increase the depth of the focal region of the PIRL laser pulses relative to the skin surface during the multi-pass scanning. For example, in cases in which the depth of focus (e.g. as determined by the Rayleigh range) of the laser beam is smaller than the thickness of skin tissue that is ablated to form the incision, the depth of the focal region (e.g. the location of the focus of the laser beam along a direction perpendicular to the skin surface) can be increased while forming the incision in order to maintain the incision trough (the bottom of the incision) within the depth of focus of the laser beam. Such depth advancements may be discrete or continuous. For example, the depth may be advanced on a continuous basis, on a per-pass or multi-pass basis (e.g. once every n complete passes across the full length of the incision, where n represents a whole number greater than zero), according to criteria, such as according to a depth signal that is measured by a sensor generating a signal dependent on the depth of the incision relative to the skin surface, or according to other suitable methods.

In the example case of the use of a laser pulse delivery tool that is manually translated by an operator to form the incision, in the absence of any rigid incision guide structure, the depth may be manually advanced by the operator. This manual advancement of the depth of the incision may be performed based on feedback provided by a depth sensor residing on the laser pulse delivery tool, such as sensor 260 shown in FIG. 1B. In some example embodiments, the signal from a depth sensor can be compared, by the control and processing circuitry 100, to a threshold value to determine when the incision has proceeded to a pre-selected depth value. The control and processing circuitry 100 may then provide feedback to the operator indicating that the desired depth has been reached, and/or the control and processing circuitry 100 can send a signal to the laser source for arresting the delivery of laser pulses to the optical waveguide.

In some example embodiments, the laser pulse delivery tool may include a depth control (extension) mechanism that facilitates an increase in the spatial offset of the distal tip of the optical waveguide relative to the main body of the laser pulse delivery tool. For example, a motor assembly may be incorporated into the laser pulse delivery tool enable extrusion and retraction of the distal tip of the optical waveguide to control the advance of cutting. In another example, detectable markings, such as, but not limited to, electrically conductive, magnetic, or optically reflective or absorbing markers (e.g. in the form of a coating) may be incorporated on a surface that moves with the optical waveguide (e.g. a surface of a protective sleeve or cladding of the waveguide) to facilitate the detection of the location of the optical waveguide relative to the main body, and the generation of feedback or the use of a feedback mechanism to control the position of the optical waveguide. In another example, a lead screw with incrementation steps may be incorporated into the laser pulse delivery tool to facilitate axial motion of fiber tip (i.e. motion parallel to a beam delivery axis of the optical waveguide).

In some example implementations, the depth control mechanism may be actuated in a closed-loop manner according to feedback from the depth sensor such that the depth of the distal tip of the waveguide is advanced to maintain or re-establish contact with the tissue within the trough of the incision. In other example implementations, the depth control mechanism may be actuated in an open-loop manner as described above. As described in further detail below, in some example implementations involving the use of a guiding structure to guide translation of the laser pulse delivery tool during formation of the incision, the guiding structure may form a reference for determining changes in the depth of the incision as determined based on signals from a depth sensor. In other example implementations involving the use of a guiding structure to guide translation of the laser pulse delivery tool during formation of the incision, the guiding structure may include a feature that mechanically actuates the depth advancement mechanism.

In some example embodiments, the laser pulse delivery tool may include an optical waveguide having a distal tip that is shaped such that when the distal tip contacts the skin tissue at a contact location, laser pulses emerging from the distal tip are directed at an ablation location that resides laterally adjacent to the contact location. The ablation location thus resides laterally relative to the distal tip, along a direction that is referred to herein as an ablation direction. Accordingly, when the laser pulse delivery tool is translated in the ablation direction during formation of the incision, skin tissue residing in front of the distal tip is ablated prior to encountering the distal tip. The translation of the laser pulse delivery tool along the ablation direction during formation of the incision may be achieved, for example, via the use of a guide structure that enforces a given orientation of the laser pulse delivery tool during translation and incision formation.

In another example implementation, the translation of the laser pulse delivery tool along the ablation direction during formation of the incision may be achieved by detecting, with an orientation sensor, the orientation of the laser pulse delivery tool during incision formation, and providing feedback to the operator to maintain proper alignment of the laser pulse delivery tool during translation. Such an example embodiment may be useful in preventing or reducing the application of shear forces to the tissue within the incision as the laser pulse delivery tool is translated during formation of the incision.

FIG. 5 illustrates an example embodiment, in which a fiber array is provided having an outer optical fiber 210A with a distal tip configured to direct the emitted laser pulses at an ablation location 240A that resides laterally adjacent to a contact location 245A at which the outer optical fiber 210A contacts the skin tissue 10, such that when the optical fiber array is translated along an ablation direction 250, the ablation of skin tissue in front of the outer optical fiber 210A “clears the path” ahead for the translation of the optical fiber array, as described above. One or more optical fibers of the fiber array, such as optical fiber 210B shown in FIG. 5, may have a respective distal tip 211B that is configured to direct the emitted laser pulses axially toward its respective contact location 245B. Accordingly, while the outer optical fiber 210A “clears the path” in front of the optical fiber array, one or more additional optical fibers of the fiber array may be employed to perform ablation of the underlying tissue. In some example implementations, the repetition rate of laser pulses delivered to the outer optical fiber 210A (having the distal tip contoured for lateral emission of laser pulses) may be dependent on the speed of translation of the fiber array (which may be determined, for example, via a sensor supported by the laser pulse delivery tool, such as an inertial sensor or an imaging sensor). In example embodiments involving a guiding structure, the laser pulse delivery tool and the guiding structure may be provided with a keyed mating configuration that enforces the alignment of the optical fiber array along the incision path prescribed by the guiding structure.

FIG. 6 illustrates an example embodiment in which both outer optical fibers 210A and 210B of an optical fiber array 212 have respective distal tip regions 211A and 211B configured to direct the emitted laser pulses at respective ablation locations 240A and 240B that reside laterally adjacent to the contact locations 245A and 245B at which each outer optical fiber contacts the skin tissue. Although the example embodiment shown in FIG. 6 only shows a dual-fiber array, it will be understood that one or more intermediate fibers may also be included between the two outer optical fibers 210A and 210B. The configuration shown in FIG. 6 enables the bi-directional ablative clearing of tissue in front of and behind the fiber array, along directions 250A and 250B. In one example implementation, laser pulses may be delivered through both outer optical fibers, irrespective of the translational direction of the fiber array. In another example embodiment, laser pulses may be selectively delivered to the forward-facing fiber and not the rear-facing fiber so that the laterally-directed pulses are only delivered in front of the fiber array to clear remove tissue from the forward path of the fiber array. The selection of the appropriate outer fiber for delivery of laser pulses may be determined, for example, via signals obtained from a sensor capable of sensing direction, such as an inertial sensor.

In the case of implementations that involve the contact of the distal tip of an optical waveguide (e.g. an optical fiber) with the skin tissue during formation of the incision, it can be difficult to maintain a suitable depth of the distal tip of the optical waveguide relative to the incision that maintains contact of the distal tip of the optical waveguide with the skin tissue while avoiding the application of strain that exceeds the elastic deformation limit of the underlying tissue. In some cases, an excessive amount strain, such as an amount that can cause inelastic tissue deformation and associated scar tissue formation, can occur when the depth of the distal tip of the optical waveguide is advanced too quickly relative to the laser ablation rate during formation of the incision.

While this may be the case for any contact-based implementation, it is especially the case when employing a laser pulse delivery tool to form an incision without the presence of a guiding structure or robotic control. The present inventors found that the occurrence or likelihood of such high-strain events during incision formation can be reduced or prevented by employing a sensor to detect a signal associated with the application of force to the distal tip of the optical waveguide and employing the signal to generate feedback suitable to avoid or reduce the continued application of strain by the distal tip of the optical waveguide.

In some example implementations, a force (pressure) sensor may be integrated with the laser pulse delivery tool for sensing the application of force to the distal tip of the optical waveguide. Non-limiting examples of suitable sensors include, an optical sensor located for detecting back reflection feedback of the optical waveguide, a piezo pressure sensor incorporated in the laser pulse delivery tool, a Bragg fiber grating strain sensors integrated into the fibre delivery system, and an optical deflection sensor (explained below).

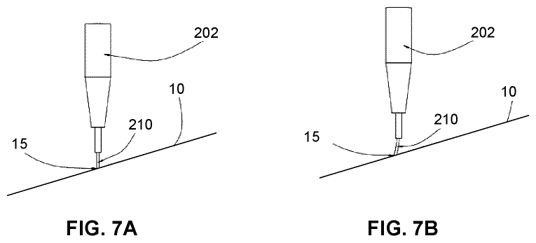

In some example embodiments in which the optical waveguide is deflectable, such as in the case of an optical fiber, the force sensor may be a deflection sensor. FIGS. 7A and 7B illustrate the deflection of the optical waveguide 210 via the application of excessive force to the underlying tissue 10 at a contact point 15. In FIG. 7A, the force applied to the distal tip of the optical waveguide is small and the distal tip is not substantially deflected and thus lies along the elongate axis of the optical waveguide. In FIG. 7B, a force applied to the distal tip of the optical waveguide 210 causes deflection of the distal tip. Non-limiting examples of the deflection sensor include strain gauges, imaging sensors capable of detecting changes in the location of the distal tip, reflectometric sensors involving the detection of an optical beam reflected from a reference location, a soft fiber sleeve with an embedded conductive sensor coating, and hall effect sensors, integrated fiber Bragg grating strain sensors.

Feedback may be generated that is dependent on the signal detected by the force sensor. For example, feedback may be generated when the signal exceeds a pre-established threshold. This threshold may be determined, for example, through numerical simulations of the mechanical response of tissue to the application of force by the distal end of the waveguide, and/or based on experiments that relate the force sensor signal to the tissue elongation with the force adjusted to keep the tissue from undergoing strain changes exceeding the elastic limit, typically taken to be 1% strain. The specific tissue dependent limit can be calibrated on model tissue with respect to onset of scar tissue. In some implementations, the threshold may be provided to accommodate a safety margin in order to reduce the likelihood of tissue damage.

Non-limiting examples of different forms of feedback include audible warnings, warnings displayed on a display device, and haptic feedback provided via a haptic actuator (e.g. a piezoelectric device) residing on the laser pulse delivery tool 200.

In other example implementations, the feedback can additionally or alternatively be implemented as control signals that are sent to the laser system to modify the delivery of laser pulses according to the signal detected by the force sensor. For example, the rate of delivery of laser pulses to the optical waveguide may be increased when an increase in force is detected, such a rate of ablation of tissue is increased, thereby removing tissue that would otherwise experience inelastic deformation in the presence of contact by the distal end of the optical waveguide, and thereby reducing the detected force. Such an implementation can be effective in preventing or reducing the application of strain beyond the elastic deformation limit.

In other example implementations, the feedback can additionally or alternatively be employed as control signals that are sent to the laser pulse delivery tool to control a depth advancement mechanism residing on the laser pulse delivery tool. For example, when the force sensor detects a signal indicative of an excessive amount of applied force (e.g. when the force-dependent signal exceeds a pre-determined threshold), control signals may be transmitted to the depth advancement mechanism that cause the depth advancement mechanism to reduce the depth of the distal end of the optical waveguide relative to the skin surface (or, for example, a reference surface associated with a guiding structure).

As noted above, in cases in which the translation of the laser pulse delivery tool is manually controlled during the formation of an incision via the delivery of PIRL laser pulses over multiple passes, it can be difficult to form a uniform incision due to an inability to control the temporal and spatial delivery of the laser pulses over the length of the incision. Indeed, when the laser pulse delivery tool is employed to deliver PIRL laser pulses to the skin, with the laser pulse delivery tool being moved by an operator along the incision path, variations in the rate of translation of the laser pulse delivery tool can lead to the local accumulation of residual heat along incision path. Such local hotspots along the incision path can undergo tissue necrosis and scar tissue formation if the accumulated heat exceeds a threshold. Furthermore, when the laser pulse delivery tool is not uniformly translated along the incision path, such variations in the rate of the translation can also generate local variations in the depth of the incision along the incision path due to different dwell times of the tool along the incision path.

The present inventors found that this problem can be addressed by controlling the rate of delivery of laser pulses to the optical waveguide, such that the rate of delivery of laser pulses is dependent on the rate of translation of the laser pulse delivery tool. By controlling the rate of delivery of laser pulses in response to the speed of translation of the laser pulse delivery tool, the residual heat deposited to the underlying tissue within the incision can uniformly distributed along the incision path. Moreover, a uniform incision depth along the incision path can be achieved.

A number of different sensor geometries and configurations can be employed to detect a signal dependent on the speed of the laser pulse delivery tool. In some example implementations, the sensor may be supported by, and co-moving with, the laser pulse delivery tool, as illustrated by sensor 260 in FIG. 1B. Example configurations in which the speed-sensing sensor is supported by the laser pulse delivery tool include, but are not limited to, inertial sensors that sense changes in speed and imaging sensors that generate images which can be processed to infer speed.

In example implementations in which translation of the laser pulse delivery tool is guided by a guiding structure that is secured to the skin surface of the patient, the guiding structure may include a plurality of reference markers that facilitate the determination of speed via a passive or active sensor residing on the laser pulse delivery tool. For example, the laser pulse delivery tool may include a light source and a photodetector, where the photodetector detects changes in reflectivity that are modulated by markings on the guiding structure, where the markings are provided with a known spatial separation. In another example, the laser pulse delivery tool may electrically contact the guiding structure during translation and the guiding structure may exhibit a spatially-varying resistivity along its length, thereby facilitating the detection of an electrical signal indicative of the speed of the laser pulse delivery tool relative to the guiding structure. In another example, the laser pulse delivery tool may include a magnetic field sensor that detects the presence of a plurality of magnets that are provided along the length of the guiding structure.

Alternatively, the sensor may reside external to the laser pulse delivery tool, as shown by sensor 165 in FIG. 1B. For example, the external sensor may be an imaging sensor that is capable of generating images that include the laser pulse delivery tool and which can be processed (e.g. via methods described above) to determine changes in the speed of translation of the laser pulse delivery tool.

In example embodiments in which translation of the laser pulse delivery tool is guided by the presence of a guiding structure secured to the skin surface of the patient, the guiding structure may include one or more sensors that are capable of generating signals (e.g. changes in magnetic field, changes in optical reflectivity, or changes in electrical resistance) as the laser pulse delivery tool moves relative to the guiding structure.

In some example implementations, the laser source can be controlled to deliver the laser pulses according to a pre-determined relationship between the speed-dependent signal (e.g. according to a measure obtained by processing the speed-dependent signal) and a pulse repetition rate. This relationship may be prescribed such that a spatially uniform deposition of laser energy is provided along the incision path in the absence of thermally-induced scarring. In some example implementations, the laser source can be controlled to such that the pulse repetition rate is prevented from exceeding a speed-dependent pulse repetition rate threshold. The relationship and/or speed-dependent pulse repetition rate threshold may be determined, for example, through numerical simulations of the deposition of residual heat during PIRL ablation, and/or based on experiments that determine the dependence of pulse repetition rate and laser pulse delivery tool translation speed on the degree of formation of scar tissue.

In other example embodiments, the rate of delivery of laser pulses can be controlled according to real-time measurements of signals dependent on the local temperature of the residual tissue within the incision. By controlling the rate of delivery of laser pulses in response to the locally-determined temperature of the residual tissue (tissue remaining after ablation), the residual heat deposited to the underlying tissue within the incision can limited to avoid tissue necrosis. Moreover, the delivery of laser pulses to may be controlled to achieve a uniform residual temperature profile along the incision path.

A number of different sensor geometries and configurations can be employed to detect a signal dependent on the local temperature of the residual tissue within the incision. In some example implementations, the sensor may be supported by, and co-moving with, the laser pulse delivery tool, as illustrated by sensor 260 in FIG. 1B. Example configurations in which the thermal sensor is supported by the laser pulse delivery tool include, but are not limited to inferred thermal imaging cameras, radiation thermometers, optical pyrometers, resistance temperature detectors, and ultrasonic sensors. In the case of thermal cameras, a closely spaced sensor with IR optics provides a thermal image. Optical pyrometers use similar optical arrangements with temperature dependent probes in contact with the skin and selective wavelength monitoring of the emission from the sensor. Ultrasonic sensors must be in direct contact with the tissue for imaging of the sound field generated by the laser ablation process. Thermal sensors of any kind are placed in direct contact with the tissue, with a minimum spatial separation from the position to be excised

In some example implementations, the temperature sensor is provided on the laser pulse delivery tool such that the temperature is measured in a forward direction associated with the direction of translation of the laser pulse delivery tool. Such a configuration enables the determination of a local temperature within an adjacent local tissue region that is adjacent to the currently laser-irradiated region and which will be subsequently ablated during translation of the laser pulse delivery tool and delivery of the laser pulses. Such a configuration enables the control of the laser pulse delivery rate (and the resulting deposition of residual heat) to the adjacent local tissue region based on the locally-sensed temperature-dependent signal.

In some example implementations, a dual sensor configuration may be employed in which thermal sensors are provided in both directions, relative to the laser pulse delivery tool, along the incision path (i.e. along the ablation direction). Such a configuration enables the bi-directional sensing of two temperature-dependent signals. The direction of travel of the laser pulse delivery tool may be detected (e.g. via an inertial sensor or other suitable sensor) and employed to determine which of the two temperature dependent signals currently corresponds to a forward-looking direction and relates to the interrogation of an adjacent local tissue region that will be ablated during subsequent translation of the laser pulse delivery tool along the current translation direction. In example embodiments involving a guiding structure, the laser pulse delivery tool and the guiding structure may be provided with a keyed mating configuration that enforces the alignment of the thermal sensors along the incision path prescribed by the guiding structure.

In other example implementations, a sensor may reside external to the laser pulse delivery tool, as shown by sensor 165 in FIG. 1B. For example, the external sensor may be optical imaging thermo/pressure/deflection sensors, and ultrasonic sensors. In example embodiments in which translation of the laser pulse delivery tool is guided by the presence of a guiding structure secured to the skin surface of the patient, the guiding structure may include a plurality of thermal sensors.

In some example implementations, the laser source can be controlled to deliver the laser pulses according to a pre-determined relationship between the temperature-dependent signal (e.g. according to a measure obtained by processing the temperature-dependent signal) and a pulse repetition rate. This relationship may be prescribed such that a spatially uniform deposition of laser energy is provided along the incision path in the absence of thermally-induced scarring. In some example implementations, the laser source can be controlled to such that the pulse repetition rate is reduced when the signal associated with the local temperature exceeds a threshold. The relationship and/or threshold may be determined, for example, through numerical simulations of the deposition of residual heat during PIRL ablation, and/or based on experiments that determine the dependence of local temperature on the degree of formation of scar tissue.

While some of the preceding example embodiments refer to the use of sensors for sending a force (or pressure) applied to the optical waveguide, or, for example, to detect the speed of translation of the laser pulse delivery tool, or, for example, to detect signals associated with the local temperature of tissue regions along the incision path, it will be understood that other types of sensors (e.g. employing other sensing modalities), and/or other sensed physical parameters, may be additionally or alternatively be employed.

In some example implementations, sensors such as optical spectroscopic sensors or mass spectroscopic sensors may be used to detect signals correlated to tissue damage. These may include thermally denatured biomolecules detected in the laser plume by specific characteristic masses using mass spectrometry or optical detection of thermally denatured biomolecules as coagulation and carbonization processes. These detector outputs may be processed to determine a presence of tissue damage, and subsequently employed to provide feedback including, but not limited to, warnings provided to an operator, and control signals delivered to the laser source or laser pulse delivery tool to prevent further tissue damage. For example, optical spectroscopic sensing (e.g. employing Raman detection) may be employed to detect tissue necrosis and/or blood vessels.

In some example embodiments, a structure may be secured to the surface of the skin of the subject to assist with the formation of the incision in a controlled manner. For example, a tensioning structure may be provided that facilitates the application of tension during the formation of the incision. In other example implementations, the translation of the laser pulse delivery tool (laser pulse delivery tool) for the formation of the incision via multiple passes may be guided (constrained) by a guide structure that is secured to the skin surface. For example, an incision guide structure may be provided that includes an elongate aperture that is configured to receive and guide translation of a laser pulse delivery tool. In some example embodiments, a guide structure may be provided that is configured both for guiding translation of a laser pulse delivery tool and for applying a controlled amount of tension during formation of the incision, as described in further detail below.

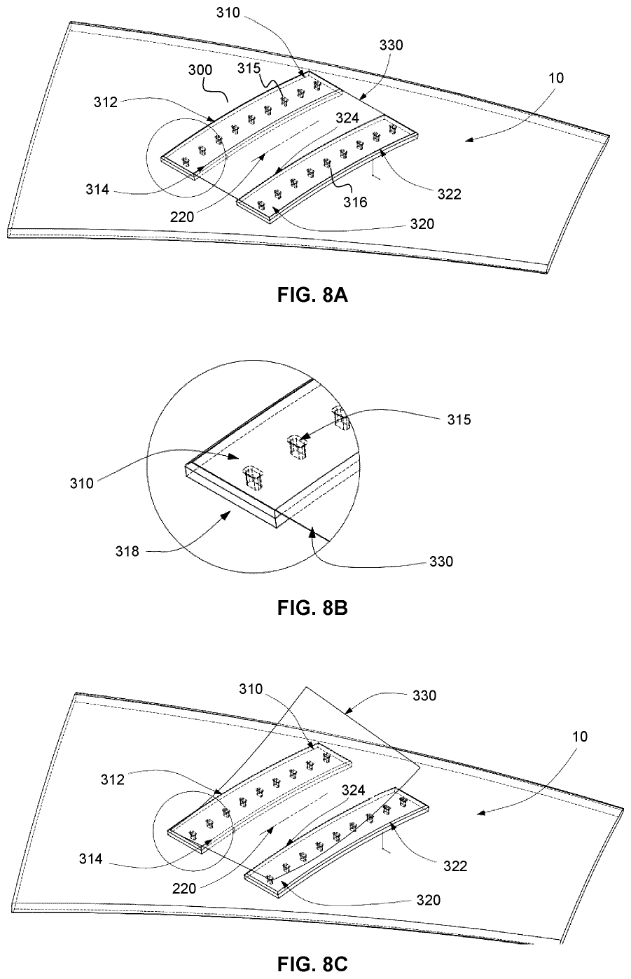

In some example implementations, a support substrate (base) is secured to the patient skin surface, the support substrate being configured to support the guide structure relative to the skin of the patient. FIGS. 8A-8C illustrate an example of a support substrate 300, which includes a first elongate substrate portion 310 and a second elongate substrate portion 320 having respective outer edges 312 and 322, and respective inner edges 314 and 324, and which are removably attachable to the skin surface 10. As shown in the figure, the first elongate substrate portion 310 and the second elongate substrate portion 320 are provided in a spaced relationship, such that the skin surface 10 is accessible between the first elongate substrate 310 portion and the second elongate substrate portion 320.

The first and second elongate substrate portions 310, 320 may be flexible and capable of conforming to the curvature of the underlying skin surface 10. Non-limiting examples of suitable materials for forming the first and second elongate substrate portions include soft composites of fiber and silicone, nitinol sheets, fast-casting silicon, polyamide, and other flexible materials. The underside of each elongate substrate portion (e.g. the underside 318 of first elongate substrate portion 310 shown in FIG. 8B) may be provided with an adhesive material suitable for removably adhering the elongate substrate portions to the skin surface. The adhesive material may be an adhesive glue such as surgical glue or cyanoacrylate, or any other adhesive material which is not toxic to the skin. In some implementations, the adhesive may be a material suitable for remaining adhered to the skin for a time duration of one or more days or one or more weeks. Alternatively, a non-adhesive attachment method may be employed, such as vacuum suction applied through one or more ports or channels defined within the support substrate 300.

While FIG. 8A illustrates an example implementation in which the first elongate substrate portion 310 and the second elongate substrate portion 320 are physically separate, in other example implementations, the first and second elongate substrate portions 310, 320 need not be physically distinct and may be physically connected while enclosing a central aperture that permits access to the skin region for formation of the incision with the aperture. For example, a single monolithic support substrate may be provided having a central aperture that is enclosed, in part, by the elongate substrate portions 310 and 320.

Example implementations in which the first elongate substrate portion 310 and the second elongate substrate portion 320 are physically separate may be beneficial in facilitating the application of tension across the incision during formation of the incision. Such a configuration may also be beneficial in facilitating the opening of the incision for the insertion of surgical tools without needing to remove the elongate substrate portions, and the optional use of the elongate substrate portions to support intraoperative incision opening and/or post-operative incision closure devices, as described in detail below.

As shown in FIGS. 8A-8C, an alignment member, such as an alignment film 330 having an adhesive coating, may be initially secured to the first and second elongate substrate portions 310, 320 to facilitate positioning and alignment of the first and second elongate substrate portions on the skin surface with a prescribed spacing. The alignment member 330 may then be removed to permit access to the skin tissue region residing between the first and second elongate substrate portions 310, 320, as shown in FIG. 8C.

The alignment member may be transparent in order to facilitate positioning and alignment of the first and second elongate substrate portions 310, 320, relative to a desired incision location. For example, the first and second elongate substrate portions may be positioned such that they are equidistant from an incision guideline 220 marked on the skin surface. In some example implementations, a marked guideline 220 on the skin surface 10 can facilitate positioning of the first and second elongate substrate portions 310, 320 in the absence of an additional alignment member.

In some example implementations, the alignment member is configured such that an initial spacing of the first and second elongate substrate portions 310, 320 is selectable prior to attachment of the first elongate substrate portion and the second elongate substrate portion to the skin surface. For example, the alignment member may include a variable positioning mechanism. In another example implementation, a set of alignment members 330 may be provided with different spacing widths, for example, ranging from 1 cm to 10 cm, for facilitate the placement of the first and second elongate substates at different spacings. In other example implementations, the alignment member may be extendable (and may be made, for example, of a polymer composite) to permit selection of a desired spatial separation between the first and second elongate substrate portions.

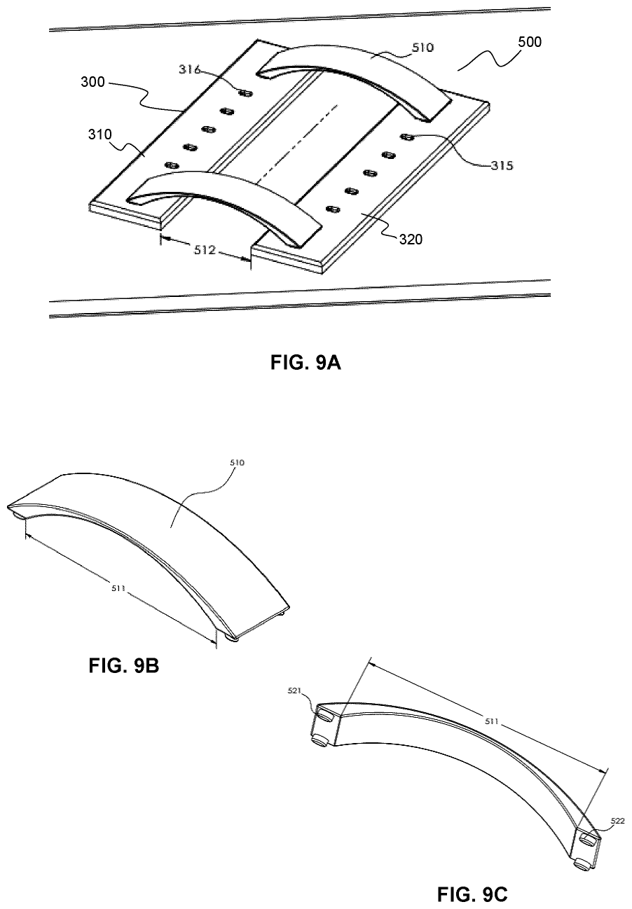

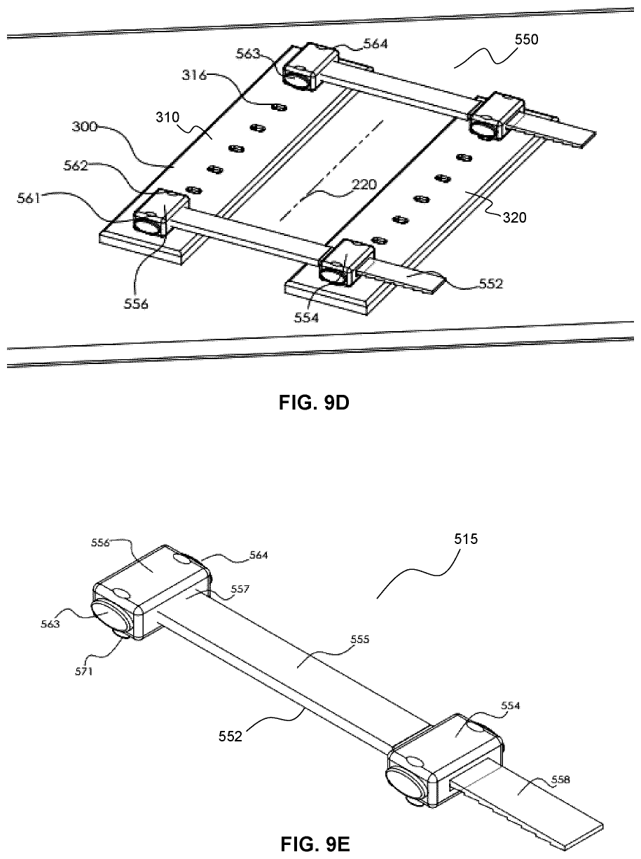

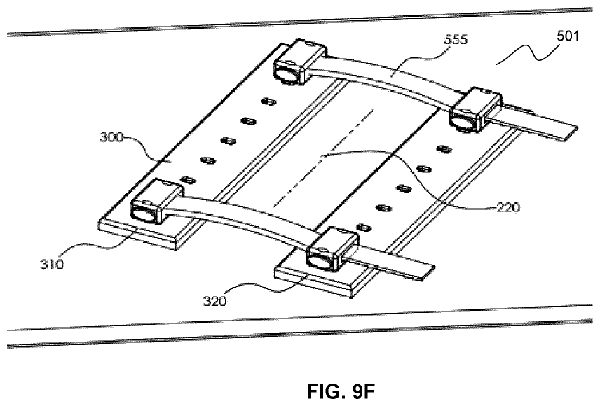

In one example embodiment, the tension control structure 500 is configured as single elastic bridge structure as illustrated in FIGS. 9A-C. As shown in FIG. 9A, the support substrate 300 may be employed to provide a support (base) structure for a tensioning control structure having a tensioning mechanism 510 capable applying tension across the incision during formation of the incision.

The example elastic tensioning structure 510 shown in FIG. 9A is structured as bridge shape where the tension is applied by elastic bridge members 510. Elastic bridge members 510 are secured to the elongate substrate portions 310 and 320. For example, the elastic bridge structure 510 may include engagement features 521 and 522, as shown in FIG. 9C that are configured to engage with corresponding engagement features (e.g. liner slots 315 and 316) in elongate substrate portions 310 and 320.

The elastic bridge structures 510 are secured to elongate substrate portions 310 and 320 while applying a deformation force to the elastic bridge structure 510, the deformation force being applied such that the elastic bridge structures 510 contract (reduce) in length (i.e. in a direction perpendicular to the incision). For example, the elastic bridge members 510 may be pinched while securing the elastic bridge members 510 to the elongate substrate portions 310 and 320. After the elastic bridge structures 510 are mechanically engaged with the elongate substrate portions 310 and 320, the deformation force is withdrawn, and, the distance 512 between the elongate substrate portions 310 and 320 is increased. More specifically, when the elastic bridge structures 510 are fixed in place while applying a compressive force to the elastic bridge structures 510, stress resides in the elastic bridge structures 510. After removal of the compressive force, horizontal stress is then transferred to the engagement features 521 and 522, promoting separation of the attached elongate substrate 310 and 320, which successfully delivers limited tensioning to the incision skin area.

Non-limiting examples for material selection of the elastic bridge 510 includes: nitinol composites, nitinol structured sheet, flexible plastic, rubber, silicon. The tension applied to the incision area is a function of the material elastic modulus and initial distance of the elastic bridge structures 510 and the initial distance between two elongate substrate 310 and 320. The example elastic bridge structures 510 may be provided in a wide range of sizes, for example, to accommodate initial separations of the elongate substrate 310 and 320, for example, over a range of 2-10 cm The applied tension may reduce slightly during incision opening, but may still be sufficient to continue to open the incision, providing a structure that is easy to operate. In this example embodiment, the tension is not controllable during the application and is limited to the bending stress applied by the elastic bridge.

FIGS. 9D-9F illustrate another example of a tension control structure 550, which is configured as linear tension mechanism. This example tension control structure 550 consist of three parts, fixation part 556, tensioning part 554 and tensioning belt 552. Engagement features 571/572 are positioned on the bottom surface of fixation part 556 and tensioning part 554, which are compatible with any of two liner slots 315/316 on elongated substrates 310,320. As shown in the figure, additional engagement features, such as liner slots 561 and 562 may be provided within the upper surface of fixation part 556 and tensioning part 554 in order to facilitate the engagement of additional structures, such as the incision guide structure 350 or incision tension structure 600 described in further detail below. The proximal end 557 of the tensioning belt 55 is fixed to the fixation part 556, for example, by a fixation means such as, but not limited to, glue and joints. The distal end 558 of tensioning belt 552 is configured to have spaced slits/thread. The tensioning part 554 contains mating spaced nuts/slits/teeth which can efficiently grip on tensioning belt 552 without sliding.

To prepare for use, the tensioning control structure 550 is secured onto the elongated substrate portions 310/320 using the engagement features. rigidity The distal portion of the tensioning belt 558 is then extended through the tensioning part 554, resulting in an increase in the spatial separation between the two elongated substrate portions 310/320, which produces tension within the tensioning belt 552 that results in the application of tension across the incision region 220. Non-limiting examples of the material of tensioning belt can be HDPE, PP, rigid silicon. Two release bottoms 563,564 located on the sides of fixation part 556 and/or the tensioning part 554 provide a release mechanism for releasing the tension applied by the tensing belt 552.

In some example implementations, the tensioning belt 552 can be formed from a material that exhibits elasticity and thus deforms during tensioning, as shown in FIG. 9F. As the belt is pushed through the tensioning party 554, compression force is acted on the central portion 555 of the tensioning belt 552. The center portion of the belt will then raise while introducing elastic compression on two elongated substrates, promoting tension force on the incision area. Non-limiting examples of suitable materials for the central portion 555 of the tensioning belt 552 include rubber, silicon, flexible plastic, or nitinol with a stiffness modulus ranging from 0.5-0.7 Gpa. Forming the tensioning belt from an elastic material may be beneficial in that the elasticity enables the device to deform laterally in small portion during operation, acting as a vertical strain relief for uneven skin.

Accordingly, in some example embodiments, the tension control structure may include a tensioning mechanism that can be manually configured (e.g. adjusted, tuned) to applying a desired amount of tension across the incision. For example, the tensioning mechanism may include pulling tabs, rod with positioning teeth, threaded mechanism, suction/pressure mechanism, etc. The application of tension may be beneficial in improving the quality of the incision, provided that the tension is insufficient to cause the tissue within the incision to experience inelastic deformation. In some example implementations, the application of tension to the skin during formation of the incision may be beneficial in defining a planar region on the skin surface that can be employed as a reference plane when controlling the depth of the focused laser pulses when forming the incision.

In some example embodiments, the tension control structure may include a tensioning mechanism that is connectable to an external device to facilitate the automatic and/or remote-controlled application of tension across the incision. For example, the tension control structure may be connectable to a controller that is configured to receive input from an operator for applying a controlled and selectable amount of tension across the incision.

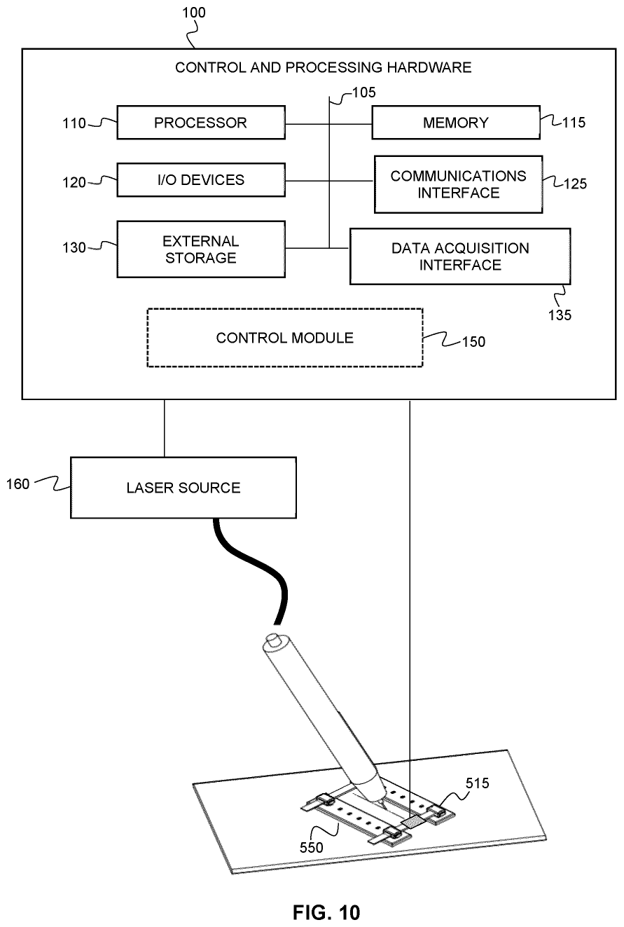

The tension control structure may include a sensor that is capable of generating a signal dependent on the applied tension. Non-limiting examples of sensors include strain gauges and piezoelectric sensors. The signal may be processed by the control and processing hardware 100 to determine whether or not the applied tension is likely or expected to result in the generation of forces within the incision that are below the elastic deformation limit of tissue within the incision. FIG. 10 illustrates an example implementation in which a tension control structure 550 includes a tensioning mechanism and tension sensor 515 that are connected (or are connectable) to the control and processing hardware 100.

Although the tension applied to the skin surface by the tensioning mechanism may be different than the tension applied within the center (bottom) of the incision, a relationship may be established between the signal generated by the sensor and the internal strain that is generated within tissue residing at the center of the incision. The relationship may be determined, for example, via simulations and/or experiments. In the latter case, the strain experienced by the tissue residing at the center (bottom) of the incision may be inferred via the processing of overhead images of the incision, as explained in further detail below. The relationship may be expressed in a continuous or discrete form. For example, the relationship may be defined as a signal threshold beyond which the applied strain within the incision (e.g. at or near the center/bottom/vertex of the incision) is expected to exceed the elastic deformation limit, or is expected to be at risk of exceeding the elastic deformation limit (e.g. is associated with a margin or guardband).

In some example implementations, one or more optical modalities may be employed to determine a measure associated with the strain applied within the incision by the tensioning mechanism. For example, an imaging camera having a field of view that includes the incision edges and the bottom trough of the incision when the incision is sufficiently opened. The imaging camera may be provided at a location that is substantially overhead, relative to the incision, the permit imaging of the center (bottom) of the incision when the incision is sufficiently tensioned. For example, the imaging camera may reside on a laser pulse delivery tool that is translated during formation of the incision or the imaging camera may reside externally from the laser pulse delivery tool.

In some example implementations, a plurality of images of the incision may be acquired that respectively correspond to different amounts of tension applied by the tensioning mechanism. The images may be processed to determine a measure associated with the amount of local strain applied within the incision. The measure associated with local strain may be employed to generate feedback suitable for avoiding the application of a local strain that exceeds the elastic deformation limit of the tissue within the incision.

In one example implementation, images may be acquired by the imaging camera as the tension applied by the tensioning mechanism is varied (e.g. increased) and the acquired images may be processed to determine a measure associated with deformation of the tissue within the incision. For example, image processing and localization methods such as those described above may be employed to locate the incision within a plurality of images. The images may then be processed to determine at least one local measure of tissue deformation (e.g. strain). Such a local deformation measure may be obtained, for example, by performing image registration between a common tissue region within the incision among multiple images and employing a deformation measure during image registration to determine the tissue local strain. In another example implementation, fiducial features may be identified and employed to infer a measure of local strain. For example, features may be identified and localized, for example using a convolutional neural network or PCA reconstruction, and two adjacent features that line along the direction of applied tension may be selected. The change of separation between these features at different amounts of applied tension may be employed to determine an estimate of local strain.

The inferred local strain values associated with the plurality of images, and respective measures of applied tension corresponding to the plurality of images, may then be employed to estimate or infer at least one applied tension that results in elastic deformation of the tissue within the incision. For example, a range of tensions applied by the tensioning mechanism (or, for example, a range of tensioning control signals delivered to the tensioning mechanism) may be identified that result in a linear relationship between applied tension (tensile stress) and inferred local strain. Alternatively, a range of tensions applied by the tensioning mechanism (or, for example, a range of tensioning control signals delivered to the tensioning mechanism) may be identified that result in inferred local strain values that are expected, based on simulations, experiments or reference data, to result in elastic deformation of tissue within the incision.

While the preceding example implementations relate to use of images to infer strain with the incision region, such methods may additionally or alternatively be employed to detect applied strain at the tissue surface (e.g. using deformation-based image registration and/or surface fiducials such as moles and other anatomical fiducial features). A pre-established relationship between surface strain and intra-incision strain may then be employed to infer to local strain applied within the incision.

In some example implementations, the edges of the incision may be optically monitored as a function of applied tension, applied force, or another suitable measure associated with the application of tension. The locations of the tissue edges can be detected and localized, for example, using machine learning algorithms as described above. The application of force or tension to open the incision to permit the advancement of one or more surgical tools may also be monitored in this manner. The applied tension (or a measure associated therewith) may be varied while monitoring the edge displacement as the width of the incision increases. The transition from elastic deformation to inelastic deformation may be determined by detecting a change in slope of the displacement (edge movement) with the applied tension from the linear response (F=−Kx), thereby permitting the identification of a maximum applied tension value (or associated measure) that corresponds to elastic deformation.

Without intending to be limited by theory, it is believed that there will be at least two changes in slope as a function of applied force. A first change may occur as an increase in slope as the tissue freed by the incision is drawn taught to compensate for the removal of tissue in the restoring force of the tissue or elastic response. This point defines an initial linear response displacement of the trough of the removed tissue. A second change in slope or displacement may occur as the tissue at this point in space goes beyond the elastic deformation limit, resulting in a decrease in slope or equivalently, a larger displacement for the same applied force. Once this end point is determined, the applied tension may be reduced to lie within linear limits associated with elastic deformation.

By maintaining the applied tension these limits (initial displacement of tissue at the cut boundary/trough and onset of plastic deformation), the tissue within the incision (at the borders of the incision) will be maintained within the elastic limit. The constituent cells of the tissue remain intact throughout the tissue in the elastic limit, an everyday experience in touching skin, and thereby do not trigger the healing mechanism associated with damaged cells an excessive fibroblast formation that leads to scar tissue formation. Accordingly, the present example methods may provide a means to maintain the tissue within the elastic deformation range through at least a portion of a surgical procedure, or in some example implementations, through the entirety of the surgical procedure to eliminate or minimize scar tissue formation that typically limits the efficacy of surgeries.

The present example implementation employs optical imaging for dynamic real-time incision monitoring, and devices such as piezoelectric transducers, motors, actuators, and or other devices for varying the applied tension. However, it will be understood that any other suitable method of measuring strain (e.g. 3D volume changes or displacement) as a function of applied force may be employed in the alternative. In some example implementations, strain sensors such as, but not limited to, fiber Bragg grating sensors, or piezo elements attached to the skin in the near periphery of the cut zone may also be employed to provide real-time strain measures.

In some example implementations, an image may be processed to infer a width of the incision (for example, using image processing methods described above for incision localization). The inferred width, along with a known depth of the incision (which may be inferred, for example, based on signals from a depth sensor, or based on a known depth status of a depth control mechanism, as described herein), may be employed, according to pre-determined criteria, to determine whether or not the incision is sufficiently narrow to avoid inelastic deformation of the tissue within the incision. For example, the criteria may prescribe a maximum width, as a function of incision depth, that can avoid the application of strain within the incision that would result in inelastic tissue deformation.

If the applied tension is deemed to potentially cause or risk causing the inelastic deformation of tissue within the incision, feedback can be generated to facilitate a reduction of the applied tension. Non-limiting examples of different forms of feedback generated in response to a detection of potential inelastic deformation of tissue within the incision include audible warnings, warnings displayed on a display device, and haptic feedback provided via a haptic actuator (e.g. a piezoelectric device). In other example implementations, the feedback can additionally or alternatively be implemented as control signals that are sent to the tensioning mechanism to decrease the applied tension.

In one example implementation, incision images acquired by an imaging camera may be processed to determine whether or not the tension applied by the tensioning mechanism has sufficiently widened the incision. For example, when forming the deeper portion of the incision, an insufficient amount of tension applied across the incision may result in closure or narrowing of the incision.

In the case of an incision formed by a laser pulse delivery tool having an optical waveguide configured to delivery laser pulses to the tissue when the distal tip of the optical waveguide contacts or is positioned proximal to the tissue, an incision that is too narrow may result in contact between the incision sidewalls and the lateral surface of the optical waveguide or another distal portion of the laser pulse delivery tool as the laser pulse delivery tool is translated relative to the incision during formation of the incision. Such contact and associated friction will lead to shear forces being applied to the incision sidewall tissue. Shear forces that result in deformation of the incision sidewall tissue that exceeds the elastic deformation limit can cause tissue necrosis and associated scar tissue formation.

In the case of an incision formed by a laser pulse delivery tool or mechanism configured to delivery laser pulses to the tissue in a non-contact manner involving free-space laser pulse delivery, an incision that is too narrow may result in clipping of the laser beam by the skin surface and/or incision sidewalls. Such beam clipping can lead to problems such as, but not limited to, incision widening instead of incision deepening, and the accumulation of heat accumulation due to the local fluence being insufficient to reach a threshold for stress-confined impulse tissue ablation.

Accordingly, in some example implementations, an image may be processed to infer a width of the incision (for example, using image processing methods described above for incision localization). The inferred width, along with a known depth of the incision (which may be inferred, for example, based on signals from a depth sensor, or based on a known depth status of a depth control mechanism, as described herein), may be employed, according to pre-determined criteria, to determine whether or not the incision is sufficiently wide. In one example implementation, the criteria may prescribe a minimum width, as a function of incision depth, that is needed to avoid the application of shear forces (between the laser pulse delivery tool and the incision sidewalls) that would result in inelastic tissue deformation. In another example implementation, the criteria may prescribe a minimum width, as a function of incision depth, that is needed to avoid beam clipping of a laser beam by a free-space laser pulse delivery mechanism.

If the incision width is deemed to be insufficient, feedback can be generated to facilitate widening of the incision. Non-limiting examples of different forms of feedback generated in response to a detection of insufficient widening include audible warnings, warnings displayed on a display device, and haptic feedback provided via a haptic actuator (e.g. a piezoelectric device). In other example implementations, the feedback can additionally or alternatively be implemented as control signals that are sent to the tensioning mechanism to increase the applied tension when a state of insufficient incision width is detected.

In some example implementations, images acquired by the imaging camera may be employed to determine whether or not a line of sight exists from the viewpoint of the imaging camera to the bottom of the incision. For example, the presence of a line of sight may be deemed, in some cases, to be sufficient to avoid or reduce the aforementioned problems associated with shear forces applied to the side walls of the incision or beam clipping. For example, a determination of sufficiently of line of sight may be determined by based on a pre-established relationship establishing a minimum width, for a given incision depth, to facilitate sufficient line-of-sight access. In some example implementations, image processing techniques may be employed to classify the images as having a presence or absence of line-of-sight to the incision bottom. A non-limiting example of an image processing method includes a convolutional neural network, PCA image reconstruction or other deep learning algorithm trained on images of incisions that are labeled as having or being absent of line-of-sight to the incision bottom. Alternatively, an optical scanner, such as, for example, an infrared scanner or UV scanner, can be incorporated to acquire images of incision edges that exhibit contrast at the incision edges. In some example implementations, the incision edges can be dyed in order to produce increased contrast in images of the incision edges. In this application, OCT can be used to image the specific depth, strain development to keep within linear limits, and the specific details of the edges at the bottom of the incision. Normally, OCT depth resolution is limited by scatter to a few hundred microns. In this application, with applied tension to open the incision to allow further progression, a clear image to the bottom of the incision is possible to arbitrary depths upon removal of tissue and the scattering source limiting depth resolution.

Although FIGS. 9A-9F show examples of tension control structures that are indirectly supported on the skin surface via the support substrate 300, it will be understood that the tension control structure may alternatively be configured to be directly secured to the skin of the patient, for example, via an adhesive provided on a bottom surface of the tension control structure, or, for example, via one or more ports configured to be interfaced with a suction-generating device (e.g. a pump or syringe).

In some example embodiments, the aforementioned methods involving (i) active tension monitoring and tension control to avoid the application of strain within the incision that would exceed the elastic deformation limit of tissue and (ii) active incision monitoring and tension control to maintain sufficient opening of the incision may be combined and performed in parallel. For example, the tensioning mechanism can be operated in a closed-loop manner based on the images acquired from in imaging camera to apply tension that will not result in inelastic tissue deformation with the incision yet will provide a sufficient depth-dependent incision width to permit line-of-sight visibility of the incision bottom.

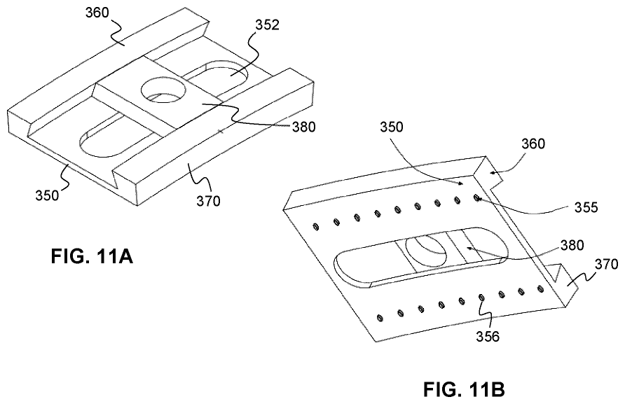

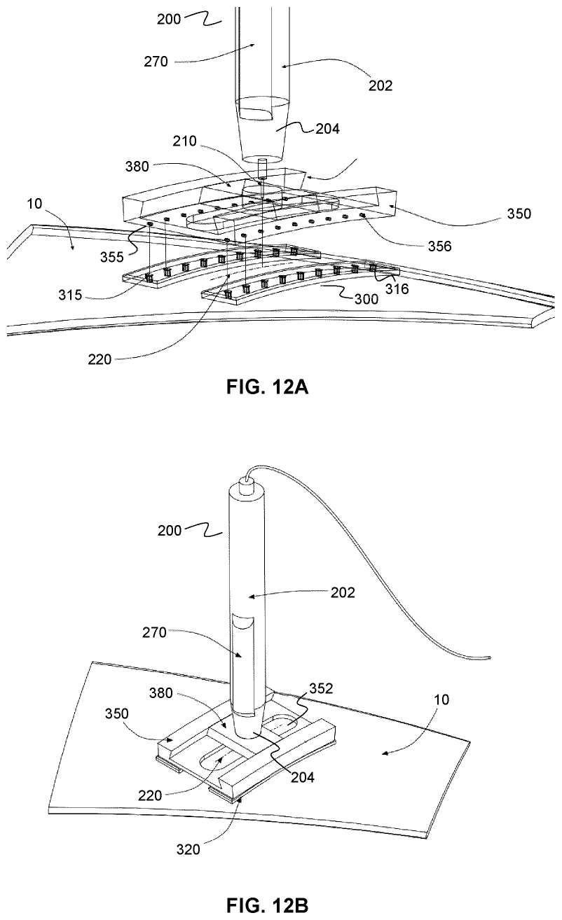

As shown in FIGS. 11A, 11B, 12A and 12B, the support substrate 300 may be employed to provide a support (base) structure for a guide structure 350. The example guide structure 350 shown in the figures is configured to be removably attachable to the support substrate 300, and the guide structure is further configured to receive and guide translation of the laser pulse delivery tool 200 along a prescribed incision path that resides between the first elongate substrate portion 310 and the second elongate substrate portion 320 for forming the incision. The guide structure may include first and second elongate guide portions 360, 370 and a central aperture 352 that provides access to the tissue surface, as shown in FIG. 11A.

In some example implementations, the guide structure 350 may be fabricated to conform to the specific curvature of patient skin surface proximal to the planned incision location. For example, rapid fabrication methods may be used, such as, but not limited to, 3D printing, quick casting, vacuum foaming, sintering. In such a case, the surface profile of the skin surface may be pre-operatively determined using surface scanning methods such as, but limited to, laser radar, structured light, and stereographic imaging.

The laser pulse delivery tool 200 may include one or more first features that engage with one or more corresponding second features of the rigid guide structure 350 to facilitate engagement of the laser pulse delivery tool 200 with the rigid guide structure 350 to permit guided translation of the laser pulse delivery tool 200 relative to the rigid guide structure 350 for forming the incision.

In some example implementations, a tool carriage 380 is provided and configured to support the laser pulse delivery tool 200 relative to the rigid guide structure 350 and to facilitate bi-directional translation of the laser pulse delivery tool 200 relative to the rigid guide structure 350. When the laser pulse delivery tool 200 is received within the tool carriage 380, translation of the tool carriage 380 relative to the rigid guide structure guides the translation of the distal tip of the optical waveguide while maintaining the elongate axis of the optical waveguide substantially perpendicular to the surface of skin in order to facilitate even ablation of the skin layers. Alternatively, a free-space deliver laser pulse delivery tool 200 (having one or more distal optical components suitable for focusing the laser pulses remote from a distal end of the laser pulse delivery tool) may be received within the tool carriage 380, and translation of the tool carriage 380 relative to the rigid guide structure guides the laser pulse delivery tool while maintaining the a distal beam axis associated with the one or more distal free-space optical components) substantially perpendicular to the surface of skin in order to facilitate even ablation of the skin layers.

For example, as shown in FIGS. 11A, 11B, 12A and 12B, the laser pulse delivery tool 200 may be received and supported by the tool carriage 380 that is slidably movable relative to the guide structure 350, such that the incision is formed by sliding the tool carriage 380 relative to the guide structure 350.

The tool carriage 380 may be guided with a tolerance that is less than the width of the laser beam at the distal tip of the optical waveguide 210 (or at the focus of a free-space laser pulse delivery tool).

The tool carriage 380 may include one or more first features that engage with one or more corresponding second features of the rigid guide structure 350 to permit and guide translation of the tool carriage 380 relative to the rigid guide structure 350. FIGS. 12A and 12B illustrate an example case in which a dovetail structure is configured to facilitate slidable translation of the tool carriage 380 relative to the rigid guide structure 350.

As shown in FIGS. 8A-8C and 12A, the support substrate 300 and the rigid guide structure 350 may include respective engagement features such that an intraoperative separation between the first elongate substrate portion 310 and the second elongate substrate portion 320 is enforced by the rigid guide structure 350. The engagement features may include, for example, a female feature and male fixation pin, vacuum cups, and/or other means that can effectively fix the rigid guide structure 350 onto the support substrate 300. For example, as can be seen in FIG. 12A, the rigid guide structure 350 may include a plurality of pins 355, 356 that are received within corresponding holes 315, 316 provided within the first and second elongate substrate portions 310, 320. While the present example embodiments are illustrated with multiple engagement features for each of engagement features 355, 356, 315 and 316, it will be understood that each of engagement features 355, 356, 315 and 316 may be provided as a single respective engagement feature. For example, engagement features 315 and 316 may be linear slots and engagement features 355 and 356 may be rails that are received within slots 315 and 316 to facilitate engagement.

In some example embodiments, the separation between the first elongate substrate portion 310 and the second elongate substrate portion 320 may be initially defined by an alignment member 330. The initial separation between the first elongate substrate portion 310 and the second elongate substrate portion 320 may be different from an intraoperative separation between the first elongate substrate portion 310 and the second elongate substrate portion 320 that is defined by the engagement features 315, 316, 355 and 356 that facilitate engagement of the guide structure 350 with the support substrate 300.

This change in separation between the initial separation and the intraoperative separation may be employed to apply tension across the skin region after attachment of the rigid guide structure 350 to the support substrate 300 and during formation of the incision. For example, if the separation between the engagement features 315 and 316 of the support substrate 300 is less than the separation of the engagement features 355, 356 of the guide structure 350, then the attachment of the guide structure 350 to the support substrate 300 will result in an increased amount of tension applied across the skin surface residing between the first elongate substrate portion 310 and the second elongate substrate portion 320.

One potential benefit of such a configuration is that the application of the tension to the skin tissue is mediated by the guide structure 350 without applying any corresponding tensile or compressive forces to the tool carriage 380. Accordingly, when tension is applied across the central tissue region as a result of the engagement between the guide structure 350 and the support substrate 300, the translation of the tool carriage 380 is unaffected by the applied tension. This in turn avoids the application of excess shear forces to the underlying skin tissue during translation of the tool carriage 380 and the laser pulse delivery tool 200.

In some example implementations, the alignment member 300, and the rigid guide structure 350, and the engagement features may be provided such that a difference between the initial separation and the intraoperative separation results in an applied tension of less than 15-20 MPa. In some example implementations, the alignment member 300, and the rigid guide structure 350, and the engagement features may be provided such that a difference between the initial separation and the intraoperative separation results in an applied tension, within the incision, during formation of the incision, that remains within an elastic deformation limit skin tissue.

In some example implementations, the support substrate 300 and the alignment member 330 may be provided as a set, and multiple sets may be provided, each set having differently-spaced engagement features 315, 316 configured to engage with respective engagement features of a guide structure 350, such that the engagement of the guide structure 350 with one set results in a different amount of applied tension to the skin surface than the engagement of the guide structure 350 with another set.

Although FIGS. 11A, 11B, 12A and 12B show a guide structure that is indirectly supported on the skin surface via the support substrate 300, it will be understood that the guide structure may alternatively be configured to be directly secured to the skin of the patient, for example, via an adhesive provided on a bottom surface of the guide structure, or, for example, via one or more ports configured to be interfaced with a suction-generating device (e.g. a pump or syringe).

In some example embodiments, as illustrated in FIGS. 12A and 12B, the laser pulse delivery tool 200 may include a depth control mechanism 270 for controlling a depth of the distal tip of the optical waveguide 210 (or of a focus of a free-space delivery laser pulse delivery tool) relative to the skin surface 10 or relative to the guide structure 350, tool carriage 380 or support substrate 300. For example, the optical waveguide (or one or more distal free-space optical focusing elements) may be supported by a distal body portion 204 of the laser pulse delivery tool 200, where the distal body 204 potion is extendable, relative to a proximal body portion 202, in a direction parallel to the axis of the optical waveguide 210, via actuation of the depth control mechanism 270. Non-limiting examples of suitable depth control mechanisms include motor assembly to enable extrusion and retraction of the distal body 204. Upon operating depth control mechanism 270, the position of the fiber tip can be controlled. In another example, detectable markings such as conductive coating may be incorporated on the fiber sleeves to give feedback mechanism of the position of fiber tip. These motion controls can include a stepper motor with micron to submicron step sizes to keep translation and contact within the linear elastic limit. Other translation devices include DC motor actuators with optical encoders to accurately advance the screw. In another example, lead screw with accurate incrementation steps may be incorporated into the laser pulse delivery tool to realize axial motion of fiber tip. The depth control mechanism 270 may be manually actuated. Alternatively, the depth control mechanism 270 may be an automated mechanism that is operably coupled to, and receives control signals from, the control and processing circuitry.

In some example embodiments, the depth control mechanism 270 may be controlled according to feedback generated based on signals received from a depth sensor residing on the laser pulse delivery tool, such as sensor 260 shown in FIG. 1B. For example, the depth control mechanism 270 may be actuated in a closed-loop manner according to feedback from the depth sensor such that the depth of the distal tip of the optical waveguide 210 is advanced to maintain or re-establish contact with the tissue within the trough of the incision. The depth sensor may be configured to detect signals dependent on a spatial offset, in a depth direction, between the distal tip of the optical waveguide and a bottom of the incision, where the depth control mechanism is controlled in a closed-loop configuration according to control criteria that employs the signals from the depth sensor.

In some example implementations, the depth control mechanism is controlled according to control criteria dependent on a number of passes of the laser pulse delivery tool along the prescribed incision path. For example, one of both of the laser pulse delivery tool and the guide structure 350 may include a sensor for detecting a number of passes of the laser pulse delivery tool during formation of the incision, wherein said control and processing circuitry is operably coupled to said sensor.

The guide structure 350 may form a reference for determining changes in the depth of the incision as determined based on signals from a depth sensor.

The guide structure 350 may include a feature that mechanically actuates the depth advancement mechanism. For example, the guiding structure may include a protrusion that engages with an actuator tab on the laser pulse delivery tool to actuate the depth advancement mechanism when the laser pulse delivery tool passes by or near the protrusion.

As noted above, in some example implementations, a force sensor may be employed sensing signals dependent on a force applied to said distal tip by the skin tissue during translation of said laser pulse delivery tool, as described above, and the depth control mechanism may be controlled based on the signals detected by the force signal in order to maintain contact between the distal tip and the skin tissue.

After use, the guide structure 350 may be removed from engagement with the underlying support substrate 300, for example, by loosening the engagement connections. The support substrate may be removed from the skin by dissolving adhesive employed to apply the support substrate to the skin surface, or by removing any non-adhesive form of attachment, such as vacuum suction. The removal of the device provides the operator with a clean skin surface and clear incision with precise and un-distorted tissue edges.

In some example implementations, the rigid guide structure 350 may be provided such that the intraoperative separation between the first elongate substrate portion 310 and the second elongate substrate portion 320 is controllable after attachment of the rigid guide structure 350 to the support substrate 300, thereby permitting intraoperative tuning of an amount of tension applied to the skin region. For example, the rigid guide structure 350 may be formed from two components that are rigidly held together by one or more attachment members while permitting a variable separation therebetween.

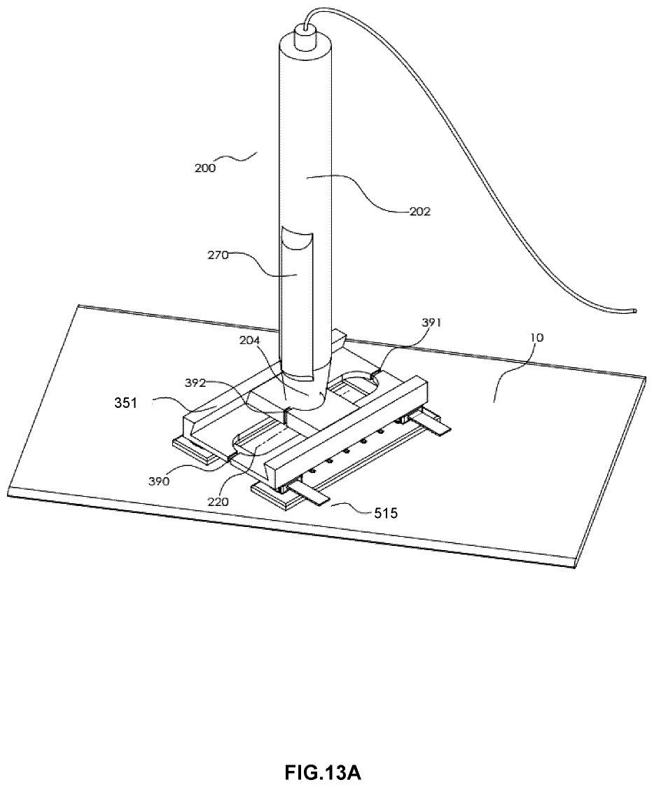

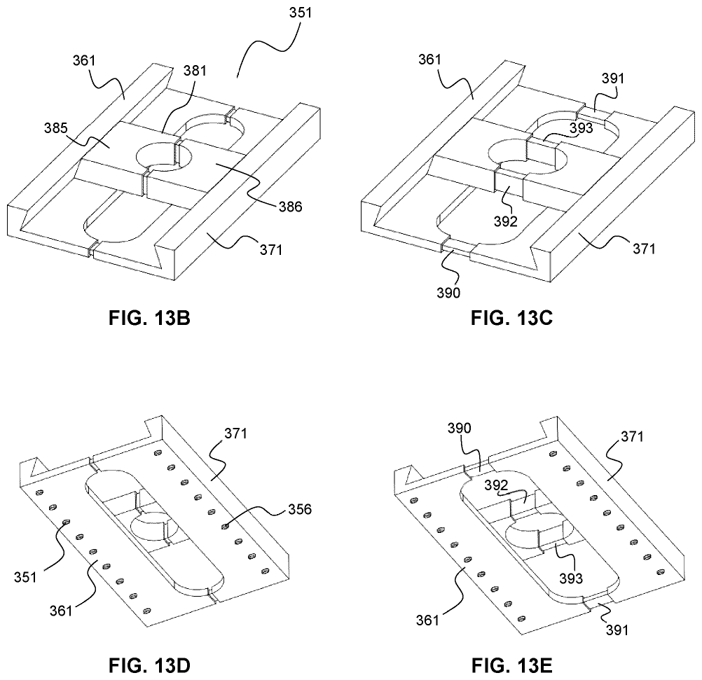

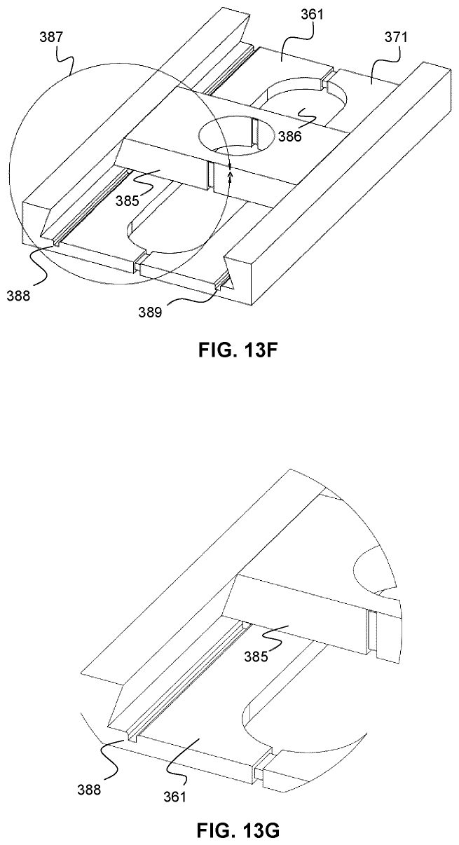

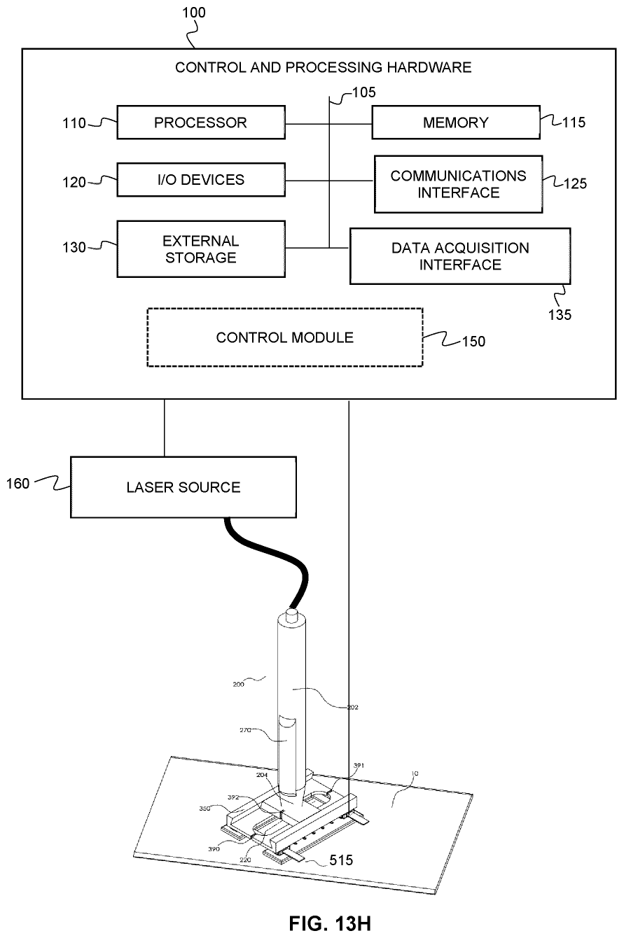

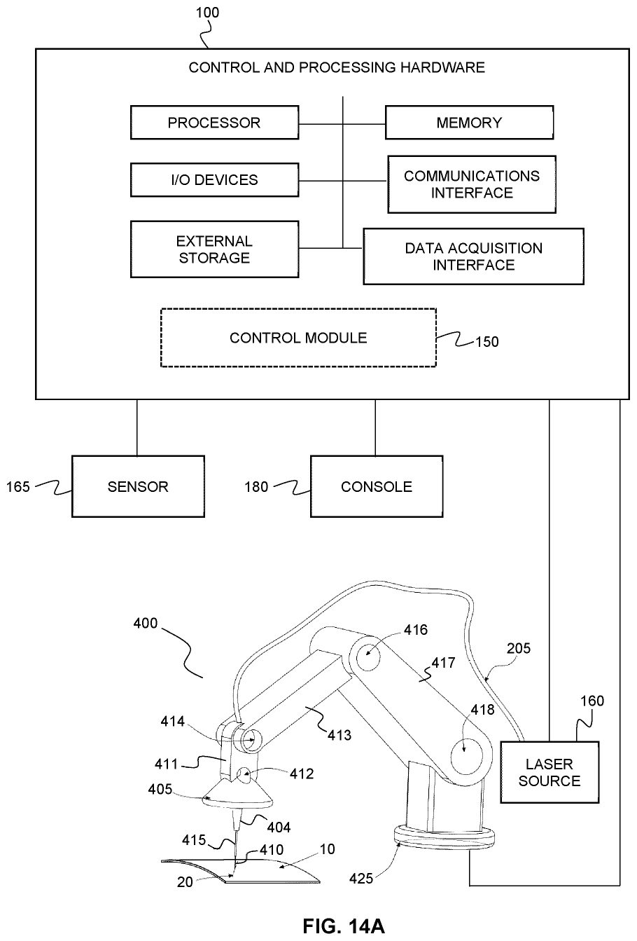

FIGS. 13A-13E illustrate an example embodiment of a guide structure 351 that is secured on the tensioning control structure (having an integrated tensioning mechanism 515) previously described with reference to FIG. 9D-9F. As shown in the figure, an expandable guide structure 351 is provided that includes fastening features configured to fasten the guide structure 351 to the underlying tensioning mechanism such that the pins 355 and 356 (shown in FIG. 13D) can mechanically mate with corresponding features in the underlying tension control structure, such as liner slots 561-564 shown in FIG. 9D.

As shown in FIGS. 13B-13D, the expendable guide structure 351 includes first and second portions 361 and 371 where at least one of the two portions 361 and 371 is slidably extendable relative to expansion members 390,391. For example, at least one of the two portions 361 and 371 may slidably receive, within an internal recess, the expansion members 390, 391, thereby facilitating expansion of the guide structure 351 when tensioning mechanism 515 is actuated to apply tension to the underlying skin. Likewise, the tool carriage 381 includes first and second portions 385 and 386 where at least one of the two portions 385 and 386 is slidably extendable relative to expansion members 392,393. For example, at least one of the two portions 385 and 386 may slidably receive, within an internal recess, the expansion members 392, 393, thereby facilitating expansion of the tool carriage 381 when tensioning mechanism 515 is actuated to apply tension to the underlying skin. The two portions 385 and 386 may be secured in an extended position, for example, via an internal biasing mechanism or via a fixation mechanism (such as a set screw).

In one example implementation, the tool carriage portions 385 and 386 may be configured to be expanded along with the expansion of the first and second portions 361 and 371. For example, as shown in FIGS. 13F and 13G (where FIG. 13G shows a detailed view of region 387 from FIG. 13F), the tool carriage portions 385 and 386 may be fixed longitudinally by means such as, but not limited to, spatial features such as longitudinal grooves 388 and 389 defined within the surface of the first and second portions 361 and 371. In another example implementation, elongate rods, secured to the first and second portions 361 and 371, may respectively extend longitudinally through the tool carriage portions 385 and 386 such that the tool carriage portions 385 and 386 can slide longitudinally relative to the elongate rods as the separation between the first and second portions 361 and 371 is varied. In another example implementation, the tool carriage may include an internal biasing mechanism or fixation mechanism to facilitate expansion.

The tension control structure may include a sensor that is capable of generating a signal dependent on the applied tension. The signal may be processed by the control and processing hardware 100 to determine whether or not the applied tension is likely or expected to result in the generation of forces within the incision that are below the elastic deformation limit of tissue within the incision. FIG. 13G illustrates an example implementation in which a guide structure resides on a tensioning control structure and that includes a tension sensor (not shown in figure) that are connected (or are connectable) to the control and processing hardware 100.

In alternative example embodiments, the guide structure itself may include an integrated tensioning mechanism. For example, the tensioning belt 552 of the integrated tensioning mechanism 515 shown in FIG. 9E may connect the portions 361 and 371 of the guide structure 351 shown in FIGS. 13B-13D, with the tensioning part 554 being integrated into one of the two portions 361 and 371 of the guide structure 351.

It will be understood that any or all of the preceding example embodiments pertaining to the use of a tensioning mechanism, tension sensor, and indirect optical imaging camera for the control of applied tension (for example, to avoid inelastic deformation of tissue within the incision, and/or, for example to a maintain sufficiently wide incision during multi-pass formation of the incision) may be employed in conjunction with, or adapted to, the present example embodiments involving a guide structure having an integrated tensioning mechanism. While many of the preceding example embodiments involve the manual control of a laser pulse deliver tool to form an incision by manual translation of the laser pulse delivery tool following an incision guide (according to feedback suitable for maintaining alignment with the incision guide) or by manually translating the laser pulse delivery tool relative to a guide structure that is secured to the skin surface of the patient, it will be understood that such embodiments are merely provided as examples and are not intended to limit the scope of the present disclosure to embodiments involving the manual translation of a laser pulse delivery tool for the formation of an incision. Indeed, in other example embodiments, the laser pulse delivery tool may be translated, relative to the skin surface, by a robotic assembly, in order to deliver PIRL laser pulses suitable for forming an incision in skin, with or without use of a guide structure secured to the skin of the subject.

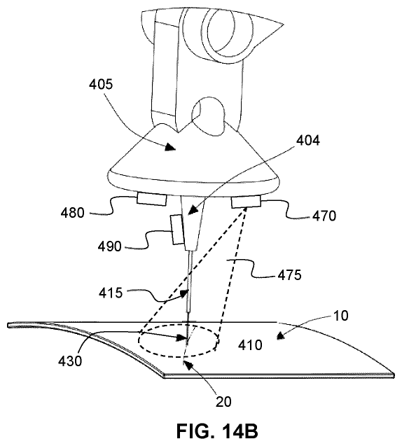

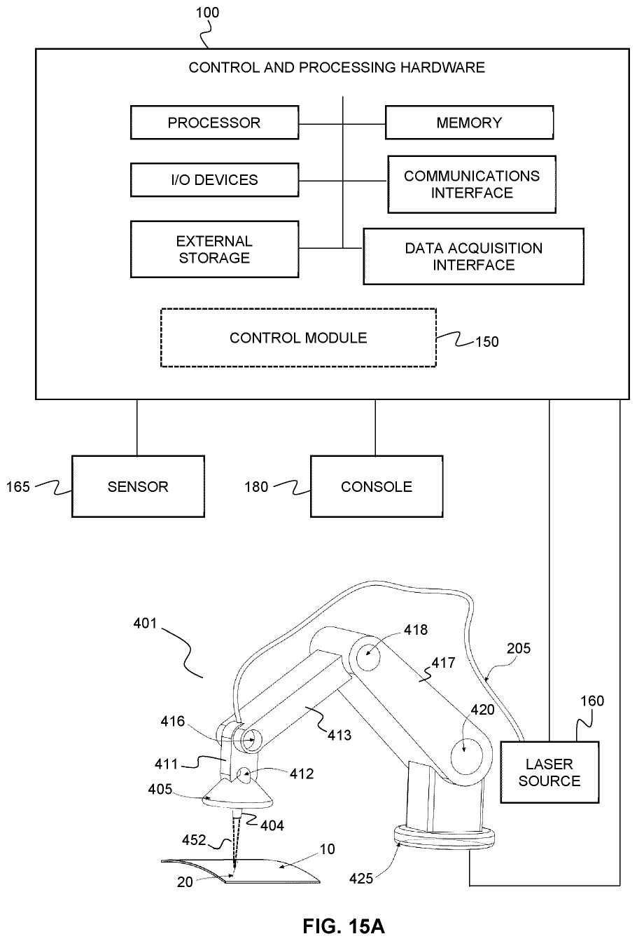

An example of such a system is illustrated in FIG. 14A in which a robotic assembly 400 is employed to deliver the PIRL laser pulses to the tissue surface 10. A detailed view of the robotic assembly 400 is illustrated in FIG. 14B. The robotic assembly 400 is interfaced to the control and processing circuitry 100. As shown in the figure, the robotic assembly 400 may be an articulating assembly including a plurality of joints, such as joints 412, 414, 416 and 418, and links, such as links 411, 413 and 417, and may be supported by a base 425. One or more joints may be actuated by a respective motor (not shown in the figure) that is operatively connected to, and controllable by, the control and processing system 100.

In the example embodiment shown in FIGS. 14A and 14B, an optical fiber 205 is employed to deliver PIRL laser pulses from the laser source 160 to the distal head 405 of the robotic assembly 400. An optical waveguide 410 is supported by the distal head 405 forms a distal functional portion of the robotic assembly 400. As shown in the figure, a portion of the optical waveguide 410 may be cladded with a protective cladding or sheath 415. The optical waveguide 410 is employed to deliver the laser pulses to the tissue surface 10 to form the incision. The optical waveguide 410 may be a distal portion of the optical fiber 205. Alternatively, the optical waveguide 410 may be a separate structure to which the optical fiber 205 is optically coupled (e.g. within the distal head 405 of the robotic assembly 400) for delivery of the PIRL pulses thereto. Various example structural forms of the optical waveguide 410 are described in detail above.

In example implementations in which the optical waveguide 410 lacks rotational symmetry about its distal longitudinal axis, such as, but not limited to, multi-fiber arrays, such as those shown in FIGS. 4A, 4E and 4F, the robotic assembly may include a distal rotational joint suitable for maintaining alignment of the optical waveguide with the incision (e.g. or with an incision guideline).

Although the example embodiment shown in FIGS. 14A and 14B illustrates an integrated implementation in which the optical fiber and waveguide extend from the distal head 405 of the robotic assembly 400, in other example embodiments, the robotic assembly may be configured be attachable to a laser pulse deliver tool and employed to move the laser pulse deliver tool relative to the incision. For example, a distal region of the robotic assembly may include a connection mechanism (e.g. gripping mechanism) that is capable of securing the laser pulse delivery tool.

The control and processing system 100 may include or be connectable to a console 180 that provides an interface for facilitating an operator to control the robotic assembly 400 and/or the laser source 160. The console may include, for example, one or more input devices, such, but not limited to, a keypad, mouse, joystick, touchscreen, and may optionally include a display device.

The robotic assembly 400 may include one or more sensors, such as an imaging sensor 470 and/or a non-imaging sensors 480 and/or 490. In some example embodiments, the robotic system may include an imaging sensor (camera) 470 having a field of view suitable for acquiring video images of the incision during formation of the incision. For example, as shown in FIG. 14B, the distal head 405 of the robotic assembly 400 may include a camera 470 having a field of view 475 that includes the distal tip 430 of the optical waveguide 410. Images acquired by the camera 470 may be employed for a variety of purposes.

For example, images acquired by the camera 470 may be displayed on the console 180 or another display interfaced with the control and processing hardware 100.

In some example implementations, a plurality of imaging cameras may be employed and the image data from the multiple imaging cameras may be employed, for example, to generate three-dimensional images and/or surface data characterizing the skin surface and/or features of the incision. Images from the imaging sensor(s) may be processed using, for example, the example methods previously described herein.

The robotic assembly may include other types of sensors, such as, but not limited to, distance sensors, proximity sensors, force sensors, surface detection/profile scanners, temperature sensors, and collision avoidance sensors. While sensors 470, 480 and 490 are shown supported by the distal head of the robotic assembly 400, in other example implementations, one or more sensors may additionally or alternatively be located on (e.g. supported by) another portion or region of the robotic assembly 400. In addition, one or more sensors may be remote from the robotic assembly 400.

In some example implementations, the robotic assembly may be further configured to deliver an optical coherence tomography sample beam onto to skin tissue within the incision and to collect light scattered from the tissue. This collected light may be combined with a reference beam to generate one or more optical coherence tomography scans or images characterizing the tissue within the incision.

In some example embodiments, signals obtained one or more sensors supported by the distal head 405 of the robotic assembly 400 may be processed to generate one or more feedback measures for controlling one or both of positioning of the robotic assembly 400 and delivery of laser pulses by the laser system (e.g. control of the pulse repetition rate) during the formation of the incision. For example, images acquired by the camera 470 may be processed to infer a state of misalignment with an incision guideline, such as an artificial incision guideline or an incision guideline associated with edges of a partially-formed incision), and control signals may be generated and sent to the robotic assembly 400 for correcting a state of misalignment.

In other example implementations, one or more portions of the robotic assembly 400 may include fiducial markers that are trackable with a tracking system. For example, the distal head 405 may include at least three trackable passive or active fiducial markers that are detectable with an optical tracking system having a pair of tracking cameras, where images obtained from the tracking cameras can be processed to determine a real-time position and orientation of the distal head 405. In such a case, the images from the tracking camera may also be processed to determine the real-time location of the incision. Positional (and optionally orientation) feedback for maintaining the position (and optionally orientation) of the distal head 405 relative to the incision may be generated based on the real-time knowledge of the position and orientation of the distal head 405 and a tracked incision guide (e.g. an artificial guide or an incision guide determined based on tracking of edges of the incision formed from one or more previous passes of the laser beam, as described previously).

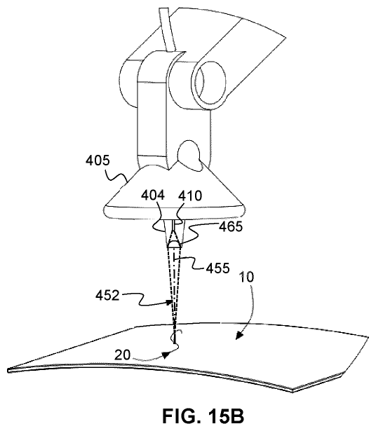

FIGS. 15A and 15B illustrate, respectively, an example system and example robotic assembly in which the laser pulses are delivered via free space optics. As shown in the figures, the distal head 405 includes one or more optical components (e.g. lens 465 in FIG. 15B) that are employed to focus laser pulses emitted by the optical fiber 205 (i.e. emitted at distal tip 410) onto the tissue surface 10. The focused laser beam is shown at 452 and the beam axis 455 is shown in FIG. 15B. While FIGS. 15A and 15B illustrate an example implementation in which the laser beam is scanned laterally relative to the tissue surface via translation and/or rotation of the robotic assembly 401, the robotic assembly may alternatively include a scanning mechanism (e.g. a galvanometer scanner including scanning mirrors and motors) configured to scan the beam of laser pulses relative to the incision in the absence of translation/rotation of the robotic assembly. In such a case, the robotic assembly may be positioned to a suitable location via actuation of the joints and the scanning mechanism may be employed to scan the beam of laser pulses, relative to the robotic assembly, on the skin surface for formation of the incision.

Although the example robotic assemblies shown in FIGS. 14A, 14B, 15A and 15B employ rotational joints to facilitate control of the three-dimensional positioning of the beam of laser pulses relative to the skin surface, in some example implementations, the robotic assembly may include at least one linear translation stage. The linear translation mechanism may be the distalmost actuation mechanism of the robotic assembly and actuated to translate the beam of laser pulses during formation of the incision.

In some example implementations, a guide structure, such as the guide structure illustrated in FIGS. 11A, 11B, 12A and 12B, may be employed to guide translation of a distal portion the robotic assembly, or to guide a laser pulse delivery tool held by the robotic assembly (for contact-based or free-space-based delivery of laser pulses). In such implementations, one or more surfaces of the guide structure may be employed as reference surface for determining, via a depth sensor, a depth measure associated with the depth of a functional distal end of the robotic assembly relative to the incision. In some example embodiments, a tensioning mechanism may be employed to apply a controlled amount of tension during delivery of the laser pulses by the robotic assembly. For example, a tension control structure may be employed to apply tension to the skin surface during robotic delivery of the laser pulses, such as, but not limited to, the example tension control structure shown in FIGS. 9B-9E, or, for example, a guide structure having an integrated tensioning mechanism or configured to engage with a tensioning mechanism, such as, but not limited to, the example guide structure shown in FIG. 13A.

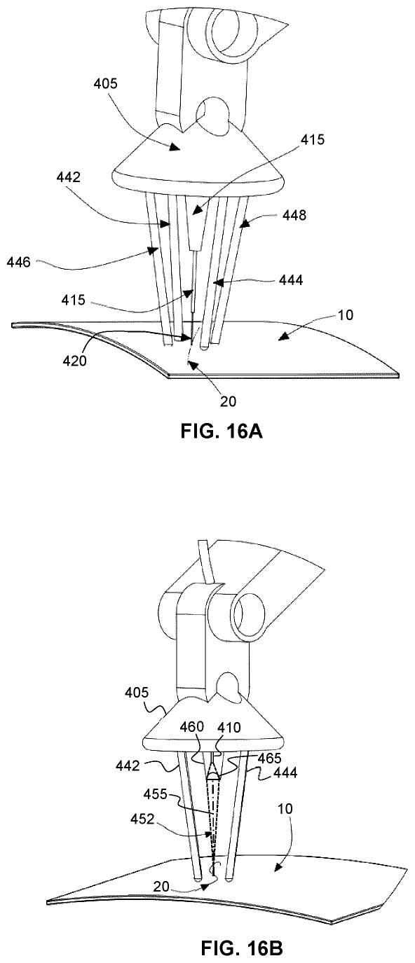

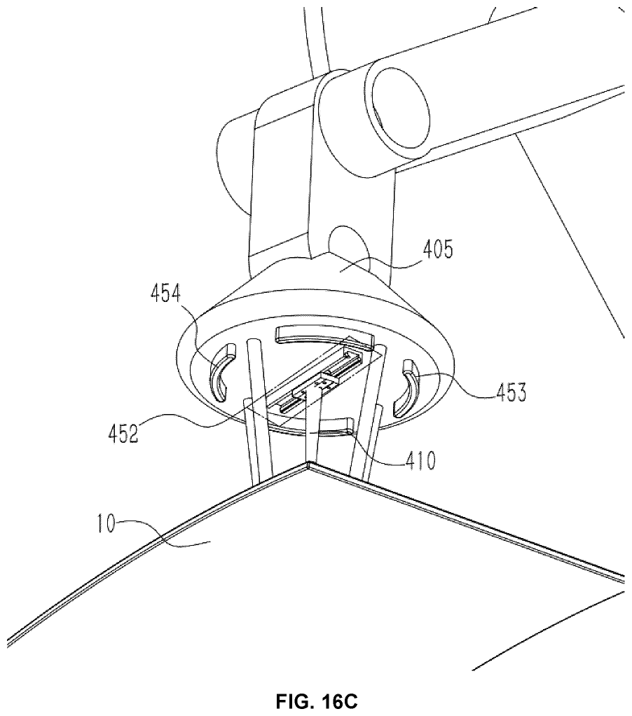

In other example embodiments, the robotic assembly may include a tensioning mechanism that is controllable to apply a controlled amount of tension to the skin surface during formation of the incision. FIGS. 16A and 16B illustrate example implementations of robotic tensioning mechanisms. As shown in FIG. 16A, a plurality of tensioning members 442-448 extend from the distal head 405 of the robotic assembly 400. The tension control members may grip to the skin by gripping mechanisms include, but not limited to, frictional contact, grippers, and vacuum suction.

In some example implementations, the distal head 405 may include a translation mechanism 452 (e.g. a lead/ball screw, rack-and-pinion or other suitable linear actuator and associated motor) for actuating translation of the distal optical waveguide 410 along the incision, relative to the tensioning members 442-448, during formation of the incision. The distal head 405 may also include a second translation mechanism for varying a depth of the distal end of the optical waveguide, relative to the tensioning members 442-448, during formation of the incision.

In other some example implementations, one or more sensors, such as, but not limited, to a hall effect sensor, infrared positioning sensor, or piezo sensor, can be incorporated into the distal head 405 (shown schematically in the figure at 453 and 454). Signals from one or more of such sensors can be employed to provide control and feedback of cutting motion.

Referring again to FIGS. 16A and 16B, the tensioning members 442-448 have a length suitable for contacting the skin surface during the formation of the incision 20 as the depth of distal end of the optical waveguide 410 is varied over the depth of the incision. The distal optical head also includes a tensioning mechanism that is controllable to vary the separation between adjacent tensioning members on opposing sides of the incision 20 in order to apply tension across the incision. For example, the tensioning mechanism may be actuated to vary the separation between tension control members 446 and 444, and between tension control members 442 and 448. Non-limiting examples of tensioning mechanisms include incorporating a gripping mechanism such as suction cup or friction pads to the tip of control members, such that the opening of tension control member in the direction perpendicular to the incision edge will result in pulling the incision edges away from each other. The tensioning members may also be employed to apply a force on the skin to assist with opening the incision edges. Motorized ball/lead joints between distal head 415 and tensioning members can be used to control relative motions. In some example implementations, a machine vision system (e.g. as described above) may be employed to control the amount of tension applied to the incision to achieve the minimal number of shearing forces on the incision bed.

Although FIG. 16A illustrates an example tensioning mechanism implementation involving four finger-like contact actuators, it will be understood that the tensioning mechanism may take on other forms in alternative example implementations. For example, FIG. 16B illustrates an example implementation in which an example tensioning mechanism includes only two contact actuators 442 and 444. This figure also illustrates an alternative beam delivery configuration in which at least one distal free-space optical component (e.g. optical component 465) is employed to focus the beam of laser pulses 452 to a remote location along a beam delivery axis 455. While the tensioning control mechanism is illustrated as employing elongate rods as contact actuators, it will be understood that the contact actuators may take on a wide variety of shapes.

The tensioning mechanism may be employed to deliver a controlled amount of tension during formation of the incision. The robotic assembly may include a sensor that is capable of generating a signal dependent on the applied tension. The signal may be processed by the control and processing hardware 100 to determine whether or not the applied tension is likely or expected to result in the generation of forces within the incision that are below the inelastic deformation limit of tissue within the incision, to ensure linear elastic response of the tissue without damage to cell structure of the tissue.

As described above, although the tension applied to the skin surface by the tensioning mechanism may be different than the tension applied within the center (bottom) of the incision, a relationship may be established between the signal generated by the sensor and the internal strain that is generated within tissue residing at the center of the incision. The relationship may be determined, for example, via simulations and/or experiments. In the latter case, the strain experienced by the tissue residing at the center (bottom) of the incision may be inferred via the processing of overhead images of the incision, as explained in further detail below. The relationship may be expressed in a continuous or discrete form. For example, the relationship may be defined as a signal threshold beyond which the applied strain within the incision (e.g. at or near the center/bottom/vertex of the incision) is expected to exceed the inelastic deformation limit, or is expected to be at risk of exceeding the elastic response limit (e.g. is associated with a margin or guardband).

Any of the example methods for indirectly measuring or inferring strain applied within the incision by a tensioning mechanism, as described above with regard to the tension control structure, may be employed in example embodiments involving a robotic assembly configured for beam delivery and tension application, such as optical image-based methods. The preceding example methods involving the generation of feedback based on a detection of the applied tension exceeding or risking exceeding the elastic limit of the tissue within the incision, and use of such feedback to control the tensioning mechanism to reduce the applied tension, may also be implemented in the present robotic embodiments. Furthermore, the preceding example methods involving the generation of feedback based on a detection of a sufficiency or insufficiency of width of the incision, and/or a sufficiently of line-of-sight visibility of the incision bottom, and use of such feedback to control the tensioning mechanism to vary the incision width, may also be implemented in the present robotic embodiments.

Although the example robotic beam delivery embodiments illustrate the formation of a linear incision, it will be understood that the scope of the present disclosure is not intended to be limited to the generation of linear (e.g. straight line or curved) incisions, and that other incisions with multiple intersecting incision lines may be generated in the alternative. For example, in some example implementations, the robotic assembly may be controlled to form an incision having an “H” pattern to form tissue flaps, which can be beneficial to facilitate the insertion of large surgical tools insertion. This H-pattern can likewise be conserved with the cut edges of the tissue being brought into close registration, with spatial offsets of less than 100 microns, to facilitate healing without visible scar tissue formation.

In some example implementations, the robotic assembly be employed to deliver a laser tissue welding beam onto the incision after its closure for laser tissue welding. Suitable sources for laser welding would have strong absorption in the tissue or bio-solder. Since hemoglobin absorbs strongly in the visible region, Argon lasers, some diode lasers, and frequency doubled Nd:YAG lasers can be used for this purpose. IR lasers would absorb in the water containing tissue. A PIRL laser could be used, but energy/pulse, the scan time or repetition rate would have to be reduced to create and accumulated thermal effect sufficient to melt the bio-solder. In some example implementations, same optical system that is employed to deliver the laser pulses for incision formation may be employed to deliver the laser welding beam. In other example implementations, an additional optical-fiber-based or free-space optical system may be integrated with the robotic assembly for delivery of the laser welding beam along the incision path, welding the skin surface together with minimal tolerance to achieve closure of a incision without inducing substantial scar tissue formation.

Although the preceding example implementations have been described with reference to the use of PIRL laser pulses (satisfying the aforementioned PIRL-based pulse conditions) for the formation of an incision, it will be understood that the systems, devices and methods described herein are not intended to be limited to PIRL-based implementations. For example, other laser systems may be employed in the alternative, such as, but not limited to, <100 ns Tm:Fiber or Ho:YAG lasers with a laser wavelength of approximately 2.0 μm can achieve stress confinement below the ionization threshold, despite their relatively longer absorption depths of 10-100 deeper than PIRL processing in the 3 μm range. The additional energy required for such a laser is proportional to the change in absorption depth. A short-pulsed CO₂ laser around 9.3 μm would similarly achieve stress confinement with a <100 ns pulse duration.

In other example implementations, the aforementioned example workflows and conditions for achieving tension within the elastic limit may be beneficial for laser-based surgeries using femtosecond lasers (pulse durations shorter than a picosecond) in which plasma formation at high peak powers is used to localize the energy to submicron depths. The short pulse duration in the femtosecond laser case, discounting ionizing radiation effects, leads to tissue removal under full stress confinement of the energy such that enabling advancement of the laser cutting tool without incurring inelastic deformation of the tissue, and thus without resulting in cell damage, and without triggering a signaling cascade for the healing response that would otherwise lead to excessive fibroblast formation and scar tissue. However, it is noted that ionizing radiation is known to delay healing and it can be desirable to avoid such potentially mutagenic form of energy absorption in the surrounding tissue.

Moreover, while the preceding example embodiments relate to systems, devices and methods for the formation of an incision, other example implementations one or more devices may be employed during one or more intraoperative and optionally post-operative steps, as further described below. It will be understood that the although the following systems, devices and methods, may be particularly well suited for facilitating the opening and closure of incisions formed by laser pulses, in particular, PIRL-based laser pulses according to the aforementioned PIRL-based pulse conditions, the scope of the following embodiments is not intended to be limited to PIRL-based laser incisions or even incisions formed by lasers in general, and may be employed for the opening and closure of incisions formed by any method.

In some example embodiments of the present disclosure, an incision protection device is provided that facilitates the protection of the edges of an incision while opening and closing an incision. In the absence of such a device, the opening of an incision (after having formed the incision), for example, to facilitate the insertion of a surgical tool (e.g. a laparoscopic or endoscopic tool), or for example, to permit visualization of internal tissue or organs, may result in the deformation of tissue beyond its elastic limit.

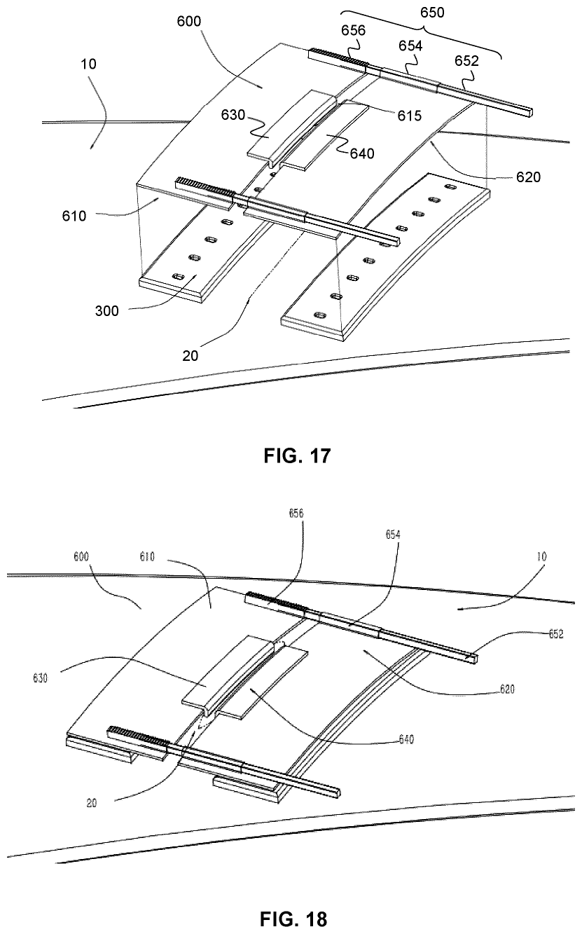

FIGS. 17-21 illustrate an example implementation of an incision retraction structure 600 that can be employed to protect the edges of an incision when opening and closing an incision. The example incision retraction structure 600 includes first and second lateral members 610, 620 that are positioned on opposing sides of the incision 20, such that the incision is accessible between the first and second lateral members 610, 620. The first and second lateral members 610, 620 are removably secured, either directly or indirectly, to the skin surface 10.

As shown in the figures, the incision retraction structure 600 includes a retraction mechanism 650 that can be actuated to vary the separation between the first and second lateral members 610, 620, thereby opening or closing the incision. The retraction mechanism 650 is secured to the first and second lateral members 610, 620. One or more components of the incision retraction structure 600 may be shaped to conform to the curvature of the skin surface and may be flexible (e.g. formed from a flexible material or a flexible mechanical structure).

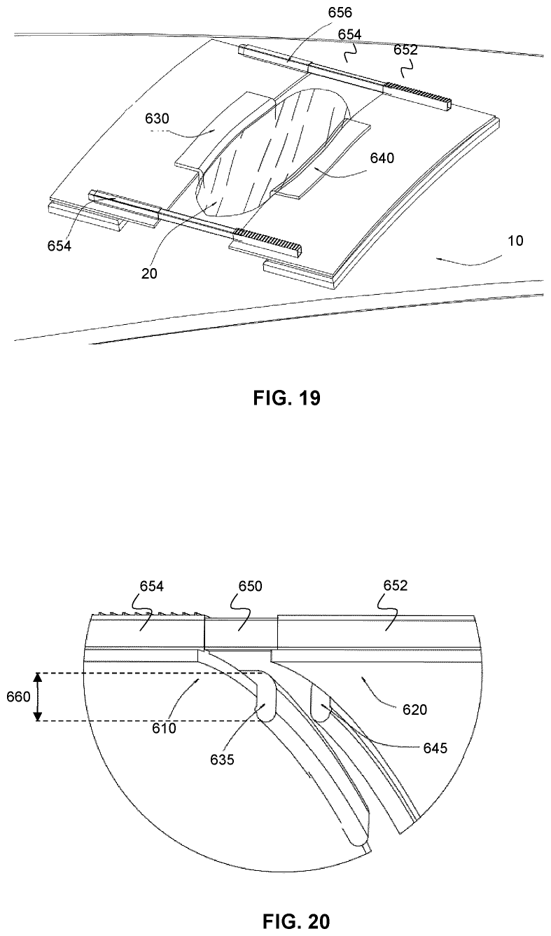

The tissue retraction structure 600 may be initially secured relative to the skin surface in an initial configuration shown in FIGS. 17 and 18. As a consequence of the attachment (either direct or indirect) of the lateral members 610, 620 to the skin surface, actuation of the retraction mechanism 650 to increase the separation between the first and second lateral members 610, 620 results in the opening of the incision, as shown in FIG. 19. The retraction mechanism 650 may also be actuated to close the incision 20, returning the incision to the state shown in FIG. 18.

In some example implementations, the retraction mechanism 650 may be loosened to permit an operator to open the incision using another tool (e.g. a convention surgical tool), and the retraction mechanism 650 may be tightened when the incision is opened. The retraction mechanism 650 may be made of flexible material that allows the mechanism to deflect in small amount to compensate uneven skin or skin curvature.

In the example embodiment presently illustrated, the retraction mechanism 650 includes a tension rod 652 that is rigidly secured to a guide sleeve 654 (such that the tension rod 652 does not slide relative to the guide sleeve 654), and a ratcheting recess 656. The ratcheting recess 656 includes internal corrugations that are configured to mechanically mate with and corresponding external corrugations on a distal portion of the tension rod 652. To release the tension of this mechanism, a release mechanism may be incorporated with the retraction mechanism (e.g. with the ratcheting recess 656) such as, but not limited to, a push-release, pull-release, or press-release mechanism.

Other non-limiting examples of tensioning mechanisms include ziplines, pulling tabs, and threaded mechanisms. While the figures illustrate an example implementation in which two tensioning mechanisms are provided (one on each longitudinal side of the incision), other example implementations may employ a single tensioning mechanism.

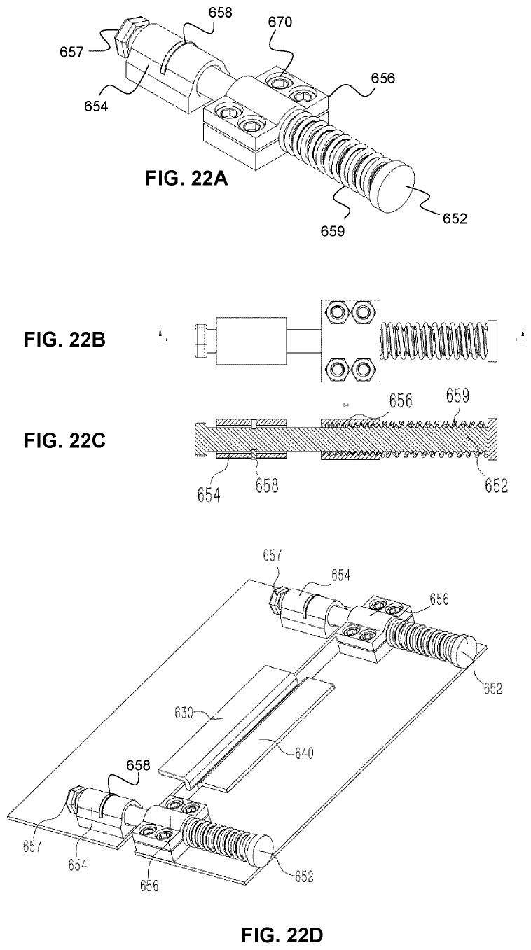

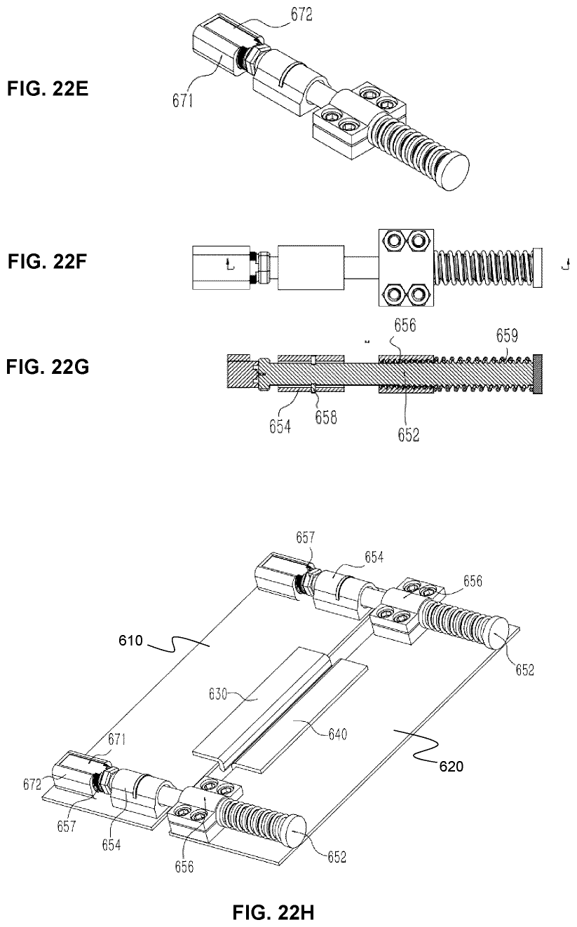

FIGS. 22A-22D and 22B illustrate an example implementation of a retraction mechanism 650 in further detail. In this example implementation, the tensioning rod 652 includes a threaded external corrugation on the distal portion, which mechanically mates with the threaded internal of the ratcheting recess 656. The tension rod 652 is rigidly secured inside the guide sleeve 654 with the retaining ring 658 to restrict axial movement and allowing circumferential rotation with respect to guide sleeve. To open the incision, the knob 657 located on the proximal end of the tensioning rod can be turned. Turning the knob provides precise control of tension. A release mechanism (e.g. a quick release) may be incorporated into the ratcheting recess 656. In this specific example, loosening the screws 670 will open up the ratcheting recess 656 and loosen the internal thread, thereby facilitating motion of the tensioning rod 652 freely inside the ratcheting recess 656, releasing any existing tension. In addition, the release of the retaining ring on the guide sleeve will also release any existing tension.

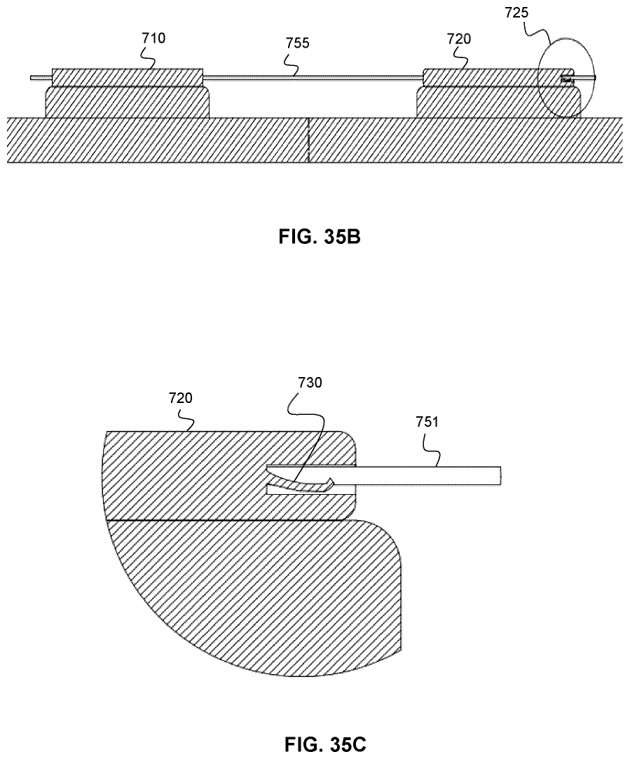

It is noted that turning the tensioning rod counterclockwise will pull edges together even further then its original state, generating negative pressure along the incision edges. By placing a wound protection device prior to the removal of the retracting mechanism 650, this negative pressure can be maintained during scar healing. The wound protection device can be based, for example, on the incision closure structure 700 described in FIG. 35 or variations thereof, or can take on other forms, such as, not limited to, wound closure tapes such as Dermabond® by Ethicon or Steri-Strip™ from 3M.

In other example implementations, and as described further below, the retraction mechanism 650 may include a biasing component, such as a spring or other elastic biasing device. For example, a spring mechanism may be incorporated with the tensioning rod 652, as shown in FIGS. 22A-22H. Upon operating the release mechanism, the incision is closed with the relief of tension resides in the retraction mechanism 650. In other example implementations, the retraction mechanism may include a motor or other drive mechanism that is operatively coupled and controllable by the control and processing circuitry.

FIGS. 22A-22D illustrate an example of the above-described optional biasing components. The distal end of the tensioning rod 652 includes a spring 659. As the tensioning rod 652 rotates inside the ratcheting recess 656, the tensioning rod 652 will be retracted from the ratcheting recess 656, thus compressing the spring 659. With the tensioning rod 652 retracted and the incision opened, a restoring force is stored within the spring 659. During closing of the incision, either by rotation of the tensioning rod 652 or using the release mechanism, the spring 659 will release its restoring force in the process, helping to smooth out the restoration of the initial state. In addition, the spring 659 can be incorporated with a sensor to provide a measure tension measure that is associated with the tension applied to the skin.

In some example implementations, the retraction mechanism is manually actuated to open and/or close the incision. In other example implementations, the retraction mechanism may be actuated in an automated manner, for example, by an actuator supported by a robotic arm. For example, the tension rod 652 of the retraction mechanism 650 can be jointed with a motor assembly, in which the motor assembly be controlled by a user interface. Upon setting up the desired incision length and opening width, the tension rod 652 can move autonomously with the control of motor.

As discussed in the previous paragraph, in one example retraction mechanism, the turning of the tensioning rod can control tensioning. A motorizing component such as servo or different types of motor can be mechanically mate with the proximal end of tensioning rod. Meanwhile, feedback mechanism such as IR/force/distance sensors along with controllers can be incorporated to either or both ends of the tensioning rod to provide information of the state of tension. Simulation results described in a later paragraph can be used to construct the relationship between distance/force/rotations and actual tension experienced by the skin, thus preventing plastic deformation of incision corners.

An example implementation of the above-described motor-coupled retraction mechanism is shown in FIGS. 22E-22H. The motor unit 671 place inside motor and sensor holder 672 can be positioned on the upper surface of the lateral member 610. The motor unit actuator is secured to the proximal end of tensioning rod 652, thereby providing rotation motion and tension control.

As shown in the figure, each lateral member includes a respective incision protection feature 630, 640 that are configured to protect the incision during opening and closing of the incision under actuation of the retraction mechanism, and during an intervening surgical procedure during which the incision is retracted. The incision protection features 630, 640 may be secured to respective lateral members, as shown in the figures, or may be integrally formed with the respective lateral members.

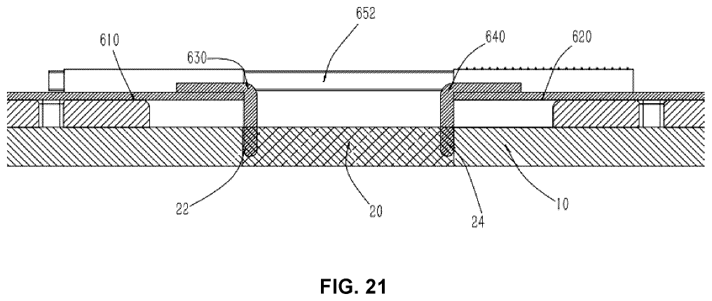

As shown in FIGS. 20 and 21, each incision protection feature 630, 640 includes a respective protective lip 635, 645 that is angled downwards (e.g. perpendicular) relative to the lateral members (i.e. relative to a plane defined by the lateral members, or alternatively relative to a local plane associated with the skin surface). When the incision retraction structure 600 is initially secured relative to the skin surface, as shown in FIG. 18, with the incision in a closed state, the tissue retraction mechanism 650 is configured such that the protective lips 635, 645 are adjacent, either contacting each other or separated by a small gap (as shown in the figure), with a sufficiently narrow separation such that the protective lips reside within the incision without applying a compressive force to the incision edges that would result in inelastic deformation.

Each protective lip 635, 645 has a projection length, along its direction of projection, as shown at 660 in FIG. 21, that is sufficiently long such that when the incision retraction structure 600 is secured in this initial state relative to the skin surface, with the protective lips 635, 645 residing within the incision and the incision being closed, at least a portion of the sidewall of each incision edge is adjacent to and/or contacting a respective protective lip, such that each protective lip extends into the incision toward the incision and covers a respective incision edge.

As the retraction mechanism 650 is actuated to open and close the incision, the protective lips 635, 645 cover and protect the incision edges. The protective lips also protect the incision edges during intervening surgical steps, for example, when inserting and employing surgical tools within the incision.

Moreover, since the incision is open via the application of tension that is mediated through contact (direct or indirect) between the lateral members 610, 620 and the skin surface 10, the incision is opened via the application of a global strain field mediated by the applied tension to the skin surface, thereby reducing the amount of tension or compression that is applied to the incision edge during opening and closing of the incision. Accordingly, the forces applied to the incision edges during opening and closing of the incision can be maintained within the elastic deformation limit. This approach can be contrasted with conventional retraction devices in which the incision is retracted via contact with the incision edges and the application of compressive forces to the incision edges, which can easily exceed the elastic limit, leading to inelastic deformation, and cause scar tissue formation through tissue necrosis.

The figures illustrate an example implementation in which the support substrate 300 is initially secured to the skin surface of the subject and the first and second lateral members 610, 620 are indirectly secured to the skin surface via attachment to the support substrate 300 (e.g. via engagement features 315 on the support substrate 300 and corresponding engagement features (not shown) on the first and second lateral members 610, 620, or via an adhesive). This illustrated embodiment is beneficial in that the same support substrate 300 may be employed both during the formation of the incision (e.g. via support of a guide structure) and subsequently to support the incision retraction structure 600 during subsequent steps of a surgical procedure. Alternatively, the first and second lateral members 610, 620 may be removably adhered directly to the skin surface via a suitable adhesive as described above.

In some example implementations, the retraction mechanism 650 may be configured to mechanically limit the opening of the incision, thus protecting the incision ends from tearing. The limit of expansion may be determined as the maximal expansion of the incision that does not cause tearing as a consequence of forces that are concentrated at the incision ends. For example, mechanical stop can be designed on the distal end of tensing rod 652. In this manner, the opening of the incision can be limited to the point where the distal end of the tensing rod 652 is fully retracted into the sleeve. In another example implementation, a spring/string can be incorporated within the retraction mechanism which could be in loose/coiled status. In such a case, the opening of the incision will pull out the spring/string, and the opening distance would be limited by the maximum distance that the spring/string can extend.

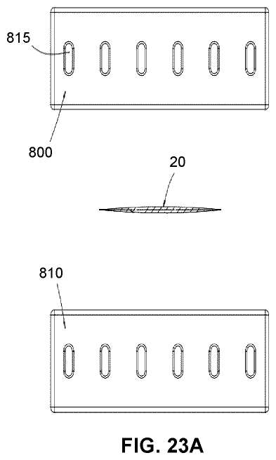

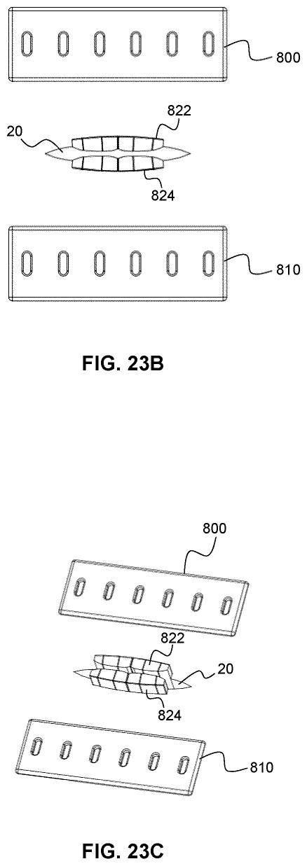

FIGS. 23-27 illustrate another example incision retraction structure suitable for forming a rounded incision opening that facilitates the insertion of, for example, cylindrical tools, trocars, and ports, while applying tension to incision edges that maintains tissue deformation within the elastic region, thus enabling the formation of incisions with the reduction, minimization or prevention of visible scar-tissue formation. Such an example implementation may be beneficial in facilitating an endoscopic incision during minimal invasive surgeries such as abdominal minimal invasive surgeries.

Referring now to FIG. 23A, after having formed an incision, for example, according to any of the preceding example embodiments, lateral support members 800 and 805 are secured on either side of the incision 20. The lateral support members 800 and 810 may be adhered directly to the skin (for example, using an adhesive or suction), or, for example, secured to a support substrate that had been previously adhered to the skin surface (e.g. such as support substrate 300 shown in FIGS. 8A-8C. As shown in the figure, each lateral support member includes at least one fixation feature 815, such as a hole, slot or protrusion.

As shown in FIGS. 23B and 23C, a flexible edge protection structure 822, 824 is positioned inside the incision opening 20. As described below, tension can be applied to the flexible edge protection structure 822, 824 to facilitate the opening of the incision. The flexible edge protection structure 822, 824 may be provided according to a wide range of shapes and configurations. While the flexible edge protection structure may be a single structure, the present figures illustrate an example implementation in which the flexible edge protection structure 822, 824 is provided in a modular structure. In other example implementations, a single (e.g. monolithic) structure can be used for the same purpose. Due to the variety of thickness of skin, shape of opening and incision width, structures of different sizes can be provided with remodeled parameters. Furthermore, rapid additive manufacturing methods can be employed for the fabrication of customized non-modular edge protection structure. Fabrication methods can include but are not limited to: bio-polymer 3D printing, nitinol braced fixture, quick silicon casting, etc.

As shown in FIGS. 23A and 23B, the flexible edge protection members 822, 824 can be inserted into the incision 20 after minimal manual spreading, allowing the distal end of the members to be inserted. The flexible edge protection members 822, 824 may then be pushed to fit inside. Meanwhile, anti-friction surface finishes of the flexible edge protection members 822, 824 and round edges of the flexible edge members will allow a smooth insertion without tearing and damaging the incision edge.

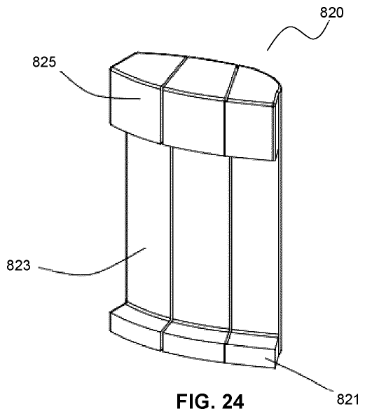

FIG. 24 illustrates shows one example modular sub-structure 820, shown absent of a tensioning belt. The distal end 821 is textured to grip under the skin layers. The intermediate section 823 may be an anti-friction surface that will press on the cross section of the incision as the sub-structure 820 is pulled in the direction perpendicular to the incision. The inner and outer surface of this part may be coated with a low-friction material, such as polytetrafluoroethylene (Teflon®) that reduces tear and wear during insertion, retrieval and operation. The proximal end 825 may be textured to grip on the skin surface.

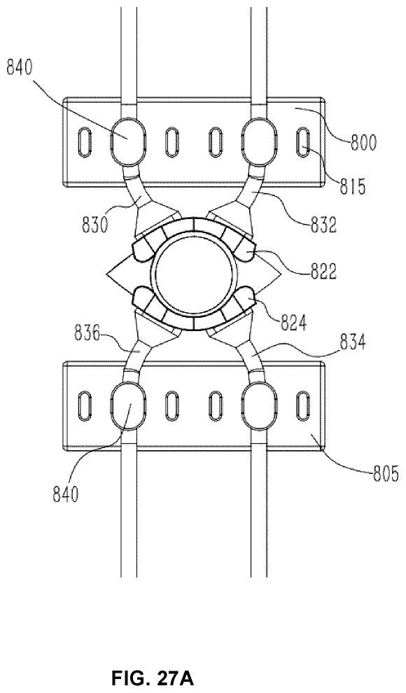

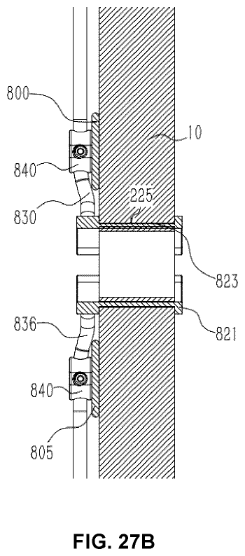

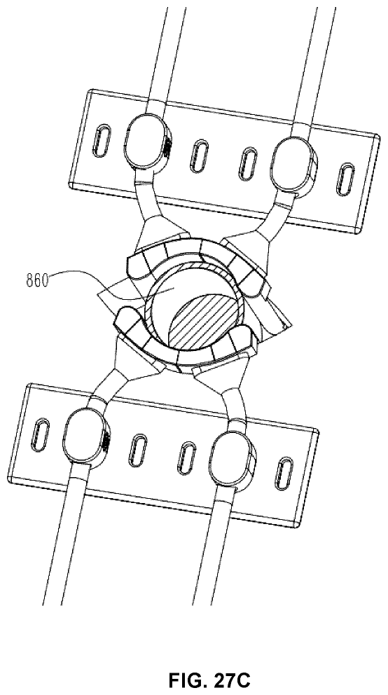

As shown in FIG. 27A, the tensioning belts 840, 842, 844 and 846 are received within respective belt fixation devices 840, 842, 844 and 846. The belt fixation devices 840, 842, 844 and 846 are secured to the lateral support members 800 and 805. For example, the belt fixation devices may be provided in the form of plugs that are fitted into the respective recessed 815 formed within the lateral support members (as shown in FIG. 23A). For example, this can be achieved by a friction fit between a male plug and a female aperture, or, for example, through another temporary attachment mechanism (such as an attachment mechanism using a fastener, such as a set screw, or a latch or clamp). The attachment mechanism is sufficient to ensure the secure attachment of the belt fixation devices relative to the underlying structure during the surgical procedure. to ensure sufficient control of tension for incision edges.

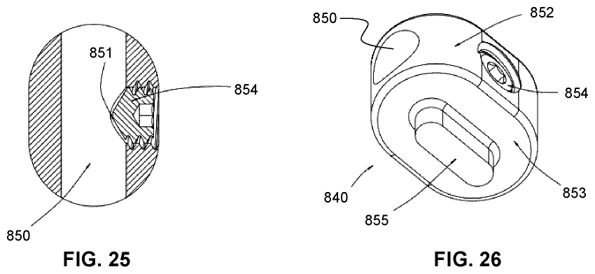

A detailed view of an example belt fixation device 840 is shown in FIGS. 25 and 26. As can be seen in the figures, the belt fixation device 840 includes, in its side 852, a channel 850 configured to slidably receive a respective tensioning belt. The belt fixation device 840 includes a tensioning mechanism suitable for applying tension to the tensioning belt. The figure shows an example tensioning mechanism in the form of set screw 854 that can be adjusted to contact the tensioning belt (at 851) within the channel 850 and thus maintain a level of tension applied to the tensioning belt. In alternative example implementations, the tensioning mechanism may include, for example, mating spaced nuts/slits/teeth that are configured to grip the tensioning belt. FIG. 26 shows an example fastening feature 855 extending from a lower surface 853 of the belt fixation device 850.

As shown in FIG. 27A, the application of tension to the tensioning belts 830, 832, 834 and 836 causes the incision to open due to forces applied by the flexible edge protection structure. The geometry and positioning of the flexible edge protection structure, and the location of the fixation devices, can be selected in order to facilitate the opening of the incision opening according to a desired shape. For example, as shown in FIGS. 27A-27C, the geometry of the flexible edge protection structure (or the geometry of sub-modules of the flexible edge protection structure) may be selected to facilitate the opening of the incision such that a cylindrical component 860, such as an endoscope or trocar, can be inserted into the incision.

The incorporation of this embodiment in the process of endoscope incision can provide several advantages. Firstly, the soft texture and smooth coating of the edge protection members can act as a protection layer between endoscope and incision edges so that accidental tearing can be prevented. Secondly, the tensioning belt ensures even opening of the incision with precisely controlled tension. The tensioning of the incision is controlled under the skin plastic deformation limit. Thus, preventing visible scar formation for the incision, achieving “scar-free” endoscopic incision.

While the tension may be applied manually to the tensioning belts, in other example implementations, a motor may be employed to control an amount of tension applied to the tensioning belt, with optionally integrated force/tension sensors being employed to provide feedback signals for controlling the applied tension.

In other example implementations in which the retraction mechanism is autonomously actuated (e.g. by a robotic arm or by a motor integrated with the retraction mechanism that is interfaced with the control and processing circuitry), the maximum width of the incision may be defined and limited by the control and processing circuitry that controls the retraction mechanism. The maximum width (e.g. a width that avoids inelastic deformation at the incision ends/vertices) may be prescribed, for example, by a relationship dependent on the incision length. This relationship may be determined, for example, via experimental investigations involving the characterization of local tissue strain for different incision widths and lengths, and/or, for example, via modeling (e.g. via a trained machine learning algorithm).

This relationship may be employed, for example, to determine a suitable incision length needed to facilitate the opening of the incision to a selected incision width (e.g. an incision width that is sufficient for the passage of a given surgical tool). In some example implementations, this may be performed intraoperatively to determine a suitable increase in incision length that is needed to support the widening of the incision to a selected increased width without causing inelastic tissue deformation and associated scar tissue formation. In example implementations in which the laser pulses are delivered by a robotic assembly, the robotic assembly may be controlled, by the control and processing circuitry, to elongate the incision by the computed amount in order to facilitate the requisite widening of the incision.

Indeed, surgical incisions are commonly widened during operations, for example, to stretch the incision to accommodate the insertion of one or more surgical tools, or, for example, to facilitate visual access of a region of interest within the body. The present inventors have found that when conventional incision methods are employed, the opening angles of the incisions are usually uncontrolled such that the opening angle exceeds the elastic limit of the skin, causing plastic deformation to deeper skin layers at the incision corners, thus once again causes such cascade described above which ultimately leads to scaring. In other words, the unattended opening of incision causes damage or tears the incision corners.

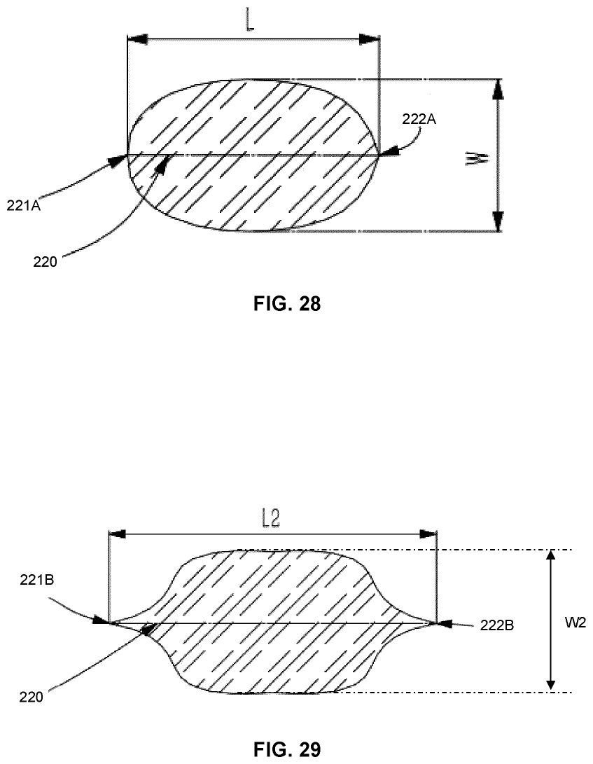

FIG. 28 illustrates such a case, in which an originally planned incision guideline 220A on tissue surface 10 has a length of L, and a maximal opening width of W. When the incision is made at the exact length of incision guideline 220 and is opened with maximum opening width W, shear stress will be concentrated at the two ends of the incision 221A and 222A. Once the shear stress exceeds the ultimate shear stress limit due to the unintentional increase of the maximal opening width W, plastic (inelastic) deformation will occur at incision edges 221A and 222A, leading to scar tissue formation. However, as shown in FIG. 29, if a relationship is known between the incision length and the maximum incision width that avoids inelastic deformation, this relationship can be employed to determine a suitable increase in incision length, from L to L2, that accommodates an increase in the incision width from W to W2 while avoiding inelastic deformation of the tissue at the incision edges 221B and 222B.

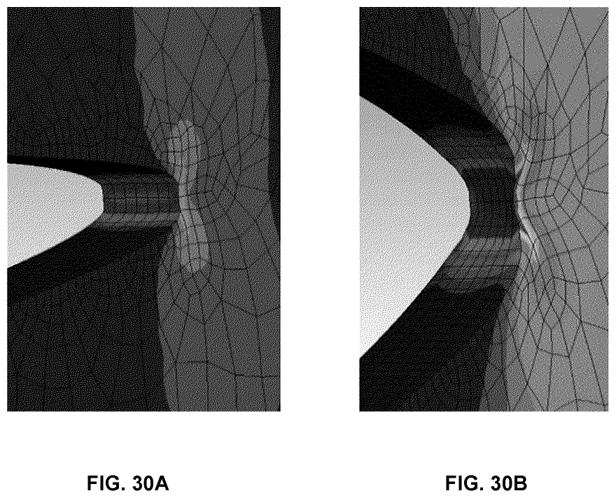

FIGS. 30A and 30B present results from finite element method (FEM) analysis illustrating the effect of incision widening on the spatial stress distribution within the incision corner regions. The incision skin model was created to mimic the condition of performing incisions such as underarm, thyroid, axillary, McBurney incision. The choice of incision types depends on the type of surgery where aesthetic outcomes matter to the patient. This model is also representative to other types of surgical incisions such as minimal invasive surgery incision and midline incision because of the similarities in incision shape. The incision model is created with a length of 3.5 inches, an initial width of 300 μm, and a depth of 5 mm. The length of the incision is determined as the average incision length of above-mentioned surgical incision types. The initial width is estimated by benchtop experiments conducted with cutting skin using pIRL. The depth is determined to be 1-2 mm larger than the average thickness of skin epidermis layer (which is 1-4 mm), thereby ensuring that the incision is cutting through the epidermis and reaches the dermis (fatty lipids) layer. Example incision parameters can be obtained, for example, according to the following references: (i) Ní Annaidh A, Bruyère K, Destrade M, Gilchrist M D, Otténio M. Characterization of the anisotropic mechanical properties of excised human skin. J Mech Behav Biomed Mater. 2012 January; 5(1):139-48. doi: 10.1016/j.jmbbm.2011.08.016. Epub 2011 Aug. 31. PMID: 22100088; (ii) Liang, Xing, and Stephen A. Boppart. “Biomechanical Properties of In Vivo Human Skin From Dynamic Optical Coherence Elastography.” IEEE Transactions on Biomedical Engineering 57, no. 4 (Apr. 9, 2010): 953-59. doi:10.1109/TBME.2009.2033464; (iii) Kalra, Anubha & Lowe, Andrew. (2016). An Overview of Factors Affecting the Skins Youngs Modulus. Journal of Aging Science. 4. 10.4172/2329-8847.1000156; and (iv) Pawlaczyk M, Lelonkiewicz M, Wieczorowski M. Age-dependent biomechanical properties of the skin. Postepy Dermatol Alergol. 2013; 30(5):302-306. doi:10.5114/pdia.2013.38359

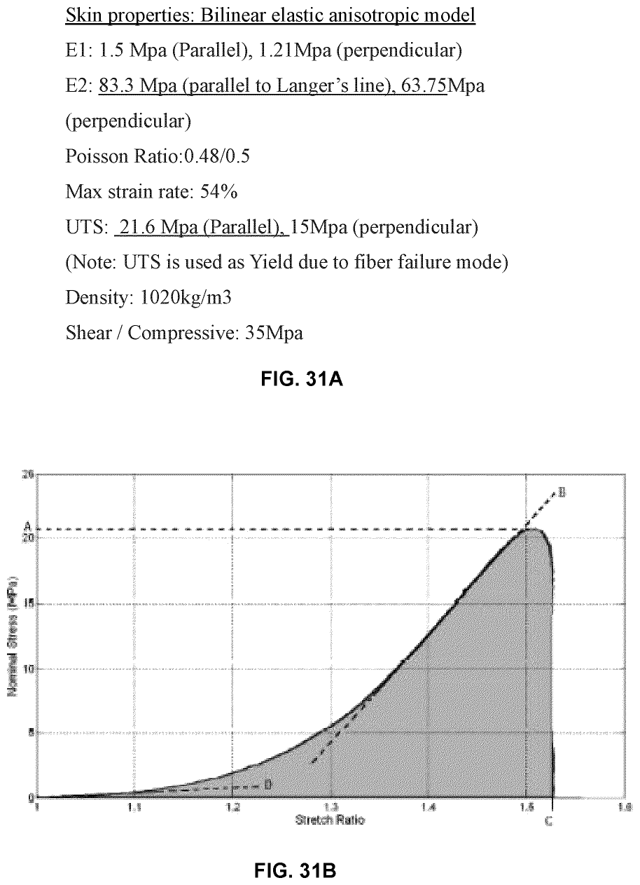

The tissue elastic parameters are shown in FIG. 31A and a bilinear elastic anisotropic model was used in this simulation. Because all surgical incisions are planned parallel to Langer's line, the incision corner will experience tension in the direction perpendicular to Langer's line during the opening of incision. Thus, the yield stress perpendicular to Langer's line and 15 MPa is used as the inelastic deformation threshold. To better represent the skin model, data extrapolated from Ní Annaidh et al. (Ní Annaidh A, Bruyère K, Destrade M, Gilchrist M D, Ottenio M. Characterization of the anisotropic mechanical properties of excised human skin. J Mech Behav Biomed Mater. 2012 January; 5(1):139-48. doi: 10.1016/j.jmbbm.2011.08.016. Epub 2011 Aug. 31. PMID: 22100088; FIG. 31B) is added to the elastic parameters used in the FEM simulation.

As can be seen in the figures, high stress regions are located at the corners of the incision. As the incision opening increases, the edge stress increases to a point at which the elastic limit is exceeded, causing inelastic (plastic) deformation of the incision edges (shown as a darker region), leading to the stimulation of fibroblast formation, and associated scar tissue formation.

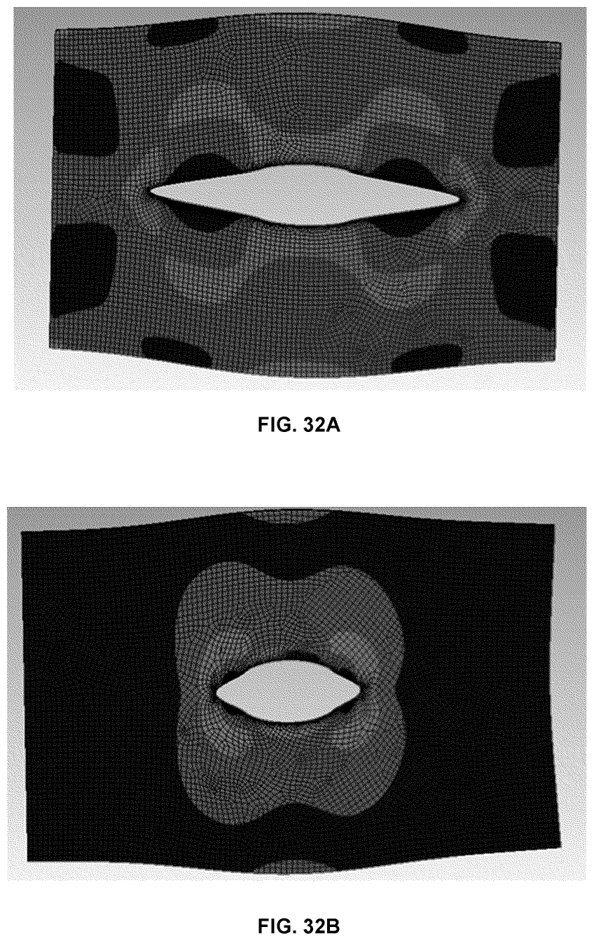

FIGS. 32A and 32B show two simulation models with different incision length of 61 mm and 129 mm and the same incision opening width. As can be seen in the figure, the incision corner stress experienced by the incision with the longer incision length is smaller than that experienced by the incision with the shorter incision length.

FIG. 33A plots the simulated dependence of incision corner stress on incision opening width for an example incision length of 99 mm. As can be seen from the graph, the maximum incision width that results in elastic deformation of tissue in the incision corners is calculated to be 86 mm.

FIG. 33B plots the dependence of incision corner stress on incision width for several different incision lengths, demonstrating how the maximum incision width for elastic deformation of corner tissue increases with incision length. This plot enables the determination of a relationship between maximum incision width and incision length, which is shown in FIG. 33C. As can be seen in the figure, the dependence of maximum incision width on incision length was fitted to a linear relationship. In some example implementations, this relationship may incorporate a safety margin, for example, to accommodate variations in tensile properties of skin, such as variations that can occur among different anatomical regions, ages, or other demographic variables. Examples of safety margins include 5%, 10%, 15%, 20% and 25%. Although the relationship in FIG. 33C is referred to as providing a maximum width as a function of incision length for avoiding inelastic tissue deformation at the incision corners, the relationship may be inverted to provide the minimum incision length for as a function of incision width to avoid inelastic tissue deformation at the incision corners.

In some example embodiments of the present disclosure, an incision closure structure is provided that facilitates alignment (registration) and contact of the incision edges after the surgical procedure is completed for healing in the absence of sutures. The incision closure structure secures the edges of the incision and holds the cut tissues edges together, in close contact, with sufficient spatial registration to avoid visible scarring.

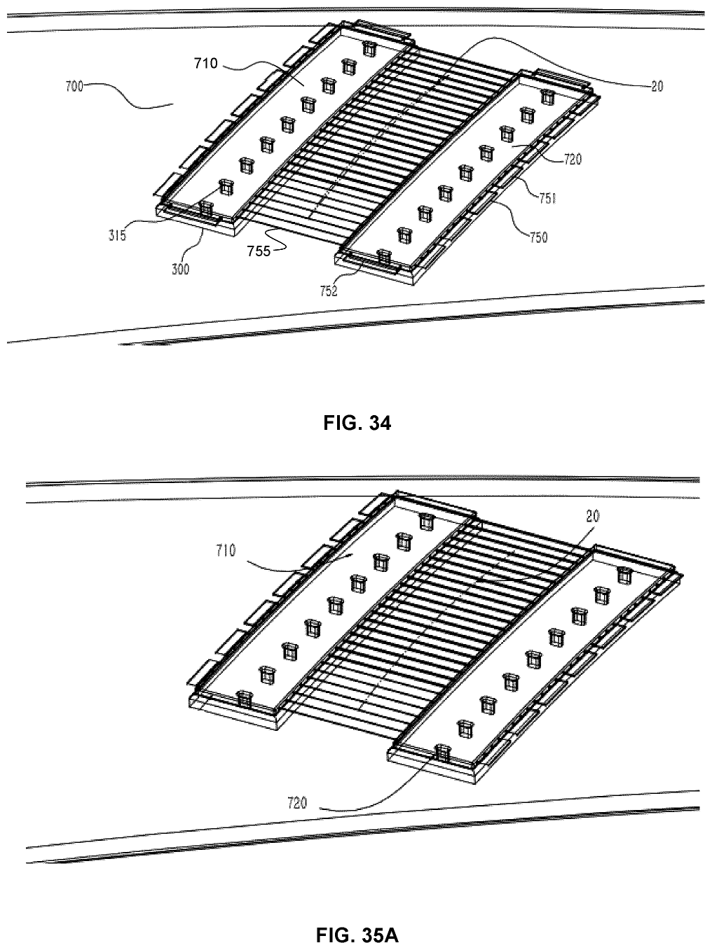

An example implementation of an incision closure structure is shown in FIGS. 34 and 35. The example incision closure structure 700 includes first and second lateral members 710, 720 that are positioned on opposing sides of the incision 20, such that the incision is resides between the first and second lateral members 710, 720. The first and second lateral members 710, 720 are removably secured, either directly or indirectly, to the skin surface 10.

As shown in the figures, the incision closure structure 600 includes a tensioning mechanism 750 that can be actuated to reduce the separation between the first and second lateral members 710, 720, thereby closing the incision and bringing the adjacent incision edges into registration and contact. The tensioning mechanism 750 is secured to the first and second lateral members 710, 720. Non-limiting examples of tensioning mechanisms include ziplines, pulling tabs, threaded mechanisms, and suction cups.

One or more components of the incision closure structure 700 may be shaped to conform to the curvature of the skin surface and may be flexible (e.g. formed from a flexible material or a flexible mechanical structure).

The tissue closure structure 700 may be initially secured relative to the skin surface in an initial configuration shown in FIG. 34. As a consequence of the attachment (either direct or indirect) of the lateral members 710, 720 to the skin surface, actuation of the tensioning mechanism 750 to decrease the separation between the first and second lateral members 710, 720 results in the closing of the incision, as shown in FIG. 35.

In one example tension mechanism, after the adhesion of lateral member 710 and 720, the tensioning mechanism 750 can be operated manually. Pull tabs 751 jointed with tensioning strings 755 can be operated to bring both incision edges close to each other. The tensioning strings 755 may be connected to the pull tabs 751 via an attachment mechanism. For example, the tensioning strings 755 can be sandwiched between two rigid pull tabs 751. In another example implementation, the puling tabs 751 can be fused with the ends of tensioning strings 755 using glue, heat shrink, melt-joint, or other adhesives. In some example implementations, pull tabs may be provided only one side of the device. However, FIGS. 34 and 35 illustrate an example implementation in which pull tabs 751 are provides on both sides to center the compression force on the incision line. Since the skin surface is not rigid, when pulling only from one side, the underlying lateral member (310 or 320) will move along the force direction, which can cause uneven compression along the incision line. Moreover, the pulling force may be balanced by skin under the two lateral members, while if only one side is pulled, the necessary force needed to close incision may be applied all on one side of the device. As shown in FIG. 34, additional pull tabs 752 may be provided in for aligning the incision edges in a second (e.g. orthogonal) direction, thereby preventing incision edge misalignment in the direction parallel to the incision.

As shown in FIG. 35B and FIG. 35C (which shows a detail region 725 of FIG. 35B), pull tabs 751 and 752 may be secured with griping teeth 730 that will act as an attachment mechanism. The tensioning strings 755 are threaded through the lateral members 710, 720 and can move freely in the through holes (not shown in the figure). The end of the through hole of each lateral member is equipped gripping teeth 730. As shown in FIG. 35C, as the tensioning string 755 is pulled through the lateral member 720, the gripping teeth 730 can grip the pull tab 751, preventing it from moving back to previous position. The gripping teeth mechanism illustrated in the figure shows one non-limiting illustration of an example location of the gripping mechanism. Alternative examples of suitable attachment mechanisms include, but are not limited to, a zip tie knot, self-gripping teeth, and spring-loaded clips. Upon tensioning the pulling strings separately, incision edges can be brought to close registration, e.g., in three degrees of freedom.

The figures illustrate an example implementation in which the support substrate 300 is initially secured to the skin surface of the subject and the first and second lateral members 710, 720 are indirectly secured to the skin surface via attachment to the support substrate 300 (e.g. via engagement features 315 on the support substrate 300 and corresponding engagement features (not shown) on the first and second lateral members 710, 720, or via an adhesive). This illustrated embodiment is beneficial in that the same support substrate 300 may be employed both during the formation of the incision (e.g. via support of a guide structure), opening and protection of the incision during a surgical procedure, and subsequently to support the incision closure structure 700 during for incision edge registration and healing after the surgical procedure. Alternatively, the first and second lateral members 710, 720 may be removably adhered directly to the skin surface via a suitable adhesive as described above.

In some example implementations, the present sutureless tissue closure device is capable of achieving a registration of adjacent incision edges with a tolerance of less than 100 microns, with differences not visibly noticeable. The alignment of incision edges within this tolerance has been found to promote healing and avoid the presence of visible scar tissue, provided that the incision has been formed, opened, and closed, according to one of the preceding example embodiments that avoid the inelastic deformation of tissue and thus prevent tissue necrosis and scar tissue formation. After some healing time in which the tensile strength of the incision reaches a particular value, the remaining fixtures are removed.

The example embodiments described above may be employed for a wide variety of surgical procedures that involve the formation of incisions in skin, such as, but not limited to, appendix removal, breast augmentation, eye lid reshaping, thyroid procedures with incision across the throat, and other known surgical interventions that require incisions.

Embodiments of the present disclosure may also be employed in applications involving scar tissue removal. In such applications, an incision guide (e.g. as described above) may be employed to demarcate a specific region of the tissue to be removed. Pristine tissue may be harvested from another region, or, for example, grown from the patients' own stem cells, and put in place or the skin is drawn to together with edges protected and held in place to create contact, fibroblast formation and healing without visible scar tissue formation. This procedure allows scar-free removal of tissue that is both psychologically impairing but often times limits functions.

The above example embodiments for facilitating scar-free surgery can also be applied to internal surgeries on organs, all soft tissue, nerve decompression procedure where the occurrence of scar tissue limits the efficacy of the surgery. For example, scar tissue is particularly known to be a problem in spinal surgery and unnatural connection of scar tissue leads to future aggravation of the nerve and pain response. In some example implementations, a scar-free surgical procedure employing the preceding example embodiments may utilize a pre-defined reference plane, for example, by securing a structure of the appropriate shape to define the location of the incision. As described above, moderate tension may be beneficial to allow advancement of the laser surgical cutting tool within the linear elastic response of the corresponding tissue. To maintain substantially scar-free tissue after forming the incision, the closing of the incision may be performed such that the incision edges are positioned for closure under linear elastic response. Moreover, bonding of the tissue edges with registration and/or alignment, and optionally bonding with a means of holding the tissue secured during healing can be beneficial in closing the wound and reestablishing the tissue matrix with minimal fibroblast formation.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1.-61. (canceled)

62. A system for forming an incision, said system comprising:
a laser system configured to generate laser pulses;
a scanning system configured to direct the laser pulses onto a skin surface of a subject and to scan the laser pulses relative to the skin surface for forming the incision;
a tensioning mechanism configured to apply tension across the incision during formation of the incision;
said laser system being configured to generate the laser pulses with a wavelength such that absorption of the laser pulses by skin tissue is predominantly due to excitation of vibrational modes of water within the skin tissue;
said laser system and said scanning system being configured to respectively generate and deliver the laser pulses such that:
a pulse duration is shorter than a first time duration required for thermal diffusion out of a laser irradiated volume of skin tissue and shorter than a second time duration required for a thermally driven expansion of the laser irradiated volume of skin tissue;
the pulse duration and a pulse fluence result in a peak pulse intensity below a threshold for ionization-driven ablation to occur within the laser irradiated volume of skin tissue; and
the pulse fluence is sufficiently high such that the laser irradiated volume of skin tissue is ablated and such that any residual energy is insufficient to induce substantial laser-induced scar tissue formation;
wherein said tensioning mechanism is configured to apply to sufficient tension to maintain a line-of-sight between an output aperture of said scanning system and a distal region of the incision during formation of the incision while preventing substantial tension-induced scar tissue formation.

63.-64. (canceled)
